(19) United States
(12) Patent Application Publication
(10) Pub. No.: US 2015/0045237 A1
(43) Pub. Date: Feb. 12, 2015

(54) METHOD FOR IDENTIFICATION OF THE SEQUENCE OF POLY(A)+RNA THAT PHYSICALLY INTERACTS WITH PROTEIN

(71) Applicant: MAX-DELBBRUCK-CERTRUM FUER MOLEKULARE MEDIZIN, Berlin (DE)

(72) Inventors: Markus Landthaler, Berlin (DE); Mathias Munschauer, Berlin (DE); Alexander Baltz, Berlin (DE)

(21) Appl. No.: 14/385,501

(22) PCT Filed: Mar. 18, 2013
(86) PCT No.: PCT/EP2013/055569

§ 371 Date: Sep. 16, 2014

(30) Foreign Application Priority Data
Mar. 16, 2012 (EP) 12159836.1
Jul. 6, 2012 (EP) 12175269.5

(51) Int. Cl. C12Q 1/68; C12N 15/113

(52) U.S. Cl. CPC C12Q 1/6874; C12N 15/113; C12N 2310/11
USPC 506/4; 435/6.11; 506/16; 536/24.5

(57) ABSTRACT

The invention relates to an in vitro method for identifying the sequence of one or more poly(A)+RNA molecules that physically interacts with protein. The present invention provides a method to define the protein-bound transcriptome under any given cellular condition, such as a disease condition or after treatment with any given substance, drug, or other cellular perturbation. The invention also relates to a method for identification of a drug target and a method for the identification of one or more biomarkers, preferably for identification of a panel of biomarkers, for any given medical condition, comprising the method of the invention.

The invention relates to an in vitro method for identifying the sequence of one or more poly(A)+RNA molecules that physically interacts with protein. The present invention provides a method to define the protein-bound transcriptome regions under any given cellular condition, such as a disease condition or after treatment with any given substance, drug, or other cellular perturbation. The invention also relates to an anti-sense oligonucleotide targeted against the sequence of a poly(A)+RNA molecule identified using the method, a method for identification of a drug target and a method for the identification of one or more biomarkers, preferably for identification of a panel of biomarkers, for any given medical condition, comprising the method of the invention.

The present invention relates in a preferred embodiment to a photoreactive nucleoside-enhanced UV-crosslinking and oligo(dT) affinity purification approach to globally map the sites of protein-poly(A)+RNA interactions in mammalian cells and other animal cell culture systems. Protein occupancy profiling on poly(A)+RNA by next-generation sequencing of protein-crosslinked RNA fragments using the method of the present invention provides a transcriptome-wide view of the interaction sites of the mRNA-bound proteome and reveals widespread binding of proteins to 5′ and 3′ untranslated regions (3′UTRs) as well as coding regions of messengerRNAs (mRNAs).

The invention therefore relates to an in vitro method for identifying the sequence of one or more poly(A)+RNA molecules that physically interact with protein, comprising formation of covalently linked poly(A)+RNA-protein complexes via cross-linking, isolation of poly(A)+RNA-protein complexes by binding of poly(A)+RNA-protein complexes with oligo(dT) oligonucleotides, ribonuclease treatment and removal of unbound poly(A)+RNA, followed by removal of total protein, and identification of poly(A)+RNA sequences, preferably by cDNA library preparation and sequencing.

### BACKGROUND INFORMATION

Protein-RNA interactions are fundamental to core biological processes, such as mRNA splicing, localization, degradation and translation. During and immediately after transcription, nascent mRNAs associate with proteins to form messenger ribonucleoprotein (mRNP) complexes that mediate and regulate most aspects of mRNA metabolism and function. Throughout their life cycle, mRNP complexes consist of a dynamically changing repertoire of proteins that define the processing, cellular localization, as well as the decay and translation rate of specific mRNAs. Posttranscriptional regulation occurs at a significant level, as evidenced by recent studies that have shown that the correlation between mRNA transcript abundance and protein copy number is relatively low, ranging from 0.41 to 0.6 (Nagaraj et al., 2011; Schwanhausser et al., 2011; Vogel et al., 2010). Moreover, alternative splicing of pre-mRNAs has emerged as key regulatory mechanism accounting for the proteome diversity in metazoan organisms (Nilsen and Graveley, 2010; Wang et al., 2008).

The mammalian genome has been predicted to encode about 600 RNA-binding proteins (de Lima Morais et al, 2011), based on the presence of one or more catalytic or non-catalytic domains that can interact with RNA. However, several proteins implicated in other cellular processes exhibit RNA-binding activity despite the absence of recognizable RNA-binding domains. Among them, the cytosolic aconitase (also known as iron-regulatory protein 1; IRP1) post-transcriptionally regulates specific target mRNAs depending on cellular iron levels (Kennedy et al., 1992). This and other examples of RNA-binding activity of unexpected proteins highlight the need to systematically catalogue the cellular repertoire of RNA-binding proteins in order to define the system that regulates the posttranscriptional fate of mRNAs.

More than 30 years ago, the first attempts were made to isolate and analyze the poly(A)+RNA-bound proteome by oligo(dT) sepharose chromatography. Purifications of mRNPs from in vitro UV-irradiated polysomal fractions (Greenberg, 1979), from UV-irradiated intact cells (Wagenmakers et al., 1980) and untreated cells (Lindberg and Sundquist, 1974) revealed the association of a specific set of proteins with mRNA. Later on, similar methods were applied to characterize hnRNP particles and to identify the mRNA polyadenylate-binding protein (Adam et al., 1986; Choi and Dreyfuss, 1984). Recently, screening and oligo(dT) purification procedures were used to provide the first comprehensive catalog of yeast mRNA-binding proteins (Scherrer et al., 2010; Tsvetanova et al., 2010). However, methods for comprehensive identification of mammalian RNA-binding proteins have remained elusive.

A prerequisite for our understanding of the function of RNA-interacting proteins is a systematic identification of their binding sites and the definition of their RNA targets. Current genomic approaches use UV crosslinking and immunoprecipitation (CLIP) of mRNA-RBP complexes in combination with next generation sequencing to identify RBP binding sites (Konig et al., 2010; Licatalosi et al., 2008). One recently developed method, PAR-CLIP, employs the photoreactive thionucleosides, 4-thiouridine and 6-thioguanosine, to increase the crosslinking efficiency between protein and RNA and to provide near nucleotide resolution of the RNA-binding site (Hafner et al., 2010). This approach is however limited to particular proteins, as it relies on IP-based approaches, that pull down essentially only those RNA molecules that interact with any given particular protein of interest.

The similar methods of PAR-CLIP (US2011/0287412), CLIP (Ule et al, Science 2003, US2011/0076676) and iCLIP (US2011/0269647) have been recently described. However, none of these methods provides a combination of deep-sequencing with the binding of poly(A)+RNA-protein complexes using poly(A)+RNA-binding oligonucleotides, preferably oligo(dT) oligonucleotides. The use of the oligo(dT) oligonucleotides as a separation/purification method provides a global approach to elucidating poly(A)+RNA-protein interactions not previously thought possible. This global approach subsequently enables enormous depth in analytic accuracy, providing simultaneous and unbiased information on multiple biomarkers and drug targets for anti-sense technology that was previously thought to be impossible to obtain.

Poly(A)+RNA-isolation methods have been disclosed in the art in the context of proteomic studies that demonstrate identification of RNA-bound protein (Schmidt et al, Mol. Biol. Rep, 2010). After RNA isolation the associated proteins are subsequently eluted and separated using SDS-PAGE before MS analysis. No cross-linking is applied. Earlier disclosures of the prior art that enable RNA analysis using photoreactive thionucleosides for crosslinking protein to RNA were limited in their scope of analysis by selective isolation procedures using immunoprecipitation (see above, in addition to WO 2010/014636). Through such methods RNA-molecules were isolated that bound a specific protein, which was determined by the choice of antibody applied in the IP reaction. However, as discussed in more detail below, simple combination of methods for poly(A)+RNA-isolation and deep sequencing of isolated RNA material is not technically feasible due to high background RNA levels. This technical feasibility issue has however been solved by the inventors, who for the first time show an effective combination of poly(A)+RNA-isolation and subsequent sequencing of isolated RNA material that was that was specifically bound by proteins as indicated by TC mutation.

Application of the present invention to a human cell line identifies around 800 proteins directly interacting with mRNA. One third of these proteins, among them transcription factors, kinases, a deubiquitinating enzyme, and DNA repair proteins, were neither previously annotated nor could be functionally predicted to bind RNA. Protein occupancy profiling on mRNA reveals detailed information on which RNA sequences are bound by protein, showing for example that large stretches in 3′ UTRs are covered by the mRNA-bound proteome, with numerous binding sites in regions harboring disease-associated nucleotide polymorphisms.

### SUMMARY OF THE INVENTION

In light of the prior art the technical problem to be solved by the invention is the provision of a method for an unbiased identification of all protein-RNA interaction sites. The present invention relates in a preferred embodiment to a photoreactive nucleoside-enhanced UV-crosslinking and oligo(dT) affinity purification approach to globally map the sites of protein-mRNA interactions in mammalian cells and other animal cell culture systems. Protein occupancy profiling on poly(A)+RNA by “next-generation” sequencing of protein-crosslinked RNA fragments using the method of the present invention provides a transcriptome-wide view of the interaction sites of the mRNA-bound proteome and reveals widespread binding of proteins to coding sequences and 5′ and 3′ untranslated regions (3′UTRs) of mRNAs.

The present invention provides a method to define the protein-bound transcriptome under any given cellular condition, such as disease condition or after treatment with any given substance, drug, or other cellular perturbation.

The invention therefore relates to an in vitro method for identifying the sequence of one or more poly(A)+RNA molecules that physically interacts with protein, comprising:
a) formation of poly(A)+RNA-protein complexes via cross-linking,
b) isolation of poly(A)+RNA-protein complexes by
   binding of poly(A)+RNA-protein complexes with poly(A)+RNA-binding oligonucleotides, preferably to oligo(dT) oligonucleotides, and
   removal of unbound poly(A)+RNA, followed by
c) removal of total protein, and
d) identification of poly(A)+RNA sequences.

It was entirely surprising that a combination of deep-sequencing after isolation of poly(A)+RNA-protein complexes using poly(A)+RNA-binding oligonucleotides would lead to reliable and sensitive identification of RNA-protein interaction sites.

Although poly(A)+RNA-isolation methods are as such known in the art, the combination of isolation of poly(A)+RNA, using preferably via oligo(dT) oligonucleotides, with subsequent deep sequencing represents a technically challenging procedure. Simple combination of known methods for poly(A)+RNA-isolation and subsequent sequencing of isolated material is not technically feasible. The combination of approaches applied in the present invention required the inventors to overcome significant compatibility issues, which ultimately have led to unexpectedly positive outcomes.

Replacing the known antibody-based IP approach directly with isolation based on oligo(dT) oligonucleotides initially provided only negative results. After formation of poly(A)+RNA-protein complexes via cross-linking and subsequent isolation of poly(A)+RNA-protein complexes with poly(A)+RNA-binding oligonucleotides, analysis of the isolated RNA provided no effective read-out on protein-bound RNA sequences. As the inventors of the present invention were able to demonstrate, and subsequently overcome, the background RNA levels (comprising of significant amounts of unbound RNA) after oligo(dT)-isolation were simply too high to enable analysis of the isolated RNA.

The invention is therefore characterised by the removal of unbound poly(A)+RNA, preferably after RNA isolation and before removal of total protein. Without this additional RNA-removal step in the method of the present invention analysis of the bound RNA molecules is technically impossible due to interfering high background RNA observed by “next-generation sequencing”.

In a preferred embodiment the method of the present invention is characterised in that the cross-linking is carried out by UV irradiation of cells treated with photoreactive nucleosides, such as 4-thiouridine and/or 6-thioguanosine.

In a preferred embodiment the method of the present invention is characterised in that the cross-linking is carried out by
a) introducing a photoreactive nucleoside into living cells wherein the living cells incorporate the photoreactive nucleoside into RNA transcripts during transcription thereby producing modified RNA transcripts and
b) irradiating said cells at a wavelength significantly absorbed by the photoreactive nucleoside to covalently cross-link a binding site on the modified RNA transcripts to one or more binding proteins, whereby
c) the wavelength is preferably greater than 300 nm.

Photoreactive nucleosides, such as 4-thiouridine and/or 6-thioguanosine, provide a particularly effective method for cross-linking. The subsequent mutation induced by the incorporation of a photoreactive nucleoside that has been cross-linked to protein enables effective sequencing and comparison to sequence databases to identify protein interaction sites in a fast and efficient manner, effectively enabling “next-generation” sequencing to be applied in genome-wide analyses.

In a preferred embodiment the method of the present invention is characterised in that the isolation of poly(A)+RNA-protein complexes is carried out using oligo(dT) oligonucleotides attached to a solid support material, preferably by
a) forming a soluble extract of the cells,
b) addition of poly(A)+RNA-binding oligonucleotides, preferably oligo(dT) oligonucleotides, attached to a solid support material to said extract,
c) washing the RNA-protein complexes that are bound to said poly(A)+RNA-binding oligonucleotides, preferably oligo(dT) oligonucleotides, attached to a solid support material under denaturing conditions, and
d) treating the extract with a nuclease thereby removing unbound poly(A)+RNA.

The use of a solid support enables simple separation of bound and unbound material. Although not an essential aspect of the invention, the use of solid-support mediated isolation is compatible with high throughput analysis and enables the analysis of multiple samples in parallel without extra experimental burden.

In a preferred embodiment the method of the present invention is characterised in that unbound poly(A)+RNA is removed via
a) treatment with one or more RNA-hydrolyzing enzymes, such as RNAse, and/or benzonase, more preferably RNAse I, as it exhibits no nucleotide bias for RNA degradation, thereby providing unbiased and efficient removal of unwanted or interfering RNA,
b) precipitation of protein-poly(A)+RNA complexes, preferably by ammonium sulphate precipitation and/or other protein precipitation methods such as Et-OH, and/or
c) separation according to size, such as by gel electrophoresis, preferably by SDS-PAGE and subsequent transfer of protein-RNA complexes to nitrocellulose.

The removal of unbound poly(A)+RNA is a defining feature of the invention and is important for enabling the analysis as described herein. The removal of unbound RNA can be carried out using various methods. For example, RNA-hydrolyzing enzymes and/or precipitation methods may be applied. The most preferred method is the use of ammonium sulphate, or other effectively similar means for precipitation of protein-RNA complexes, in combination with electrophoresis and transfer of said complexes to nitrocellulose before analysis. Protein-RNA complexes are therefore enriched by ammonium sulphate precipitation and then separated by SDS-PAGE, before being blotted onto nitrocellulose. RNA can be extracted from the nitrocellulose membrane by proteinase treatment and nucleic acid purification, for example by phenol/chloroform extraction.

It was entirely surprising that ammonium sulphate precipitation and subsequent electrophoresis and nitrocellulose transfer leads to efficient isolation of RNA-protein complexes without loss of material. The reduction of RNA background was achieved whilst maintaining specificity and sensitivity.

Ammonium sulphate precipitation is preferred over other methods of concentrating proteins, as it efficiently precipitates proteins, while nucleic acids remain largely soluble. Thus protein bound RNA fragments are enriched in the precipitate and background RNA is further removed by transfer of separated protein-RNA complexes to nitrocellulose, which specifically retains proteins but not free RNA. Alternative protein precipitation methods can be applied, but the inventors observed a surprising and beneficial reduced level of background RNA when using ammonium sulphate precipitation, in comparison to other methods.

In one embodiment the method of the present invention is characterised in that total protein is removed via protease treatment, such as protease K treatment. Proteinase K is a highly processive enzyme without any amino acid sequence bias and provides a suitable method for releasing bound RNA.

In a preferred embodiment the method of the present invention is characterised in that poly(A)+RNA sequences are identified via cloning poly(A)+RNA molecules into cDNA libraries followed by sequencing of said libraries.

In one embodiment the method of the present invention is characterised in that the identification of a sequence of a poly(A)+RNA molecule that physically interacts with protein is determined by
a) identification of a mutation in the sequence of said poly(A)+RNA molecule by sequencing of the purified protein-bound poly(A)+RNA molecules and comparison of said sequence to a reference sequence,
b) whereby the mutation is preferably defined as replacement of a deoxythymidine of the reference sequence by a deoxycytidine, or replacement of a deoxyguanine of the reference sequence by a deoxyadenine in the cDNA of the protein-crosslinked purified poly(A)+RNA molecule of 4-thiouridine and 6-thioguanine labelled cells, respectively, and
c) the sequence of the binding site extends either side of the mutation for at least 1 nucleotide, preferably from 1 to 20 nucleotides.

In one embodiment the method of the present invention is characterised in that the protein-interaction site is a protein-coding transcript or non-coding transcript.

A further aspect of the invention relates to a kit for identifying a protein-interaction site on poly(A)+RNA transcripts, the kit comprising:
a) a thiouridine and/or thioguanosine analog and/or thiouridine and/or thioguanosine analog-supplemented tissue culture medium,
b) reagents for removal of unbound RNA, such as reagents for the precipitation of RNA-protein complexes,
c) reagents for oligo(dT) affinity purification, and
d) reagents for protein precipitation
e) adapters and primers for small RNA cloning.

A further aspect of the invention relates to one or more anti-sense oligonucleotides targeted against the sequence of a poly(A)+RNA molecule identified using the method of any of the preceding claims, preferably for use as a medicament, more preferably for the treatment of a medical disorder associated with physical interaction between a protein and said poly(A)+RNA sequence. Considering the method of the invention enables identification of protein-bound RNA sequences, in particular those sequences bound specifically according to disease-state or cell-type, the generation of anti-sense oligonucleotides binding potentially protein-bound RNA sequences represents one aspect of the invention. Subsequent formulation of an RNA sequence identified by the present invention into a pharmaceutical composition, preferably with a pharmaceutically relevant carrier, such as are known in the art, requires no undue or inventive effort by a skilled person and is therefore a further aspect of the present invention.

In one embodiment the oligonucleotide of the present invention is characterised in that the oligonucleotide is targeted against a sequence of a poly(A)+RNA molecule comprising a single nucleotide polymorphism (SNP) provided in FIGS. 40 and 41 and Table S7 as a medicament for the treatment of a medical disorder associated with said SNP, such as those disorders disclosed in Table S7. Table S7 discloses specific sequences which are characterised by disease-associated SNPs and are (when in RNA form) bound by RNA-binding proteins, implicating these sequences are targets for anti-sense-based targeting approaches. For example, gain of function SNPs that lead to disease could be countered by targeting said sequences with anti-sense oligos, subsequently leading to reduced expression of said SNP-containing genes and subsequently preventing development of said disease.

In one embodiment the oligonucleotide of the present invention is characterised in that the oligonucleotide binding to the poly(A)+RNA molecule results in changes in expression of the protein for which the poly(A)+RNA molecule codes, either by ribosome disruption, regulation of translation and/or RNA degradation induced by blockage of the binding site of RNA-interacting proteins using anti-sense oligonucleotides. Modulation of splicing may also be achieved by the oligonucleotide of the present invention

A further aspect of the invention relates to a method for identification of a drug target comprising the method according to any one of the preceding claims, whereby a protein-bound sequence of poly(A)+RNA molecule identified via the method of the preceding claims represents a drug target for treatment with anti-sense oligonucleotides that bind the protein interaction site on the poly(A)+RNA molecule.

A further aspect of the invention relates to a method for optimizing a therapeutic antisense oligonucleotide by using the method as described herein, whereby the sequence of said oligonucleotide is modified according to the protein-binding characteristics of the poly(A)+RNA target molecule, as identified using the method described herein. A significant number of anti-sense molecules are in clinical development and many may bind regions of an RNA template that are also bound by protein. By using the present method the specific sequence of the RNA molecule that binds protein can be determined, thereby enabling modification of the anti-sense molecule as desired, wither to bind a protein-binding site or to avoid one. The present invention therefore enables more detailed consideration of anti-sense strategies in medicine by providing an extra level of data with regard to RNA-protein interactions in addition to the sequence of the RNA molecule itself.

A further aspect of the invention relates to a method for the identification of one or more biomarkers, preferably for identification of a panel or collection of biomarkers, for any given medical condition comprising the method according to any one of the preceding claims, whereby
a) the method is carried out on samples obtained from healthy subjects and affected subjects suffering from said condition, whereby
b) protein-bound sequences of poly(A)+RNA molecules are identified as biomarkers for the medical condition when the presence, extent and/or quantity of protein-binding at the protein-bound sequence of said poly(A)+RNA molecule is significantly different between the two samples.

In one embodiment of the invention the cloning and sequencing is carried out as follows:
a) the RNA of isolated cross-linked complexes is reverse-transcribed, thereby generating cDNA transcripts with one mutation wherein the photoreactive nucleoside is transcribed to a mismatched deoxynucleoside;
b) cDNA transcripts are amplified thereby generating amplicons;
c) nucleotide sequences of the amplicons having at least 15 nucleotides are determined;
d) sequences of the amplicons are aligned against a reference sequence; and
e) sequences of the amplicons aligned against the reference sequence are analysed so as to identify the binding site, wherein the sequences of each amplicon having a mutation resulting from the introduction of the photoreactive nucleoside is considered to be a valid amplicon comprising at least a portion of a binding site on the RNA transcript and enable single nucleotide resolution of crosslinking sites.

In one embodiment of the invention the identification of the sequence further comprises determining the sequence of a consensus motif, wherein the determination comprises using the mutation as an anchor and comparing the sequence surrounding the mutation to the reference sequence, wherein the mutation is within a sequence window that includes the mutation plus at least one nucleotide on either side of the mutation.

In one embodiment the identification of the sequence is characterized in that the sequence window includes one to twenty nucleotides on either side of the mutation. One nucleotide downstream and one upstream would make a 3 nt recognition sequence. Such a sequence region could be sufficient for binding and is therefore relevant for the present invention.

In one embodiment the identification of the sequence is characterized in that the mutation is at the center of the sequence window.

In one embodiment the identification of the sequence is characterized in that the reference sequence is a genomic sequence.

In one embodiment the identification of the sequence is characterized in that the genomic sequence is a sequence that produced the RNA transcript.

In one embodiment the identification of the sequence is characterized in that the reference sequence is a synthetic RNA sequence.

In one embodiment the identification of the sequence is characterized in that the reference sequence is derived from an expressed sequence tag database.

In one embodiment the identification of the sequence further comprises identifying a feature required for interaction of the protein-interaction site.

In one embodiment the identification of the sequence is characterized in that aligning the sequences of the amplicons comprises determining which amplicons have a mutation wherein a deoxythymidine and deoxyguanine of the reference sequence is replaced by a deoxycytidine and deoxyadenine, respectively, in the amplicons.

In one embodiment the identification of the sequence is characterized in that analyzing the sequences of the amplicons comprises determining which amplicons have only one mutation wherein a deoxythymidine and deoxyguanine of the reference sequence is replaced by a deoxycytidine and deoxyadenine, respectively, in the amplicons.

In a preferred embodiment of the invention the photoreactive nucleoside is a thiouridine analog.

In a preferred embodiment of the invention the thiouridine analog is 2-thiouridine; A-thiouridine; or 2,4-di-thiouridine.

In a preferred embodiment of the invention the thiouridine analog is substituted at the 5 and/or 6 position substituents selected from the group consisting of methyl, ethyl, halo, nitro, NR¹R² and OR³ wherein R¹, R² and R³ independently represent hydrogen, methyl or ethyl.

In a preferred embodiment of the invention the photoreactive nucleoside is a thioguanosine analog.

In a preferred embodiment of the invention the thioguanosine analog is 6-thioguanosine.

A further aspect of the invention relates to an in vitro method for identifying one or more proteins that physically interact with poly(A)+RNA, comprising:
formation of poly(A)+RNA-protein complexes via cross-linking,
binding and purification of poly(A)+RNA-protein complexes using poly(A)+RNApoly(A)+RNA-binding oligonucleotides, preferably oligo(dT) oligos,
removal of total RNA, and
identification of proteins via mass spectrometry.

In a preferred embodiment the proteins are separated by gel electrophoresis and/or enzymatically digested into peptide fragments, preferably with trypsin, and subsequently analysed via mass spectrometry, whereby protein identity is derived from comparing measured peptide mass to predicted peptide mass from a database.

In a further embodiment the method is characterised in that quantitative mass spectrometry is performed using SILAC, whereby a control sample is obtained from cells grown in culture medium comprising a suitable SILAC isotope that exhibits a different mass from the isotope in the medium of the cells used to obtain the sample to be analysed.

A further aspect of the invention is therefore a poly(A)+RNA-interacting protein selected from Table S2, in particular the sub-group of Table S2 as a medicament or drug target, preferably for the treatment of a medical disorder associated with physical interaction between said protein and an poly(A)+RNA molecule.

### DETAILED DESCRIPTION OF THE INVENTION

The inventors utilise the fact that a photoreactive nucleoside undergoes a structural change upon crosslinking to protein, and is subsequently identified as a mutation in cDNA that is prepared from the modified mRNA. This effect, the sequencing of cDNA and comparison of sequences to reference sequences is disclosed in detail in WO 2010/014636, which we hereby incorporate in its entirety by reference. The mutated cDNA can be analyzed by exploiting the mutation, thereby providing a means of distinguishing UV-crosslinked target sites from background RNA fragments that were captured but not initially crosslinked to the moiety. Such an analysis dramatically increases the recovery of target sites that were crosslinked, reduces the risk of scoring false positives of target sites, and allows for extraction of sequence information of the target site.

As used herein the term “protein” that “physically interacts” or “binds” with the RNA refers to any substantially protein entity that binds to an RNA protein binding site. Examples of proteins include, but are not limited to, proteins, protein complexes, or portions or fragments thereof, including protein domains, regions, sections and the like. Proteins include one or more RNA-binding proteins (RBP), RNA-associated proteins or combinations thereof. In addition to protein, a protein complex may comprise, for example, nucleic acid components in ribonucleoprotein complexes (RNP), e.g., miRNA, piRNA, siRNA, endo-siRNA, snoRNA, snRNA, tRNA, rRNA, ncRNA, IncRNA or combinations thereof. In RNP complexes, RNA guides and participates in target RNA binding. Protein complexes may also include RNA helicases, e.g. MOV10, and Proteins containing nuclease motifs, e.g. SND1.

As used herein, the term “protein binding site” or “interaction site” refers to that portion, region, position or location of an RNA transcript in which at least one interaction with a protein occurs. Such interaction may include at least one direct interaction between a nucleotide of the RNA transcript and an amino acid of the protein. A binding site or sites of an RNA transcript may be found at a structured or unstructured region of the RNA transcript. It is also contemplated that more than one binding site may exist for any one RNA transcript. Further, binding sites of RNA transcripts may involve non-contiguous nucleotides of the RNA transcript. Such binding sites are contemplated when structure, such as, for example, a stem loop, is involved in binding.

A “photoreactive nucleoside” refers to a modified nucleoside that contains a photochromophore and is capable of photocrosslinking with a protein. Preferably, the photoreactive group will absorb light in a spectrum of the wavelength that is not absorbed by the protein or the non-modified portions of the RNA.

As referred to herein, the “living cell or cells” may be part of a cell culture, a cell extract, cell line, whole tissue, a whole organ, tissue extract, or tissue sample, such as, for example, a biopsy or progenitor cells as from bone marrow or stem cells. The living cell can be from a healthy source or from a diseased source, such as, for example, a tumor, a tumor cell, a cell mass, diseased tissue, tumor cell extract, a pre-cancerous lesion, polyp, or cyst or taken from fluids of such sources. The cells can be any kind of cells, for example, cells from bacteria and yeast, animals, especially mammalian cells, and plants.

Once RNA transcripts have been produced, or at a time at which transcription should have produced transcripts within the living cell or cells, the living cell or cells comprising the modified RNA transcripts are then irradiated. The irradiation is at a wavelength which is significantly absorbed by the photoreactive nucleoside such that covalent cross-links are formed between the modified RNA transcript and a protein and the RNA is not damaged. The minimum wavelength can be 300 nm, preferably 320 nm, and more preferably 340 nm. The maximum wavelength can be 410 nm, preferably 390 nm, and more preferably 380 nm. Any combination of minimum and maximum wavelength values can be used to describe a suitable range. The optimal wavelength is approximately 330 nm for a thiouridine analog. The optimal wavelength for a thioguanosine analog is approximately 310 nm.

Irradiation forms covalent cross-links between the modified RNA transcript and a protein spatially located close enough to said modified RNA transcript to undergo cross-linking The Part or parts of a modified RNA transcript which are close enough contact to have undergone cross-linking with a protein can be considered binding sites. Thus, binding sites are covalently cross-linked to binding proteins. (For example, see FIG. 1.)

Covalent cross-linking allows the use, in some embodiments of the present invention, of rigorous purification schemes, such as, for example, oligo(dT) oligonucleotide purification and separating complexes an SDS-PAGE. In some embodiments, the covalent bond enables partial cleavage of RNA molecules without affecting their protein binding by the use of nucleases.

The modified RNA transcripts, or portions thereof, which are not covalently cross-linked upon irradiation to one or more binding proteins are removed. The resulting constructs are termed “cross-linked segments” or “RNA-protein complexes” These “cross-linked segments” or complexes include the portion of the modified transcript that comprises the binding site as well as at least the portion of the protein that was subject to cross linking. The binding site therefore contains at least one photoreactive nucleoside through which the binding site is cross-linked to the protein. The complexes also may include additional nucleotides of the modified RNA transcript that are not bound to the binding moiety.

The cross-linked segments are then isolated. The preferred isolation method relates to isolation of poly(A)+RNA-protein complexes using oligo(dT) oligonucleotides attached to a solid support material, preferably by forming a soluble extract of the cells, addition of poly(A)+RNA-binding antisense oligonucleotides attached to a solid support material to said extract, washing the RNA-protein complexes that are bound to said poly(A)+RNA-binding antisense oligonucleotides attached to a solid support material, and treating the extract with a nuclease thereby removing unbound poly(A)+RNA.

A “poly(A)+RNA molecule” is to be understood as any RNA molecule that comprises a polyA-sequence attached to it. The poly(A) sequence is commonly known as a tail that consists of multiple adenosine monophosphates; in other words, it is a stretch of RNA that has adenine bases. In eukaryotes, polyadenylation is part of the process that produces mature messenger RNA (mRNA) for translation.

Preferably, magnetic beads, such as Dynabeads, are used as the substrate. The beads can be easily collected by a magnet. Preferably, precipitate, i.e., the isolated “cross-linked segments,” are washed.

RNA-protein complexes are treated with a ribonuclease nuclease. The nuclease trims the regions of the modified transcripts that are not cross-linked to binding proteins. It is contemplated, in one embodiment, that the nuclease would remove, or trim, the entire portion of a modified transcript that is not cross-linked to a binding moiety. However, since trimming can occur in various places an a modified RNA transcript which are not cross-linked to binding proteins, the population of “cross-linked segments” may include “cross-linked segments” with various species of “flanking segments”.

Preferably, the nuclease is ribonuclease I (Escherichia coli). Ribonuclease I preferentially hydrolyzes single-stranded RNA to nucleoside 3′-monophosphates via nucleoside 2′,3′-cyclic monophosphate intermediates.

Protein-RNA complexes are preferably enriched by ammonium sulphate precipitation and separated by electrophoresis, preferable SDS-PAGE, and blotted onto nitrocellulose to further removed non-crosslinked RNA.

Precipitation is known in the art for enriching proteins. The present invention encompasses as precipitation any method which leads to effective precipitation of RNA-protein complexes, and therefore preferably encompasses any given protein precipitation method. Common protocols relate to acetone/TCA precipitation, chloroform methanol, ammonium sulphate or ethanol precipitation. Further examples are given below. Precipitation serves to concentrate and fractionate the target product from various contaminants. The underlying mechanism of precipitation is to alter the solvation potential of the solvent and thus lower the solubility of the solute by addition of a reagent. The solubility of proteins in aqueous buffers depends on the distribution of hydrophilic and hydrophobic amino acid residues on the protein's surface. Hydrophobic residues predominantly occur in the globular protein core, but some exist in patches on the surface. Proteins that have high hydrophobic amino acid content on the surface have low solubility in an aqueous solvent. Charged and polar surface residues interact with ionic groups in the solvent and increase solubility. Knowledge of amino acid composition of a protein will aid in determining an ideal precipitation solvent and method. Salting out is the most common method used to precipitate a target protein. Addition of a neutral salt, such as ammonium sulphate, compresses the solvation layer and increases protein-protein interactions. As the salt concentration of a solution is increased, the charges on the surface of the protein interact with the salt, not the water, and the protein falls out of solution (precipitates). As a result, less water partakes in the solvation layer around the protein, which exposes hydrophobic patches on the protein surface. Proteins may then exhibit hydrophobic interactions, aggregate and precipitate from solution. Isoelectric point precipitation is also possible. The isoelectric point (pI) is the pH of a solution at which the net primary charge of a protein becomes zero. At a solution pH that is above the pI the surface of the protein is predominantly negatively charged and therefore like-charged molecules will exhibit repulsive forces. Likewise, at a solution pH that is below the pI, the surface of the protein is predominantly positively charged and repulsion between proteins occurs. However, at the pI the negative and positive charges cancel, repulsive electrostatic forces are reduced and the attraction forces predominate. The attraction forces will cause aggregation and precipitation. The pI of most proteins is in the pH range of 4-6. Mineral acids, such as hydrochloric and sulfuric acid are used as precipitants. Addition of miscible solvents such as ethanol or methanol to a solution may cause proteins in the solution to precipitate. The solvation layer around the protein will decrease as the organic solvent progressively displaces water from the protein surface and binds it in hydration layers around the organic solvent molecules.

In a preferred embodiment, the binding proteins are removed from the “isolated cross-linked segments” to generate “isolated segments.” The protein components of the binding proteins are removed by digesting the binding proteins with a protease. Preferably, digestion is effected by Proteinase K or a homologous enzyme. Proteinase K is capable of efficiently digesting protein binding proteins, liberating RNA and yielding RNA products.

Other examples of classes of proteases or their homologues include: Aspartyl proteases, caspases, thiol proteases, Insulinase family proteases, zinc binding proteases, Cytosol Aminopeptidase family proteases, Zinc carboxypeptidases Neutral Zinc Metallopeptidases, extracellular matrix metalloproteinases, matrixins, Prolyl oligopeptidases, Aminopeptidases, Proline Dipeptidases, Methionine aminopeptidases, Serine Carboxypeptidases, Cathepsins, Subtilases, Proteasome A-type Proteases, Proteosome B-type Proteases, Trypsin Family Serine Proteases, Subtilase Family Serine Proteases, Peptidases, and Ubiquitin carboxyl-terminal hydrolases.

The “isolated cross-linked segments” and/or the “isolated segments” are then reverse transcribed to generate cDNA transcripts. Note that although it is preferred to remove the binding moiety before reverse transcription (i.e., to reverse transcribe the isolated segments), it is also possible to reverse transcribe the isolated cross-linked segments (i.e., the segments to which a whole or partial binding moiety is attached). The introduction of the photoreactive nucleoside yields a mutation in the cDNA transcript when the isolated crosslinked segment is reverse transcribed. For example, the thiouridine analog is reverse transcribed to a deoxyguanosine instead of the deoxyadenosine that is normally incorporated into the reverse transcribed cDNA by Watson-Crick base pairing. The thioguanosine analog is reverse transcribed to a deoxythymidine instead of the deoxycytidine normally incorporated by Watson-Crick base-pairing. Therefore, the mutation within the cDNA transcript is located within a binding site.

The cDNA transcripts are then amplified, thereby generating cDNA amplicons. When the thiouridine analog is reverse transcribed to produce the mutation of a deoxyguanosine instead of the deoxyadenosine, as described above, the respective cDNA transcripts, when amplified, will include a mutation wherein the expected deoxythymidine is replaced with a deoxycytidine in the amplicons.

When the thioguanosine analog is reverse transcribed to produce the mutation of a deoxythymidine instead of the deoxycytidine, as described above, the respective cDNA transcripts, when amplified, will include a mutation wherein the expected deoxyguanosine is replaced by a deoxyadenosine in the amplicons.

The reverse transcription and amplification can be performed by methods known in the art. For example, the reverse transcription to generate cDNA transcripts and amplification can be achieved using linker ligation and RT-PCR thereby generating amplified cDNA transcripts.

In one embodiment, to prepare cDNA from the “isolated cross-linked segments” and/or the “isolated segments” (i.e., the isolated small RNAs), first synthetic oligonucleotide adapters of known sequence are ligated to the 3′ and 5′ ends of the small RNA Pool using T4 RNA ligases. The adapters introduce primer-binding sites for reverse transcription and PCR amplification. Along with the “isolated cross-linked segments” and/or the “isolated segments,” the small RNA Pool typically comprises contaminants resulting from the nuclease digests of very abundant transcripts and non-coding RNAs such as ribosomal RNAs. If desired, non-palindromic restriction sites present within the adapter/primer sequences can be used for generation of concatamers to increase the read length for conventional sequencing or longer size range 454 sequencing.

As will be appreciated by those in the art, the attachment, or joining, of the adapter sequence to the “isolated cross-linked segments” and/or the “isolated segments” can be done in a variety of ways. For example, the adapter sequence can be attached either at the 3′ or 5′ ends, or in an internal position of “isolated cross-linked segments” and/or the “isolated segments.”

In one embodiment, precautions can be taken to prevent circularization of 5′ phosphate/3′ hydroxyl small RNAs during adapter ligation. For example, chemically pre-adenylated 3′ adapter deoxyoligonucleotides, which are blocked at their 3′ ends to avoid their circularization, can be used. The use of pre-adenylated adapters eliminates the need for ATP during ligation, and thus minimizes the Problem of adenylation of the Pool RNA 5′ phosphate that leads to circularization. Additionally, a truncated form of T4 RNA ligase 2, Rn12(1-249), or an improved mutant, Rn12(1-249)K227Q, can be used to minimize adenylate transfer from the 3′ adapter 5′ phosphate to the 5′ phosphate of the small RNA Pool and subsequent Pool RNA circularization. See also International Patent Application No. PCT/US2008/001227, published as WO 2008/094599, which is incorporated herein by reference in its entirety.

The length of the adapter sequences will vary. In a preferred embodiment, adapter sequences range from about 6 to about 500 nucleotides in length, preferably from about 8 to about 100, and most preferably from about 10 to about 25 nucleotides in length. The cDNA amplicons are then sequenced. The sequencing can be performed by any known means. In a preferred embodiment, the sequencing method will generate sequences of amplicons of at least about 20 nucleotides in length.

For example, the amplicons can be sequenced using “Illumina” massive parallel sequencing platform or other similar sequencing methods which yields 30 million sequences of 32, 36, 72 or 100 nucleotides in length per library and sequencing reaction. Solexa/Illumina sequencing can also be carried out conveniently at a smaller scale processing a larger sample number, i.e. yielding about 1.5-150 million reads per sample. The larger sets are obtained, if a full sequencing plate is used. (See M. Hafner, P. Landgraf, J. Ludwig, A. Rice, T. Ojo, C. Lin, D. Holoch, C. Lim, T. Tuschl, Identification of microRNAs and other small regulatory RNAs using cDNA library sequencing, Methods, 2008, 44:3-12.) Alternatively, the amplicons can be sequenced using pyrosequencing (454 sequencing, Roche), which provides up to 400,000 sequences of up to 250 nt in length for a single read. Data management and sequence analysis from small RNA cDNA libraries is best carried out in collaboration with an experienced computational biology laboratory.

The amplicons are then assessed in order to identify those that include the portion of the RNA transcript that binds to the binding moiety in vivo.

In one embodiment, first unique sequences (i.e., nonredundant sequences) are identified and counted. Preferably, by various steps, the amplicons are filtered to remove irrelevant sequences (i.e., irrelevant amplicons). For example, the amplicon sequences can be filtered in accordance with any or all for the following rules: The selected amplicons should have sufficient length to enable identification by means of sequencing or hybridization. The selected amplicons should not have highly repetitive portion(s) within their sequence.

The selected amplicons should avoid sequences that may interfere with the manipulation of RNA and DNA while performing the invention (e.g. they should not have recognition sites for restriction endonucleases used during the manipulation process). For example, the amplicons are narrowed to those more likely to include the portion of the RNA transcript that binds to the binding moiety in vivo. For example, in one embodiment, amplicons which are shorter than a certain number are removed, for example, less than 20 nucleotides or less than 15 nucleotides. Additionally, amplicons that do not map to a portion of the reference sequence being studied and/or amplicons that do not map to a portion of a known RNA sequence can be removed. Further, amplicons which contain highly repetitive portion(s) within their sequence (e.g., many multiples of TATA or GCGC) can be removed. Such sequences are referred to as “low entropic sequences”.

A “reference sequence” refers to any known sequence with which to compare an amplicon sequence. The reference sequence may be derived from a genomic sequence, a transcriptome sequence, an expressed sequence tags (EST) database, a sequence from which the RNA transcript was extracted, a known sequence library, a synthetic nucleotide sequence, a randomized RNA sequence, or a known RNA sequence. Typically, the human genomic sequence is being studied.

Next, the amplicons with overlapping sequences are “clustered.” “Clustering” refers to grouping together and aligning overlapping sequences.

In one embodiment, the quantities of amplicons in a particular cluster are then counted. For example, overlapping amplicon sequences, which differ by length simply because of a different point of digestion by a nuclease, can be counted as a cluster

In another embodiment, aligning sequences occurs without narrowing down the amplicons in quantity before analyzing the amplicons.

The greater the quantity of amplicons in a particular cluster, the more likely that those amplicons include an RNA sequence expressed in vivo as opposed to being merely noise. (For example, see FIG. 2.) (See P. Berninger, D. Gaidatzis, E. van Nimwegen, M. Zavolan, Computational analysis of small RNA cloning data, Methods, 2008, 44, 13-21.)

Noise is the low frequency amplicon counts that are due to random degradation or RNA turnover products present as background in cross-linked RNA recovered from IP or gels. In one embodiment, noise is detected by the absence of a deoxythymidine to deoxycytidine mutation when using a thiouridine analog, such as 4-thiouridine, as the photoreactive nucleoside or by the absence of a deoxyguanosine to deoxyadenosine mutation when using a thioguanosine analog, e.g., 6-thioguanosine, as the photoreactive nucleoside. Noise can also be detected by the absence of very sharp “peaks” at any given transcript. Noise is seen as a random distribution of amplicons along a transcript without characteristic mutations.

In a further embodiment, aligning the sequences of the amplicons includes determining which amplicons have a mutation (preferably, a mismatch mutation) when compared to the reference sequence. For example, aligning the sequences of the amplicons may include determining which amplicons have a mutation wherein a deoxythymidine of the reference sequence is replaced by a deoxycytidine in the amplicons, when a thiouridine analog, such as 4-thiouridine, is used as the photoreactive nucleoside.

As another example, aligning the sequences of the amplicons may include determining which amplicons have a mutation wherein a deoxyguanosine of the reference sequence is replaced by a deoxyadenosine in the amplicons when using a thioguanosine analog, e.g., 6-thioguanosine, as photoreactive nucleoside. In one embodiment, such amplicons that are determined to have a mismatch mutation when compared to the reference sequence are considered “valid amplicons.”

In a preferred embodiment, the aligning the sequences of the amplicons includes determining which amplicons have at least one mismatch mutation when compared to the reference sequence. In another preferred embodiment, the step of aligning the sequences of the amplicons includes determining which amplicons have only one mismatch mutation when compared to the reference sequence.

A “mismatch” as used herein refers to a nucleic acid base that is any other nucleic acid base located on an amplicon at a specific position compared to the nucleic acid base that is aligned to the reference sequence. For example, at Position 1 on the amplicon is a thymidine, and on the reference sequence that is aligned, at Position 1, the mismatch can be Adenosine, Guanosine, or Cytosine. The mismatch between the amplicon and reference sequence may be due to deletions, insertions, substitutions, or frameshift mutations in the amplicon or reference sequence. The sequences of the amplicons are then analyzed to determine the specific location on an RNA transcript that a given binding moiety binds in vivo, i.e., to determine the binding site. In this method, the amplicons are further narrowed down to find “valid amplicons.” A “valid amplicon” as used herein refers to an amplicon that is not noise, as described above. A “valid amplicon” includes those having a mutation resulting from the introduction of the photoreactive nucleoside. For example, one method by which to find “valid amplicons” is to use the deoxythymidine to deoxycytidine mutation. Clustered amplicons with only a single mutation with respect to the “reference sequence,” i.e., the deoxythymidine to deoxycytidine mutation, are located. It is considered that the mutation occurred upon reverse transcription as described above. Such amplicons are considered to be “valid.” Additionally, 4-Thiouridine crosslinks can induce T deletions with low frequency, which are still diagnostic.

Another method by which to find “valid amplicons” is to use the deoxyguanosine to deoxyadenosine mutation. Clustered amplicons with only a single mutation with respect to the “reference sequence,” i.e., the deoxyguanosine to deoxyadenosine mutation, are located. It is considered that the mutation occurred upon reverse transcription, as described above. Such amplicons are also considered to be “valid.”

Preferably, these “valid amplicons” are assessed in view of the total number of sequences that aligned to the region at issue, i.e., the total amplicons in a particular cluster. The total number of aligned sequences includes those sequences that have the mutation and those that do not have the mutation. The greater the percentage of the total aligned amplicons that show the mutation, the greater is the probability that the amplicons showing the mutation are “valid amplicons.”

When assessing the percentage, it is preferable to take into account the quantity of total aligned amplicons i.e., the total amplicons in a particular cluster. For example, a low percentage (e.g., 1% to 49%) is adequate to demonstrate a “valid amplicon” if the total quantity of aligned sequences is large (20 amplicons or more); and a high percentage (e.g., 50% to 100%) is adequate to demonstrate a “valid amplicon” if the total quantity of aligned sequences is small (19 amplicons or less. At least 10% of the sequences have to show the mutation to indicate a “valid amplicon.”

Once “valid amplicons” have been identified, they are further analyzed in view of the “reference sequence” to determine the presence of a consensus motif or sequence within a binding site. The binding site can be part of coding transcript or non-coding transcript of RNA. For example, the deoxythymidine to deoxycytidine mutation and/or the deoxyguanosine to deoxyadenosine mutation in the amplicon are used as an anchor for comparing the sequence surrounding the mutation to the “reference sequence.” Such surrounding sequence is termed “sequence window.”

In one embodiment, the “sequence window” includes the mutation plus at least one nucleotide on either side of the mutation. Preferably, the number of nucleotides on either side of the mutation ranges from about 5 to about 20 nucleotides. In another embodiment, the mutation is at the center of the sequence window.

As is known in the art, a number of different programs and algorithms may be used to identify whether an amplicon has sequence identity or similarity to a known sequence. Sequence identity and/or similarity is determined using standard techniques known in the art, including, but not limited to, the local sequence identity algorithm of Smith & Waterman, Adv. Appl. Math., 2:482 (1981), by the sequence identity alignment algorithm of Needleman & Wunsch, J. Mol. Biol., 48:443 (1970), by the search for similarity method of Pearson & Lipman, Proc. Natl. Acad. Sci. U.S.A., 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res., 12:387-395 (1984), preferably using the default settings, or by inspection. All references cited in this paragraph are incorporated by reference in their entirety.

In one embodiment, motif searches are conducted for the extracted sequences by computational means known in the art. Examples of methods used in conducting motif searches (i.e., consensus sequence searches) include CONSENSUS, multiple expectation maximization for motif elicitations (MEME) program, Gibbs sampling, PhyloGibbs sampling, Motif Discovery scan program (MDScan), or A1ignACE (Roth, F. P., Hughes, J. D., Estep, P. W. & Church, G. M. Finding DNA regulatory motifs within unaligned noncoding sequences clustered by whole-genome mRNA quantitation. Nat Biotechnol 16, 939-45 (1998)). For example, the MEME program finds conserved ungapped short motifs within a group of related, unaligned sequences (Bailey and Gribskov, 1998, J Comput Biol, 5:211-21). MDScan, for example, is used to identify sequence motifs from a set of identified genomic regions (Liu X S et al. (2002) Nat. Biotechnol., 20(8):835-9).

In another embodiment, more than one algorithm may be used to identify motifs for the extracted sequences.

In one embodiment, the analysis of the amplicon sequences can further include identifying a feature required for interaction of the binding site and the binding moiety. For example, evaluation of the consensus sequence of the binding site can reveal a structure, such as a stem loop, that may be required or involved in binding to the binding moiety.

Once the consensus motif of the binding site has been identified using the methods described above, it can be utilized for various clinical or research applications. For example, the binding site can be sequenced using patient DNA to identify mutations, deletion or insertions that may link a genetic alteration in an important, regulatory RNA segment to a disease condition. It is known that RNA binding proteins are essential regulators of proteins by binding to coding and non-coding RNAs and regulating their transcription, modification, splicing, nuclear export, transport and translation.

Consequently, understanding the binding site on the RNA and the identity of the bound RNA binding proteins provides opportunities for new therapies. For example, an RNA-binding protein known to affect the stability or translation of a gene can be utilized as a drug target for the regulation of the targets of the gene.

### FIGURES

FIG. 1. Illustration of the experimental setup to identify the mRNA-bound proteome and its occupancy profile on RNA. Transcripts were labeled with photoreactive nucleosides and proteins were crosslinked to RNA by 365 nm UV-irradiation. mRNP complexes were isolated by cell lysis and oligo(dT)-precipitation under denaturing conditions. For the identification of the mRNA bound proteome, mRNPs were eluted from the beads, nuclease-treated and analyzed by quantitative mass-spectrometry. To identify the protein binding pattern on RNA, mRNPs were RNAse I treated, followed by proteinase K digest to remove RNA-bound proteins. RNA molecules were converted into a cDNA library and next-generation sequenced.

FIG. 2. SDS-PAGE analysis of proteins crosslinked to polyadenylated RNA. HEK293 cells were grown in medium supplemented with 4SU and/or 6SG and UV-irradiated at 365 nm. Cells were lysed using denaturing conditions and protein-mRNA complexes were isolated by oligo(dT)-precipitation. Protein-RNA complexes were eluted from oligo(dT) beads, treated with RNAse I, separated on a SDS gradient gel and visualized by silver-staining.

FIG. 3. GAPDH mRNA depletion. qRT-PCR analysis of GAPDH mRNA in supernatants (SN1 to SN4) after each round of oligo(dT) bead precipitation (four in total) compared to GAPDH mRNA in extract before precipitations (input) shown as percent of input. The error bars display the calculated maximum and minimum expression levels that represent the standard error of the mean expression level with a 95% confidence interval.

FIG. 4. Western Blot analysis of FLAG/HA-tagged RNA-binding proteins QUAKING (QKI) and ARGONAUTE 2 (AGO2/EIF2C2) in input extract (I), supernatant after precipitation (S), and oligo(dT)-purified material (P) of UV-crosslinked and non-crosslinked cells.

FIG. 5. Read count distribution over different RNA types. mRNA was purified either from total TRIzol-extracted RNA by a single oligo(dT) precipitation (mRNA seq), or by four rounds of oligo(dT) precipitation from cellular extract of UV-irradiated and non-irradiated cells (4SU+6SG UV and 4SU+6SG no UV, respectively). Crosslinked proteins were removed by Proteinase K digest prior to RNA analysis by next-generation sequencing of recovered RNA. The read count distribution over different RNA classes (mRNA, rRNA and other) was inferred by multiplying the FPKM values with the respective length of the longest transcript of a given gene.

FIG. 6. Pair-wise correlation between RNA abundance expressed as log 2 FPKM of RNA described in (E). To assess the incorporation of photoreactive nucleoside into RNA, the 4SU and 6SG-containing RNA was purified from oligo(dT) precipitated RNA of non-crosslinked cells by biotinylation and streptavidin-pulldown (4SU+6SG purified RNA) and analyzed by next-generation sequencing. The diagonal is shown as yellow line for of each pairwise comparison whereas a LOESS regression line is shown in red.

FIG. 7. Summary of proteomic experiments. In two replicates the proteomic composition of oligo(dT)-precipitates was analyzed for “light” labeled crosslinked cells (experiments L1 and L2) and one experiment for “heavy” labeled crosslinked cells (H1). The overlap of identified proteins in different experiments is shown in the Venn diagram. Table indicates the number of identified and quantified proteins, as determined by SILAC ratios of proteins in each experiment.

FIG. 8. Comparison of the log 2 fold changes (LFC) of “heavy” to “light” SILAC ratios (H/L) of proteins quantified in biological replicates L1 and L2. Previously known RNA-binding proteins are indicated in green and known contaminants in red.

FIG. 9. As in (FIG. 8) Proteins quantified in L1 plotted against proteins quantified in label swap experiment H1.

FIG. 10. As in (FIG. 8) Proteins quantified in L2 plotted against proteins quantified in label swap experiment H1.

FIG. 11. Overview of identified mRNA-interacting proteins. Number of identified proteins belonging to different functional categories.

FIG. 12. Overview of identified mRNA-interacting proteins. Median relative number of protein molecules belonging to different functional categories as determined shown as box plots. Protein amounts were calculated as the sum of all peptide peak intensities divided by the number of theoretically observable tryptic peptides (Schwanhausser et al., 2011). The median is shown as horizontal line and the surrounding box defines the upper and lower quartile. The sample range is defined by the whiskers, while dots indicate potential outliers.

FIG. 13. Overview of identified mRNA-interacting proteins. Overlap of identified mRNA binders with proteins present in spliceosome and nucleolus.

FIG. 14. Overview of identified mRNA-interacting proteins. Number of identified proteins with specific RNA-binding domains (dark grey) was compared to respective number of RNA-binding domain containing proteins in expressed HEK293 proteome (light grey).

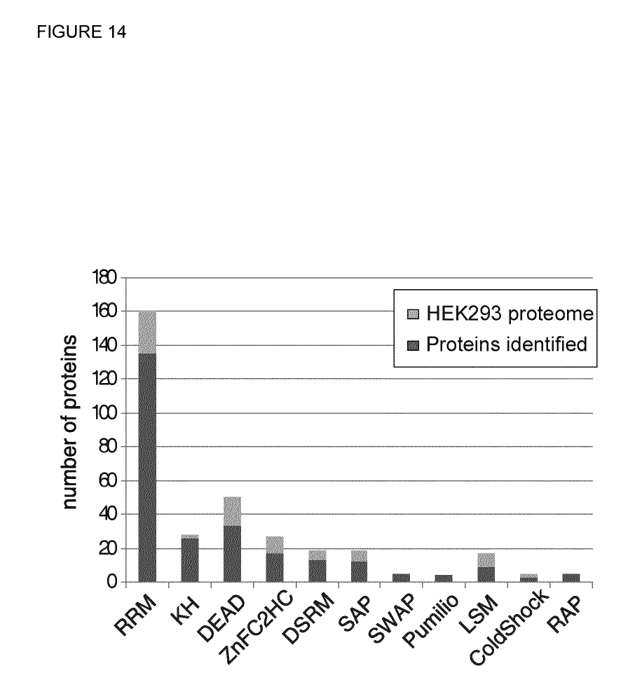

FIG. 15. Validation of RNA-binding activity of candidate mRNA binders. RNA-binding activity of candidate mRNA binders was determined by PAR-CLIP. Protein-RNA complexes were separated by SDS-PAGE and blotted onto nitrocellulose membrane. Western analysis using an anti-HA antibody confirmed the correct size and equal loading of the IPed protein. Phosphor imaging indicated efficient radioactive labeling of covalently bound nucleic acid in the mRNP complex. The assay was performed at least twice for each protein. Representative results are shown. CAPRIN1, HNRNPD, HNRNPR, HNRNPU and MYEF2 served as positive controls.

FIG. 16. As in FIG. 16. Metabolic enzymes LDHA and PGK1, both not detected in oligo(dT) precipitations, served as negative controls.

FIG. 17. As in FIG. 16. Results of PAR-CLIP assay for 21 putative mRNA binders are shown. The radioactive background signal in non-crosslinked immunoprecipitates is likely due to the presence of protein kinases.

FIG. 18. PAR-CLIP analysis of candidate RNA-binding proteins. Distribution of mRNA binding sites based on PAR-CLIP sequence clusters for the indicated proteins are shown. Absolute number and percentage distribution of sequence clusters in different transcript regions are indicated.

FIG. 19. PAR-CLIP analysis of candidate RNA-binding proteins. PAR-CLIP sequence coverage along transcript regions is shown for ALKBH5 and C22orf28.

FIG. 20. PAR-CLIP analysis of candidate RNA-binding proteins. Genome browser view of spliced and unspliced XBP1 transcript isoforms. Putative C22orf28 binding sites flanking the XBP1 intron are indicated in dark grey.

FIG. 21. Specific T-C transitions in protein occupancy profiling sequencing reads. Specific mismatches in aligned sequence reads demonstrate efficient protein-RNA crosslinking. The frequency of nucleotide mismatches in occupancy profiling reads aligned to human genome is shown for library 1. T-C mismatches are the signature of efficient crosslinking of 4SU-labeled RNA to protein.

FIG. 22. Specific T-C transitions in protein occupancy profiling sequencing reads. Specific mismatches in aligned sequence reads demonstrate efficient protein-RNA crosslinking. The frequency of nucleotide mismatches in occupancy profiling reads aligned to human genome is shown for library 2. T-C mismatches are the signature of efficient crosslinking of 4SU-labeled RNA to protein.

FIG. 23. Mapping of protein occupancy profiling sequence reads. Distribution of mapped sequence reads to different RNA types for library 1.

FIG. 24. Mapping of protein occupancy profiling sequence reads. Distribution of mapped sequence reads to different RNA types for library 2.

FIG. 25. Comparison exonic to intronic read counts in protein occupancy profiling libraries (related to FIG. 6). Comparison of exon versus intron read count for occupancy libraries 1 and 2. We defined a transcriptome-wide exon/intron sequence-normalized read count (similar to the well-known RPKM value) by calculating the number of reads mapping only to exonic or intronic regions normalized by the total number of mapped reads per million and the number of exonic or intronic nucleotides in kilobases.

FIG. 26. Correlation of protein occupancy profiling sequence coverage between two libraries. Density of transcript-wise rank correlation coefficients based on sequence coverage of two protein occupancy profiling libraries between corresponding (black solid line) and unrelated (grey dashed line) transcripts. A sliding window approach was used to compare sequence coverage over entire transcripts. Solid vertical lines indicate medians, dashed vertical lines the 5% and 95% quantiles, respectively.

FIG. 27. Correlation of protein occupancy profiling sequence coverage between two libraries. Scatterplot of median transcript-coverage values of two protein occupancy profiling libraries. The solid line represents the best linear fit. The rank correlation coefficient based on all pair-wise comparisons is indicated

FIG. 28. Reproducibility of individual T-C transitions in two protein occupancy profiling libraries. Reproducibility of individual T-C transition sites. The reproducibility was measured as the percentage of sites with a minimal number of T-C transitions, which also showed a certain number of transitions (≧1 (bold black line), ≧(dashed bold grey line), ≧(dashed thin grey line) in the replicate experiment.

FIG. 29. Correlation of position-specific number of T-C transitions in two protein-occupancy profiling libraries. Scatterplot of absolute numbers of position-specific T-C transition events for all T positions inside transcripts, which showed at least 2 transitions in one of the two replicates. The solid line indicates the best linear fit. Pearson correlation coefficient is indicated.

FIG. 30. Detailed view of occupancy profile on EEF2 gene. Browser view of genomic region encoding EEF2 gene (Human genome 18). Track A shows consensus T-C transition profile (number of T-C transitions). Track B shows consensus sequence coverage. Track C shows Phastcon conservation of placental mammals.

FIG. 31. Detailed view of occupancy profile on EEF2 3′UTR. Browser view of genomic region encoding 3′UTR of EEF2 gene (Human genome 18). Track A shows consensus T-C transition profile (number of T-C transitions). Track B shows consensus sequence coverage. Track C shows Phastcon conservation of placental mammals.

FIG. 32. Detailed view of occupancy profile in 3′UTRs (related to FIG. 11). Browser view of genomic region encoding 3′UTR of EEF2 (Human genome 18). Tracks A and B show T-C transition profiles (number of T-C transitions) for libraries 1 and 2, respectively. Tracks C and D show sequence coverage for libraries 1 and 2, respectively. Track D shows Phastcon conservation of placental mammals.

FIG. 33. Detailed view of occupancy profile on CBX3 3′UTR. Browser view of genomic region encoding 3′UTR of CBX3 gene (Human genome 18). Track A shows consensus T-C transition profile (number of T-C transitions). Track B shows consensus sequence coverage. Track C shows Phastcon conservation of placental mammals.

FIG. 34. Detailed view of occupancy profile on TP53 3′UTR. Browser view of genomic region encoding 3′UTR of TP53 gene (Human genome 18). Track A shows consensus T-C transition profile (number of T-C transitions). Track B shows consensus sequence coverage. Track C shows Phastcon conservation of placental mammals. Track D shows binding sites of individual RNA binding proteins. Black boxes indicate experimentally verified binding sites of RNA binding proteins HuR and RBM38. White boxes indicate binding sites of HuR identified by PAR-CLIP.

FIG. 35. T-C transition probability around microRNA target sites. Probability of observing T-C transitions around miRNA binding sites in protein occupancy profiling data. microRNA target sites are indicated by bold black line.

FIG. 36. T-C transition probability around microRNA target sites. Probability of observing T-C transitions around miRNA binding sites in AGO PAR-CLIP data. microRNA target sites are indicated by bold black line.

FIG. 37. T-C transition density on different transcript regions. Relative density of T-C transitions along different transcript regions, observed in protein occupancy profiles. Thin black line indicates entire transcript, thick black line indicates 5′UTR, dashed grey lines indicates CDS, thick grey line indicates 3′UTR.

FIG. 38. Number of crosslinking sites observed in 3′UTRs compared to number of available thymidines. Number of 3′UTR uridine positions with indicated number of consensus T-C transitions.

FIG. 39. Conservation of crosslinked thymidines in protein occupancy profiles. Comparison of PhyloP score of 3mer sequences centered on crosslinked T (dashed grey line) to random non-crosslinked 3mers (black line) is shown. The p-value indicates the significance of the difference of the PhyloP score distribution between crosslinked and control regions as given by a two-sample Kolmogorov-Smirnov test.

FIG. 40. Detailed view of occupancy profile around trait/disease-associated SNP rs9299. Browser view of genomic region encoding 3′UTR of HOXB5 (Human genome 18). Track A shows consensus T-C transition profile (number of T-C transitions). Track B shows consensus sequence coverage. rs9299 (black box below track B) represents a single nucleotide polymorphism located in the 3′UTR of HOXB5 that is associated with childhood obesity. Track C shows Phastcon conservation of placental mammals.

FIG. 41. Detailed view of occupancy profile around trait/disease-associated SNP rs8321. Browser view of genomic region encoding 3′UTR of ZNRD1 (Human genome 18). Track A shows consensus T-C transition profile (number of T-C transitions). Track B shows consensus sequence coverage. rs8321 (black box below track B) represents a single nucleotide polymorphism located in the 3′UTR of ZNRD1 that is associated with AIDS progression. Track C shows Phastcon conservation of placental mammals.

FIG. 42. Detailed view of protein occupancy on ACTB 3′UTR in HEK293 and MCF7 cells. Browser view of genomic region encoding 3′UTR of ACTB (Human genome 18). Tracks A and B show T-C transition profiles in HEK293 and MCF7 cells, respectively. Tracks C and D show sequence coverage in HEK293 and MCF7 cells. Track E shows Phastcon conservation of placental mammals. Bottom panel shows zoom into a 50 nt region within the 3′UTR of ACTB. Tracks F and G show T-C transition profiles for HEK293 and MCF7 in zoom in region.

FIG. 43. Detailed view of protein occupancy on ACTB 3′UTR in HEK293 and MCF7 cells. Browser view of genomic region encoding 3′UTR of ACTB (Human genome 18). Tracks A and B show T-C transition profiles in HEK293 and MCF7 cells, respectively. Tracks C and D show sequence coverage in HEK293 and MCF7 cells. Track E shows Phastcon conservation of placental mammals. Bottom panel shows zoom into a 20 nt region within the 3′UTR of ACTB. Tracks F and G show T-C transition profiles for HEK293 and MCF7 in zoom in region.

FIG. 44. Detailed view of protein occupancy on Smg7 3′UTR in undifferentiated and differentiated mouse embryonic stem (ES) cells. Browser view of genomic region encoding 3′UTR of Smg7 (Mus musculus genome 9). Tracks A and B show T-C transition profiles in undifferentiated and differentiated mouse ES cells, respectively. Tracks C and D show sequence coverage in undifferentiated and differentiated mouse ES cells, respectively. Track E shows Phastcon conservation of placental mammals. Bottom panel shows zoom into a 100 nt region within the 3′UTR of Smg7. Tracks F and G show T-C transition profiles in undifferentiated and differentiated mouse ES cells, respectively.

### EXPERIMENTAL EXAMPLES

### Optimization of mRNP Oligo(dT) Affinity Purification

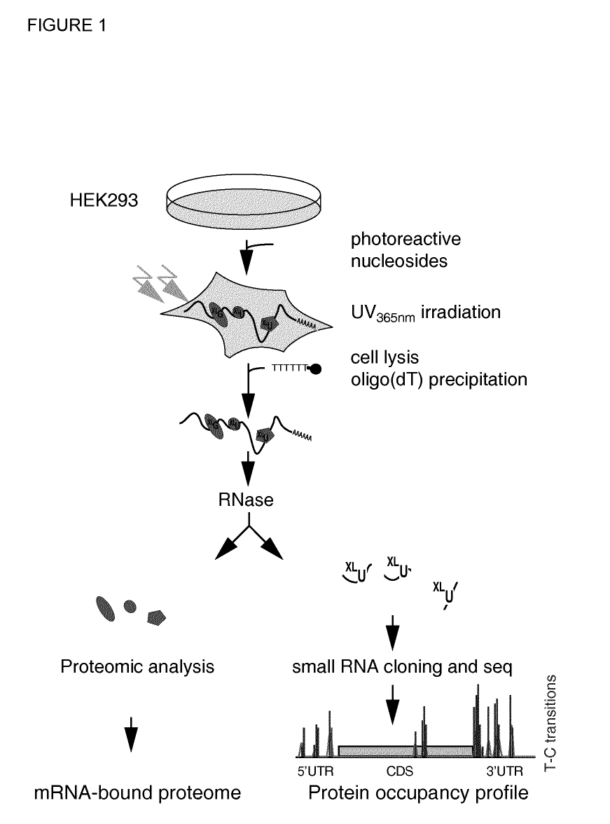

To characterize the protein-mRNA interactome, we sought to improve existing methods to identify the protein content of oligo(dT) affinity-purified mRNA ribonucleoprotein (mRNP) complexes and to determine the mRNA regions contacted by the mRNA-bound proteome (FIG. 1). A key feature of our approach is the use of photoreactive nucleoside analogs to metabolically label cellular RNA. Both 4-thiouridine (4SU) and 6-thioguanosine (6SG) are readily taken up by cultured mammalian cells and dramatically enhance the crosslinking efficiency of proteins to RNA by UV 365 nm irradiation compared to protein-RNA crosslinking at 254 nm (Ascano et al., 2011; Hafner et al., 2010). Photo-crosslinking of living cells stabilizes mRNP complexes and facilitates their isolation by oligo(dT) affinity purification (Setyono and Greenberg, 1981; Wagenmakers et al., 1980). Protein-denaturing conditions during the purification ensure a stringent isolation of proteins in direct contact with mRNA through covalent bonds and thus enable the identification of the mRNA-interacting proteins by mass spectrometry (FIG. 1). Moreover 4SU-labeled RNA, crosslinked to proteins, can readily be identified by characteristic T to C transitions in cDNA (Hafner et al., 2010) providing a way to globally identify the RNA binding sites of the mRNA-bound proteome (FIG. 1).

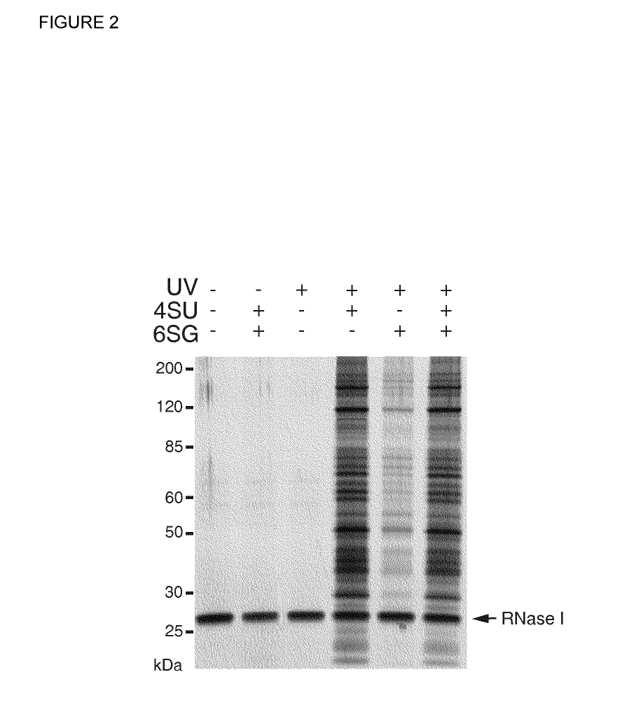

We initially tested this approach by purifying protein-mRNA complexes using magnetic oligo(dT) beads from UV-irradiated and non-irradiated intact human embryonic kidney (HEK) 293 cells after growth in medium supplemented with or without 4SU and 6SG. Resolving the RNase-treated eluate on a SDS-PAGE revealed that the combination of metabolic labeling of RNA with photoreactive nucleosides and irradiation at UV 365 nm allowed a high recovery of proteins (FIG. 2). We further examined the amount of mRNA obtained in precipitates from extracts of crosslinked and non-crosslinked cells. A qRT-PCR analysis showed that comparable amounts of GAPDH mRNA were precipitated, suggesting that labeling of RNA and UV irradiation had only a minor effect on the mRNA pulldown efficacy.

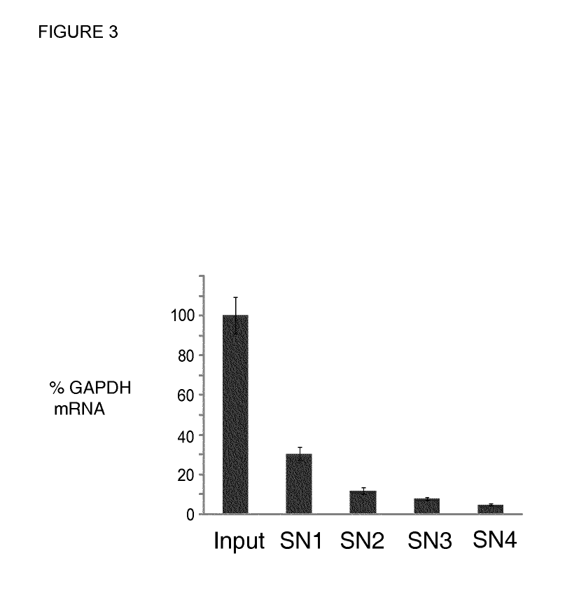

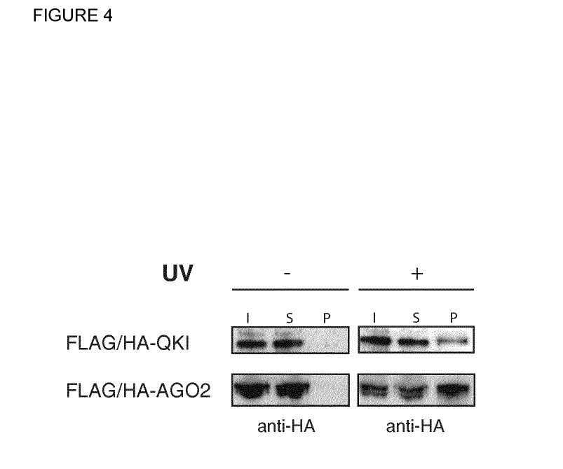

As expected when probing the oligo(dT) precipitate for the presence of known RNA-binding proteins by Western analysis, we were able to detect the heterogeneous nuclear ribonucleoprotein K (HNRNPK). However, the Argonaute protein, AGO2/EIF2C2, was not detectable after a single oligo(dT) pull down, likely due the insufficient precipitation of mRNAs and/or incomplete capture of mRNAs with shortened poly(A) tails, like microRNA/AGO targeted mRNAs. Thus, we measured the degree of depletion of GAPDH mRNA after one oligo(dT) precipitation. The GAPDH transcript is abundant and targeted by AGO proteins (Hafner et al., 2010; Kishore et al., 2011). FIG. 3 shows that only about 70% of this transcript was depleted in the supernatant when compared to input RNA. Three additional consecutive pull downs from the same extract reduced the amount of GAPDH mRNA in the supernatant to about 5% (FIG. 3). A Western analysis of the pooled eluates of four oligo(dT) purifications validated the presence of AGO2 protein (FIG. 4) as well as the RNA-binding protein QUAKING (QKI), indicating that a single or multiple consecutive oligo(dT) purifications are required to precipitate crosslinked AGO protein.

Early attempts at analysis of isolated RNA without removing unbound RNA demonstrated that a simple combination of oligo(dT)-based isolation with RNA sequencing produced poor results. Due to significant unbound RNA background, the analysis detected none or extremely low levels and therefore unusable of the mutated sequences indicative of protein-RNA cross-linking. Further development of the method, by including RNA removal, such as via enzymatic digestion and/or precipitation followed by SDS-PAGE and transfer to nitrocellulose membranes, enabled a significant increase in sensitivity of the RNA sequences bound by protein.

### Characterization of the Oligo(dT)-Purified RNA

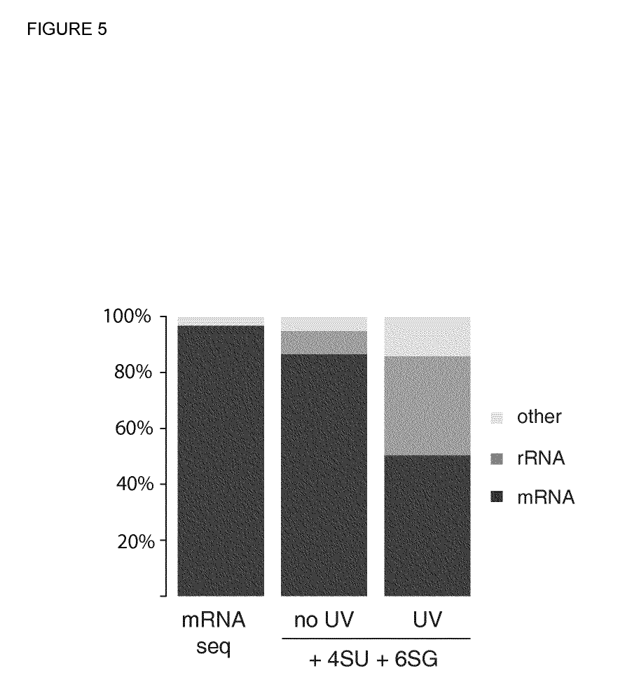

To obtain a more detailed picture of the RNA present in the pooled precipitates of four consecutive oligo(dT) pull downs, we constructed a cDNA library by random priming 4SU- and 6SG-labeled RNA derived from irradiated and non-irradiated cells. Digital gene expression analysis of the cDNA library of non-irradiated cells, labeled with 4SU and 6SG, revealed that about 88% of the sequence reads mapped to mRNA and 8% rRNA genes, whereas in RNA precipitates obtained from UV-irradiated cells the rRNA content increased to 36%, likely reflecting crosslinking of ribosomes to mRNA transcripts (FIG. 5). In contrast a standard mRNA purification procedure, involving a single oligo(dT) precipitation, of untreated cells resulted in 96% mRNA and 2% rRNA (FIG. 5).

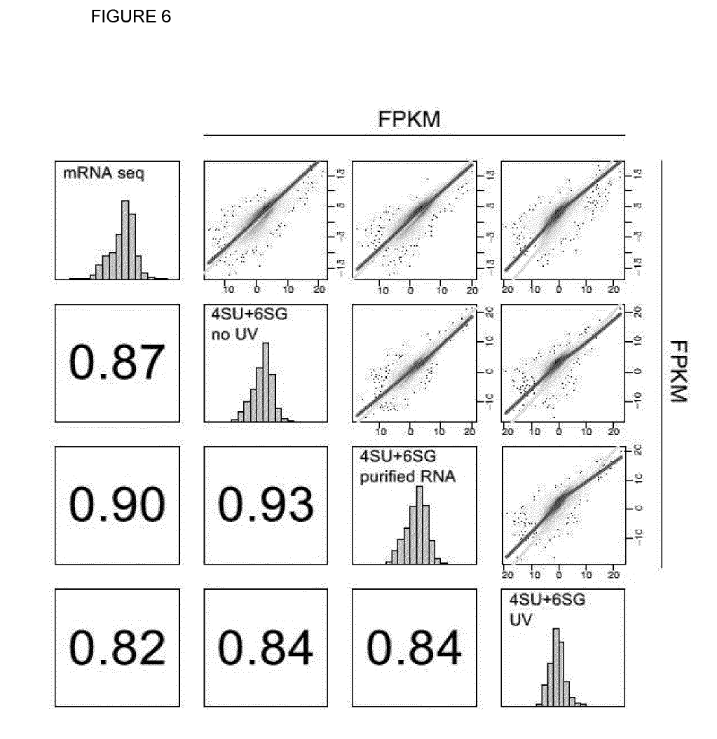

Furthermore a comparison of the different RNA libraries showed that the abundance of mRNAs obtained by a single oligo(dT) purification from untreated cells and metabolically-labeled transcripts derived from non-crosslinked and UV-crosslinked cells correlated well (Pearson correlation coefficient of 0.87 and 0.82, respectively, FIG. 6), indicating the oligo(dT)-precipitated mRNA closely reflected the cellular mRNA pool. To monitor the incorporation of photoreactive nucleotides into mRNA, we isolated 4SU- and 6SG-labeled RNA from the oligo(dT) precipitate of non-crosslinked cells. The abundance of the thionucleotide-containing RNA was in good agreement with cellular mRNA (Pearson correlation coefficient of 0.90), suggesting efficient and unbiased metabolic labeling of transcripts (FIG. 6). In summary, we concluded that 4 consecutive oligo(dT) pull downs are preferred to efficiently purify cellular mRNA-protein complexes.

Identification of mRNA-Bound Proteins by Quantitative Mass Spectrometry

To identify proteins crosslinked to mRNAs, we performed oligo(dT) purifications, as described above, and precipitates were analyzed by SILAC-based quantitative mass spectrometry (Ong et al., 2002). For this purpose, cells were grown in medium supplemented with “light” or “heavy” stable isotope labeled amino acids to compare the protein abundance in oligo(dT) precipitates of crosslinked cells to that of non-crosslinked cells. We performed two independent experiments (L1 and L2) in which the “light” labeled cells were UV-irradiated and proteins in the oligo(dT) pull down were compared to the precipitate of non-crosslinked “heavy” labeled cells. In a single “label swap” experiment (H1) the “heavy” labeled cells were crosslinked and the recovered proteins were compared to those of “light” labeled non-crosslinked cells.

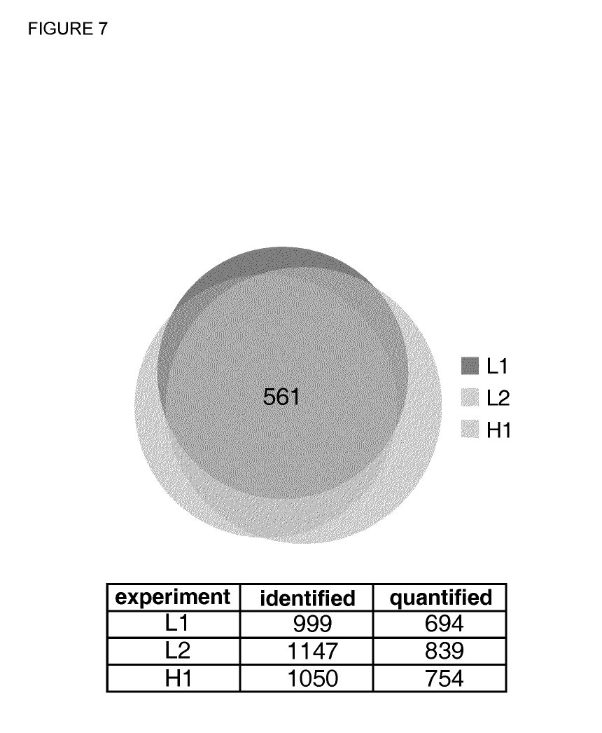

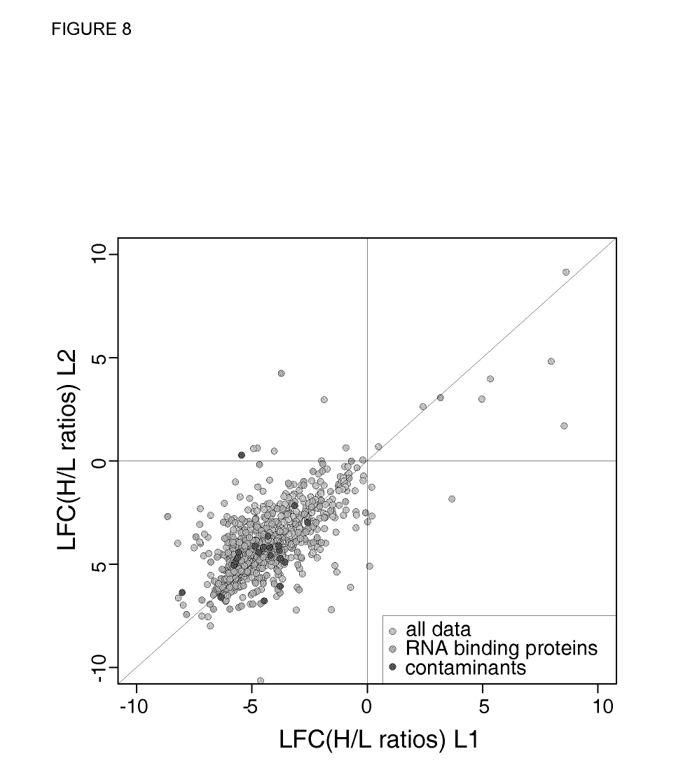

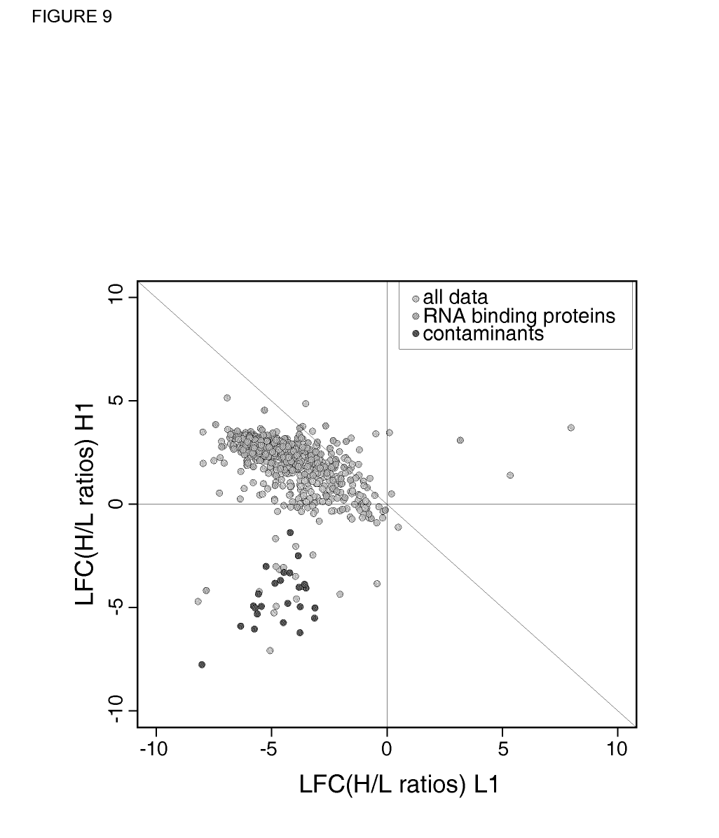

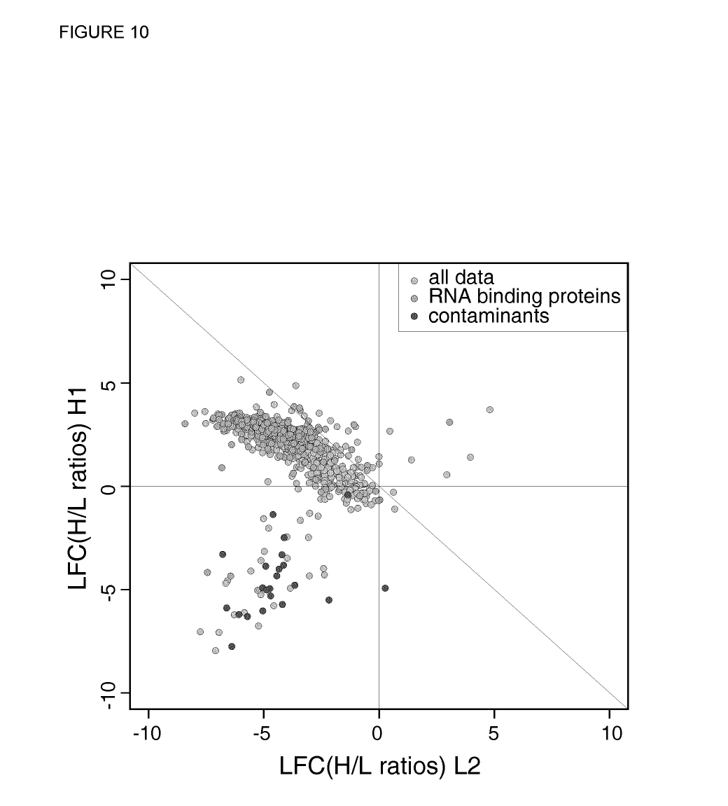

In total, we identified 1326 proteins and observed a significant overlap between experiments. 790 proteins were identified in all of the three proteomic analyses and 562 of those were quantified with at least three observed SILAC-peptide ratios in each experiment (FIG. 7). To further examine the reproducibility we compared log 2 SILAC ratios from biological replicates L1 and L2 (FIG. 8). 801 out of 827 proteins identified in both experiments were specifically enriched in the precipitates of UV crosslinked cells relative to the non-irradiated control cells (SILAC log 2 fold changes <0 in both cases). Hence, 97% of all identified proteins showed specific enrichment. In addition we observed no correlation between the fold enrichment and the cellular protein abundance (FIG. S2B), suggesting that the degree of enrichment was independent of the number of protein molecules present in the cell. Next, we plotted the log 2 SILAC ratios from both biological replicates against the label swap experiment. As expected, most SILAC ratios were inverted by the label swap (FIGS. 9 and 10). The proteins with low SILAC ratios in both the biological replicates and the label swap experiment were assumed to be contaminants such as trypsin, LysC and keratins. We therefore excluded 176 proteins with negative log 2 SILAC ratios in the label swap experiment. Among the excluded proteins were 6 RBPs: the small nuclear ribonucleoprotein polypeptide E (SNRPE), the U3 RNA-binding protein PDCD11, ELAVL3, RBM16, PA2G4, and RBPMS. In addition we applied a restrictive cut-off, requiring an enrichment of at least 3-fold in at least one of three analyses, which reduced the non-redundant list of 838 proteins to 801 (Table S2).

We further subdivided the 801 proteins into three groups. Group 1 included 505 proteins (63%), which were enriched more than 3-fold in all three proteomic analyses. 191 proteins (24%) showed an enrichment in two experiments (group 2) and 107 proteins (13%) showed enrichment in only one experiment (group 3).

Overview of Identified mRNA-Interacting Proteins

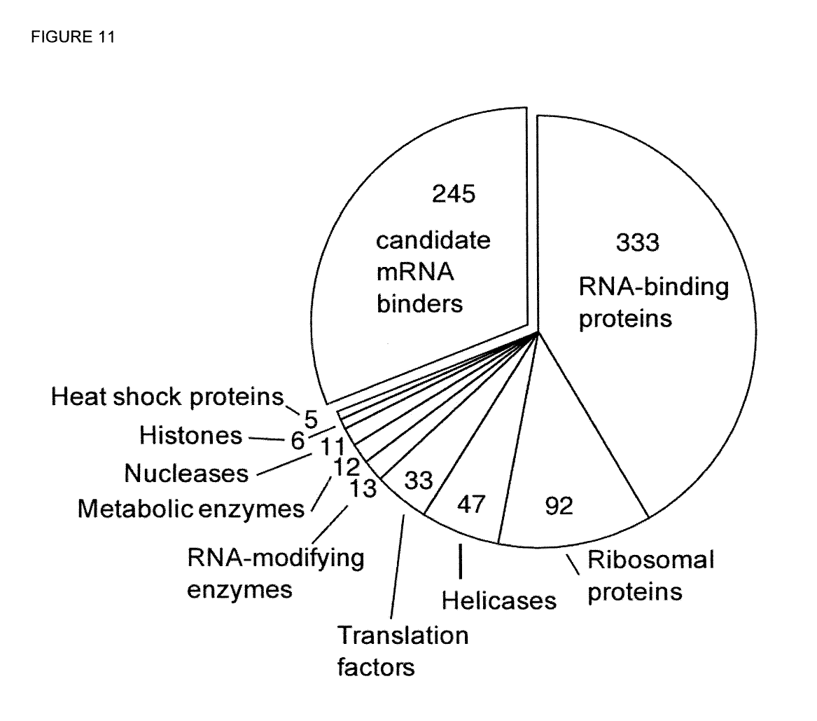

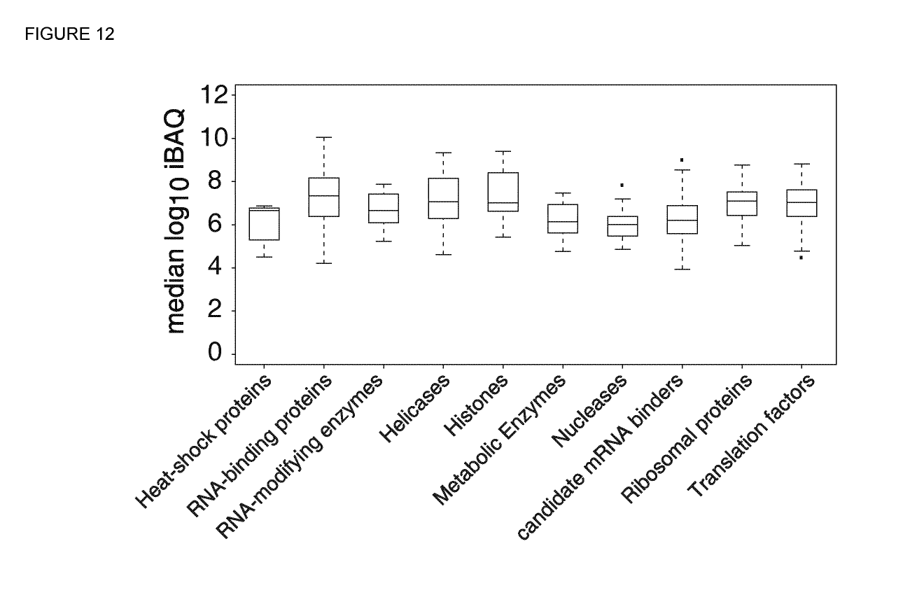

We first classified the 801 mRNA-interacting proteins into functional categories based on gene annotation. As expected, ribosomal proteins, RNA helicases, translation factors and RNA-binding proteins were most abundant, making up close to 70% of the identified proteins (FIG. 11). The low numbers of highly expressed cellular proteins such as metabolic enzymes, histones and heat-shock proteins, suggested that the oligo(dT) purification was specific. The mean relative abundance of identified proteins belonging to different functional groups was comparable (FIG. 12), indicating that the protein identification is not unduly biased towards a specific category.

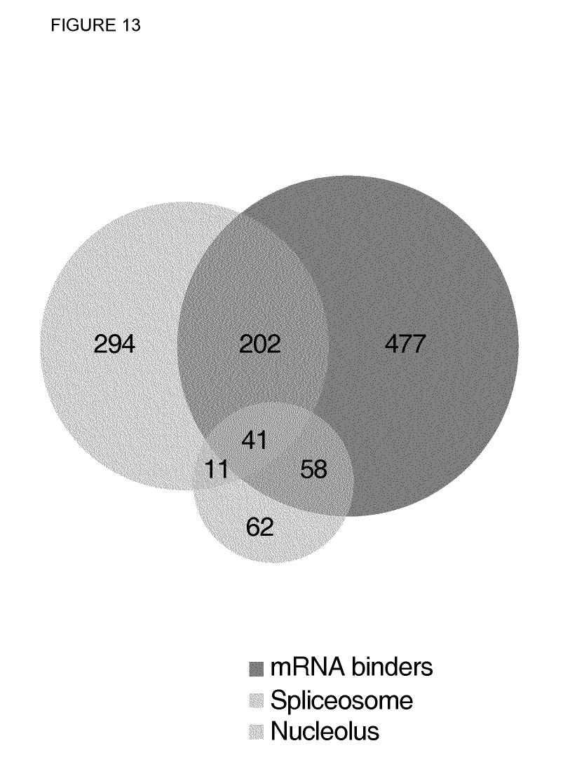

Confirming the method, we discovered RNA-interacting proteins present in complexes that influence surveillance and translation of spliced mRNAs. We detected all proteins, RBM8A/Y14, MAGOH, EIF4A3, and CASC3/BTZ, making up the core of the exon junction complex (EJC), as well as the EJC-associated proteins PNN, ACIN1, RNPS1, SRRM1, DDX39B, UPF3B and ALY/REF (Le Hir and Andersen, 2008). Additionally, we identified EIF4A1, EIF4B, EIF4E, EIF4G1, and EIF4H, all of which are present in the translation initiation complex (Jackson et al., 2010). Furthermore, the complete set of 21 HNRNP proteins, which have diverse functions in mRNA processing and transport, were discovered in this analysis. On the other hand, the identified mRNA binders only partially overlapped with sets of proteins found in nuclear RNA-containing structures. 99 out of 172 proteins detected in spliceosomal B and C complexes (Bessonov et al., 2008), were observed to interact with mRNA (FIG. 13). 243 identified mRNA interactors were also found in the nucleolus proteome (Andersen et al., 2005) (FIG. 13).

In addition to the expected mRNA-interacting proteins, we identified 267 proteins (Table S2), which have not been previously annotated as RNA-binding (FIG. 3A, others). 80% of these proteins were detected in at least 2 out of 3 proteomic analyses and about 50% were observed in all three pull-downs (Table S2). We applied an adaptation of a multiple association network integration algorithm (Mostafavi et al., 2008) to predict proteins with RNA-binding function, using gene ontology data, Interpro and Pfam domain data, gene coexpression, protein-protein interaction, and structural similarity data (Drew et al., 2011). This algorithm demonstrated strong predictive power, as evidenced by the precision-recall values for RNA-binding (see supplemental table XN.1) and by previous field-wide tests of function prediction algorithms (Pena-Castillo et al., 2008). A full description of the algorithm and benchmarking results appear in the supplemental.

After applying the algorithm to the 267 non-annotated mRNA-interacting proteins detected by our assay, 136 (54 from group 1) proteins could not be predicted as RNA binders (even at a very low precision level of >20%, and when using the function prediction algorithm in a manner that minimises false negative predictions at the expense of false positive predictions). This strongly suggests that our experiments uncovered new types of RNA-interacting proteins (RNA-binders that use new or highly divergent RNA-binding domains that occupy novel regions of the known protein association networks, Table S2). Some of our discoveries include proteins that are functionally annotated as transcription factors (JUN, NXF1), protein kinases (FASTKD1, FASTKD2, FASTKD5), DNA repair proteins (XRCC5, XRCC6 and PRKDC), an oxygenase (ALKBH5), an ubiquitin-specific protease (USP10), and a phosphatase (DUSP14). Additionally, several proteins encoded by uncharacterized open reading frames (C1orf35, C16orf80, C11orf31, C9orf114, C19orf47) were observed to be RNA binding.

### Over-Representation of Nucleic Acid Binding Domains

Next, we classified the identified proteins based on their three-dimensional structure and amino acid sequence. For the structural classification we first queried the set of mRNA-interacting proteins against the Protein Folding Project database (Drew et al., 2011). This database provided SCOP superfamily classifications derived from sequence similarity (psi-blast), fold recognition and Rosetta de novo structure prediction for the identified RNA-binding proteins. An enrichment analysis of superfamilies showed an over-representation of folds associated with single and double-stranded RNA-binding function (RNA-binding domain “d.58.7”, eukaryotic type KH-domain “d.51.1”, and dsRNA-binding domain-like “b.40.4”), helicases (P-loop containing nucleoside triphosphate hydrolases “c.37.1”) and nucleases (Pin domain-like “c.120.1”) with a corrected p-value 0.05 (Table 1 and Table S3). Interestingly, we also found three structural superfamilies significantly enriched that are associated with DNA binding (HMG-box “a.21.1”, “Winged helix” DNA-binding domain “a.4.5”, and AlbA-like “d.68.6”) suggesting these DNA-binding folds could also interact with RNA. The HMG-box fold is found in high mobility group (HMG) proteins and the structure specific recognition protein 1 (SSRP1). The “Winged Helix” DNA-binding protein is present in a number of RNA helicases. The AlbA-like fold was found in POP7 and in C9orf23. Notably, the AlbA-like superfamily had already previously been suggested to be involved in RNA binding (Aravind et al., 2003).

To obtain an additional perspective of the mRNA-bound proteome associated structures, we performed Pfam and InterPro domain enrichment analysis using the identified proteins. As expected, most of the significantly enriched domains (corrected p-value≧0.05, Table S3) were various RNA-interaction motifs (Table 1, recently reviewed by (Ascano et al., 2011)). Besides the commonly recognized RNA-binding domains, we found an over-representation of several domains with putative RNA-binding activity (Table 1 and Table S3). Among these were the SWAP/SURP domain and the RAP-domain, for which an RNA binding activity was suggested based on sequence comparisons (Denhez and Lafyatis, 1994)(Lee and Hong, 2004). In addition, we found two domains with DNA-binding function (zf-NF-X1 and HMG box) enriched in our analyses.

Finally, to estimate the depth of the mRNA-bound proteome we covered in our oligo(dT) precipitations, we compared, in the absence of a deep HEK293 proteome, the identified proteins to a theoretical set of expressed proteins deduced from mRNA sequencing data. The top 9765 expressed mRNAs make up 95% of the total cellular mRNA molecules surrogating the HEK293 Proteome. We compared the number of mRNA binders encoding at least one specific RNA-binding domain to the number of respective RNA-binding domain containing proteins encoded by the top 9765 expressed mRNAs. FIG. 13 shows that the majority of RNA-binding domain-containing proteins theoretically expressed in HEK293 were identified by our analyses. We could detect 136 out of expressed 164 proteins with an RNA-recognition motif (RRM), 26 out of 28 proteins containing the K-homology (KH) domain, and 4 out of 4 Pumilio domain proteins, which exclusively bind to 3′UTR regions (Quenault et al., 2011).

mRNA-Bound Proteome Connects Posttranscriptional Regulation to DNA-Related Processes

In order to systematically examine the connectivity of the identified mRNA binders and their potential relationship to non-mRNA related biological processes, we generated a network based on protein-protein interaction (PPD. When comparing the PPI-network of mRNA-binders to a random network of equal size we observed a higher average clustering coefficient, indicating the presence of highly interconnected protein-clusters within the network. Because these clusters are indicative of functional modules mediating the regulation of complex biological processes, we analyzed the set of mRNA binders and their first neighbours, based on protein-protein interactions (PPI), for an enrichment of Gene Ontology (GO) terms linked to biological processes (Ashburner et al., 2000). As expected, the most significantly over-represented GO terms were mRNA splicing, localization, processing and translation (Table 2). In addition we observed an over-representation for DNA-related processes, namely “response to DNA damage”, “DNA-dependent transcription”, and “DNA duplex unwinding” (Table 2).

The PPI sub-network for members linked to the term “response to DNA damage” (GO ID 6974) has been generated (not shown). Central to this network are XRCC6/Ku70, XRCC5/Ku80, and the DNA-activated protein kinase (PRKDC). These proteins were identified in each of the three proteomics analyses. Besides their role in DNA double strand break repair and recombination, the proteins have been shown to interact with RNA structures, such as the RNA-stem loop region in yeast telomerase TLC1 and the RNA-component of human telomerase (hTR) (Ting et al., 2005). In addition, XRCC6 had been suggested to bind internal ribosomal entry site (IRES) elements and likely involved in the regulation of IRES-mediated mRNA translation (Silvera et al., 2006). XRCC6 harbors a DNA/RNA-binding SAP-domain, which was a significantly over-represented domain in the mRNA-bound proteome (Table 1).

Besides the identification of several proteins participating in DNA damage response, we observed several protein clusters enriched for additional GO-biological process terms which are not directly connected to RNA metabolism (Table 2), suggesting interplay between posttranscriptional regulation and DNA-related processes in the cell.

Validation of RNA-Binding Function of Several Novel mRNA Binders

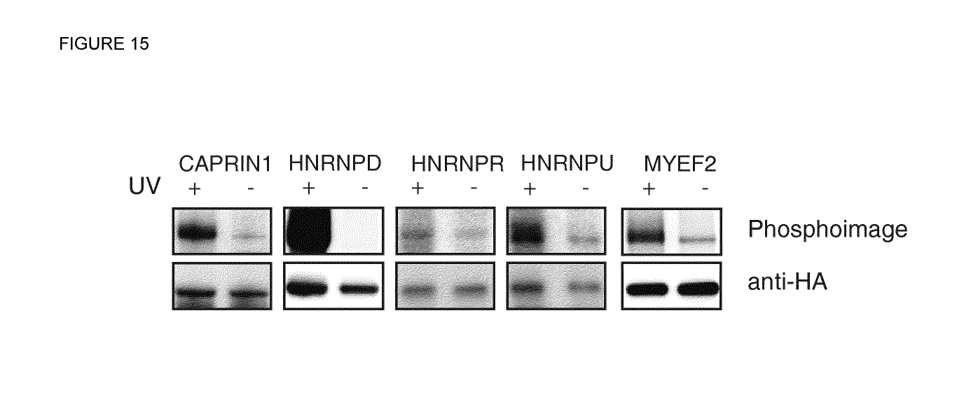

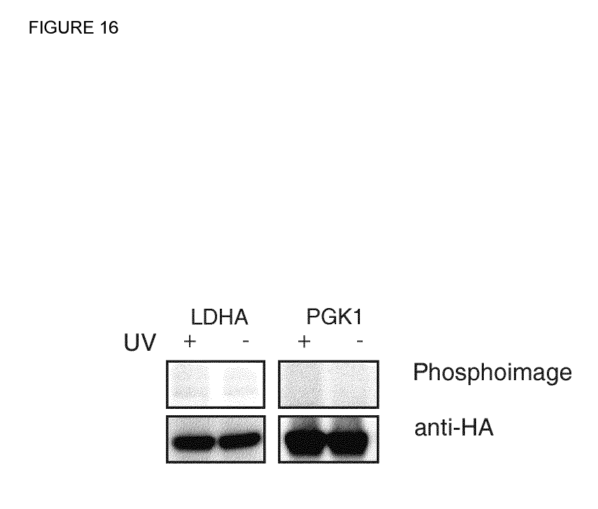

To validate the RNA-binding activity of a subset of the identified proteins, we applied a crosslinking-immunoprecipitation (CLIP) assay. HEK293 cells, stably expressing epitope-tagged mRNA binders, were grown in the presence of 4SU and UV-irradiated at 365 nm. Immunopurified and RNase-treated protein-RNA complexes were radio-labeled using T4 polynucleotide kinase, separated by SDS-PAGE and blotted onto a nitrocellulose membrane. The radio-labeled protein RNA-complexes were visualized by phosphoimaging, whereas protein precipitation was monitored by Western analysis. As positive controls in this CLIP assay, we used five RNA-binding proteins: CAPRINI (Shiina et al., 2005), HNRNPD/AUF1 (Knapinska et al., 2011), HNRNPR (Hassfeld et al., 1998), HNRPNU (Kiledjian and Dreyfuss, 1992), as well as MYEF2, which is a transcriptional repressor (Haas et al., 1995) with an RNA recognition motif (RRM) domain. As expected, the epitope-tagged proteins immunopurified from UV-irradiated cells efficiently crosslinked to RNA, when compared to proteins that were immunoprecipitated from non-irradiated cells (FIG. 15). In contrast, we were unable to detect radiolabeled protein-RNA complexes in immunoprecipitations of phosphoglycerate kinase 1 (PGK1) and lactate dehydrogenase A (LDHA) (FIG. 16), two metabolic enzymes that were not identified in our proteomic analysis as potential RNA-binders (Table S2).

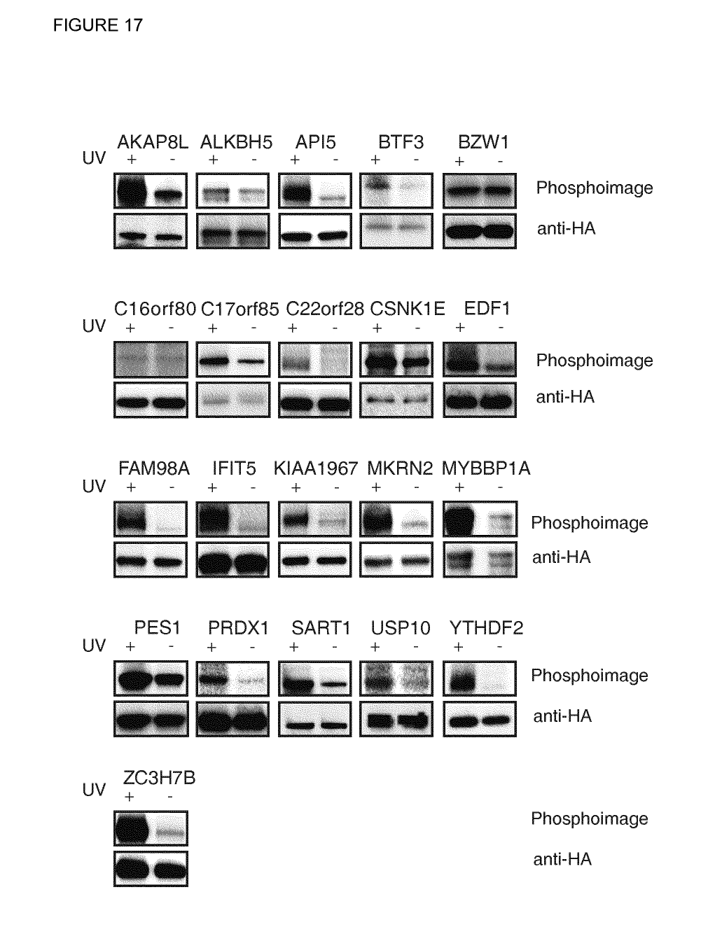

We generated HEK293 cell lines stably expressing 29 putative mRNA-interacting proteins as epitope-tagged versions. 21 proteins could be immunoprecipitated and were used in the crosslinking-IP assay (Table S4). We tested the RNA-binding activity of 18 candidates belonging to group 1 and three members of group 2, BTF3, C16orf80 and PRDX1 (FIG. 17). For all proteins, except BZW1 and C16orf80, we observed an increased radioactive signal in IPs of irradiated cells, indicating these proteins were crosslinked to RNA, and thus directly interact with RNA.

AKAP8L, FAM98A, USP10, SART1, YTHDF2, and ZC3H7B were previously found to be present in complexes containing RNA-binding proteins, suggesting that these proteins themselves can interact with RNA. Interestingly, several of the crosslinked proteins possess enzymatic activities: ALKBH5 (2-oxoglutarate oxygenase), C22orf28 (RNA ligase), CSNK1 E (kinase), MKRN2 (ubiquitin ligase), PRDX1 (peroxidase), and USP10 (ubiquitin thioesterase). Furthermore several of the novel RNA-binding proteins have been implicated in transcriptional regulation either by inhibition of histone deacetylases (KIAA1967) or by acting as transcription factor (BTF3, MYBBP1A, and EDF1). Since the EDF1 encodes a prokaryotic-type helix-turn-helix motif, suggesting this protein may function in DNA binding, we further examined the nature of the crosslinked nucleic acid. When we incubated the immunoprecipitate with RNAse I, but not with DNAse I, the radioactive signal of the ribonuclease-treated complex was reduced, indicating that EDF1 was crosslinked to RNA. In addition, our data indicated that two proteins, C17orf85 and IFIT5, whose molecular functions are unknown, were crosslinked to RNA.

Identification of RNA-Binding Sites of Several Novel mRNA Interactors

To confirm that a subset of our novel identified RNA-binders are indeed binding mRNA transcripts and to identify their binding sites at high resolution, we applied photoactivatable-ribonucleoside-enhanced crosslinking and immunoprecipitation (PAR-CLIP) in combination with next generation sequencing (Hafner et al., 2010). In PAR-CLIP experiments, crosslinking of 4SU-labeled RNA to proteins leads to specific T to C transition events in cDNA sequences, marking the protein binding site on the target RNA (Hafner et al., 2010).

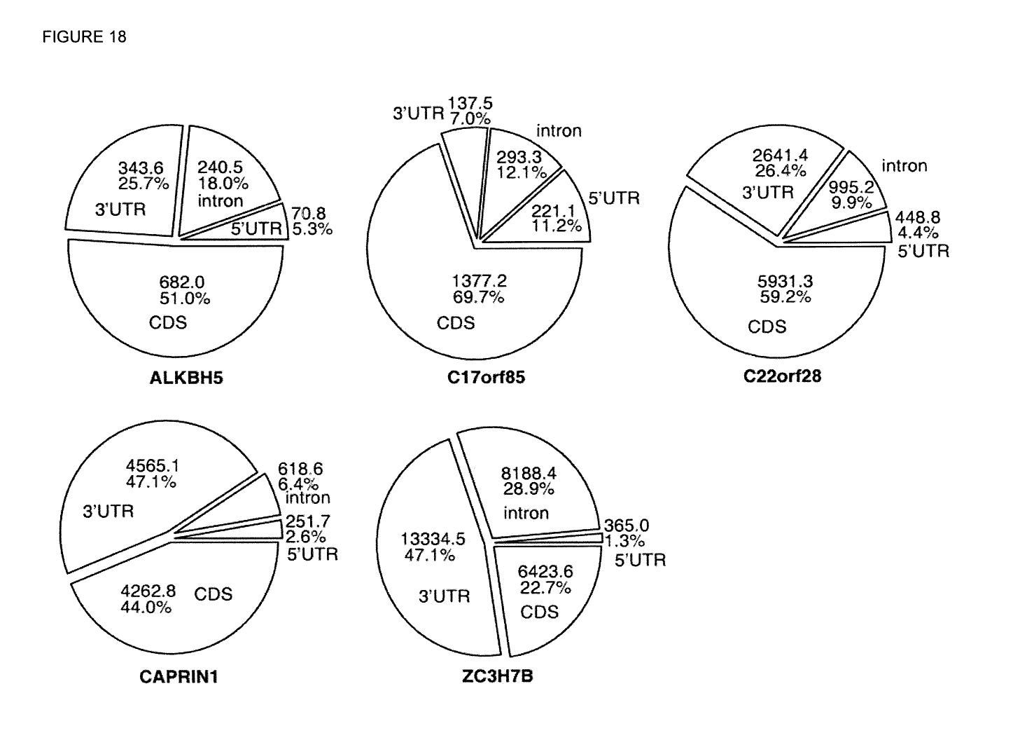

We performed PAR-CLIP experiments for five proteins: ALKBH5, C22orf28, C17orf85 and ZC3H7B, as well as the known RNA-binding protein CAPRINI (Table S5). Diagnostic T to C changes in aligned reads demonstrated efficient RNA-protein crosslinking (FIG. 18). All PAR-CLIP sequencing data (Table S5) were analyzed with a computational analysis pipeline (Lebedeva et al., 2011) to determine the consensus binding sites at an estimated 5% false-positive rate from filtered sequence clusters of aligned reads (see Supplemental Experimental Procedures). The mRNA targets for the respective proteins are listed in Table S5.

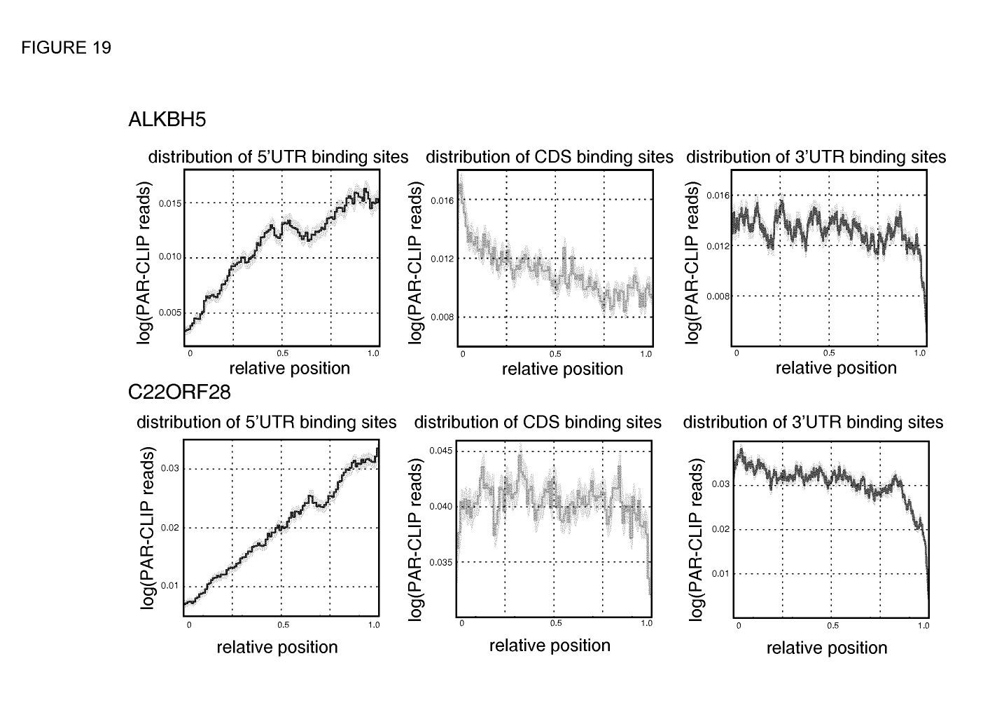

Analyses of PAR-CLIP data confirmed that the five tested proteins all bind predominately mRNA. We used RNA immunoprecipitation (RIP) coupled to RT-PCR to confirm the interactions of these proteins with some of their top mRNA targets as identified by PAR-CLIP (FIG. 19). Although all proteins displayed a preference for mRNA, the distribution of binding sites on protein coding transcript differed. The binding sites of CAPRINI were equally distributed over coding sequences and 3′UTR regions. CAPRINI localizes to stress granules in proliferating cells and was suggested to have a role in mRNA transport and local translational control (Shiina et al., 2005). In addition our data indicated that ZC3H7B has a binding preference for 3′UTRs, but can also interact with sequences in introns and CDSs. ZC3H7B was previously shown to form a ternary complex with the translation initiation factor EIF4G and the Rotavirus nonstructural protein NSP3 in virus infected cells (Vitour et al., 2004).

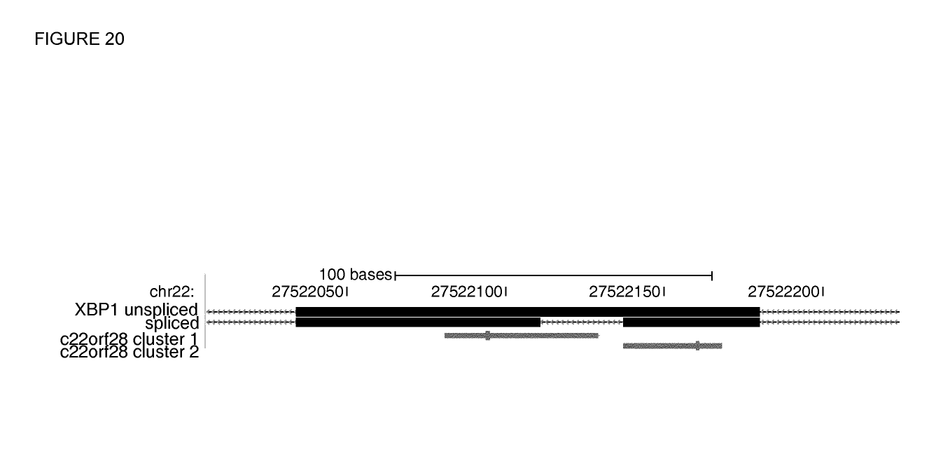

The majority of binding sites of the proteins ALKBH5, C17orf85 and c22orf28 were identified in CDSs. To our knowledge, this is the first time that such a distribution of protein-RNA contacts has been observed. ALKBH5 is 2-oxoglutarate dependent oxygenase and a direct target of hypoxia-inducible factor 1a (HIF-1α) (Thalhammer et al., 2011). In contrast to C22orf28, the ALKBH5 and C17orf85 binding sites were preferentially distributed to the distal 5′ region of CDSs (FIG. 20).

C22orf28, also known as HSPC117, is the essential subunit of a human tRNA splicing ligase complex (Popow et al., 2011). A closer inspection of the C22orf28 target transcripts revealed that the ligase contacts the X-box binding protein 1 (XBP1) mRNA. Interestingly, two of the C22orf28 RNA binding sites in XBP1 are flanking an intron (FIG. 20), which is removed by endoplasmic reticulum stress-induced unconventional cytoplasmic splicing (Yoshida et al., 2001). Our findings suggest that the protein is the elusive ligase in this enzyme-mediated splicing event.

Protein Occupancy Profiling on mRNA Reveals Widespread Binding to 3′UTRs

Present day CLIP data only provides insight into the transcriptome-wide RNA binding sites of close to 30 mammalian RNA interactors (Milek et al., 2011), less than 5% of the 800 mRNA binders identified in this study, leaving the majority of cis-regulatory mRNA elements contacted by these proteins intangible.

Therefore we set out to globally identify the RNA regions that interact with the mRNA-bound proteome by assessing the transcriptome-wide T-C transition profile in cDNA sequences derived from 4SU-labeled RNA crosslinked to all mRNA binders. The crosslinked 4SU residues indicate the RNA contact sites of RNA-interacting proteins and thus should enable us to globally profile the protein occupancy on the mRNA transcriptome.

We generated protein occupancy cDNA libraries for two biological replicates. Briefly, we crosslinked 4SU-labeled cells and purified protein-mRNA complexes using oligo(dT)-beads. The precipitate was treated with RNAse Ito reduce the protein-crosslinked RNA fragments to a length of about 30-60 nt. To remove non-crosslinked RNA, protein-RNA complexes were precipitated with ammonium sulfate and blotted onto nitrocellulose. The RNA was recovered by Proteinase K treatment, ligated to cloning adapters, and reverse transcribed. The resulting cDNA libraries were PCR-amplified and next-generation sequenced (Table S6).

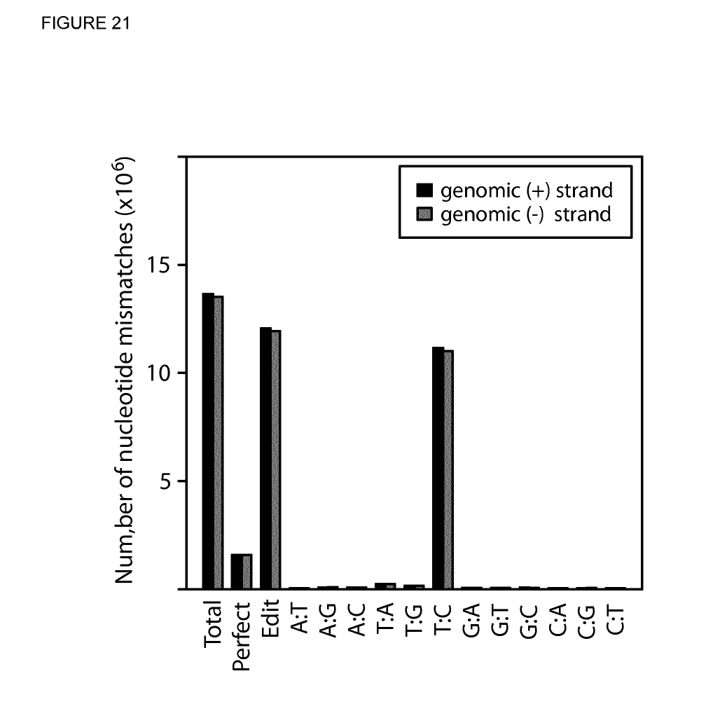

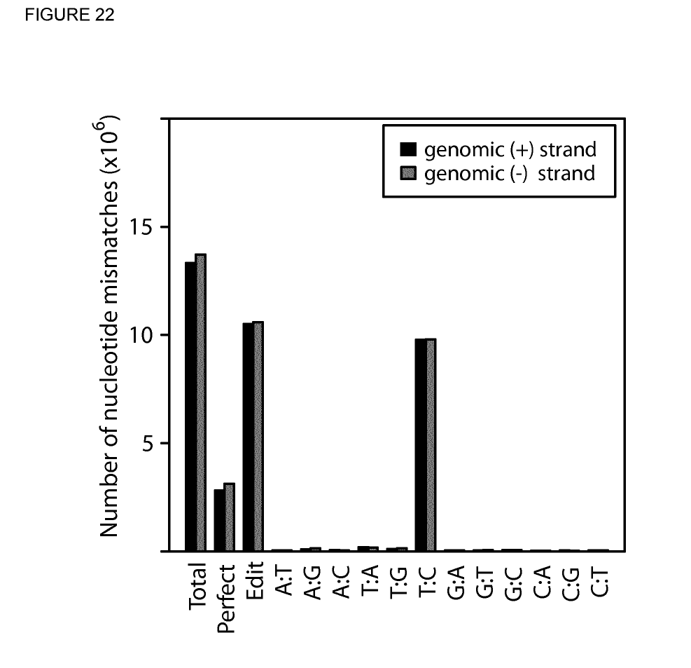

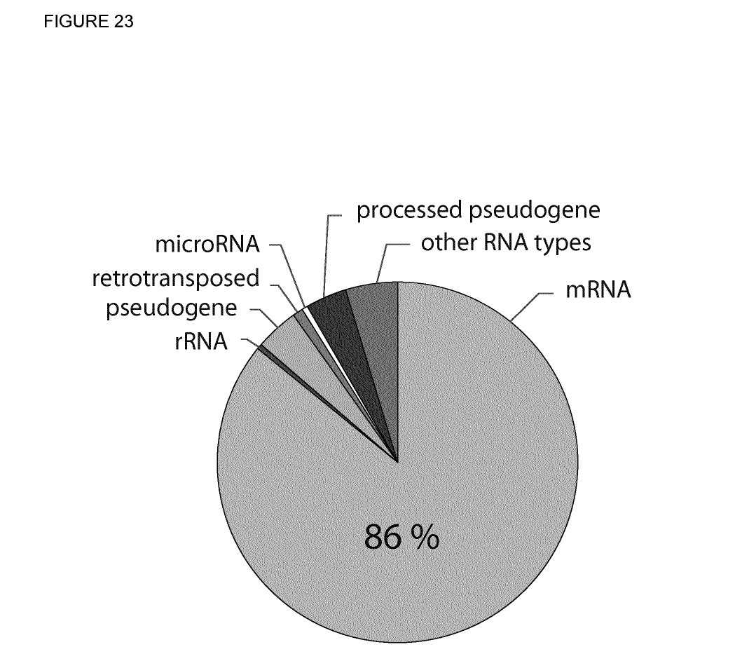

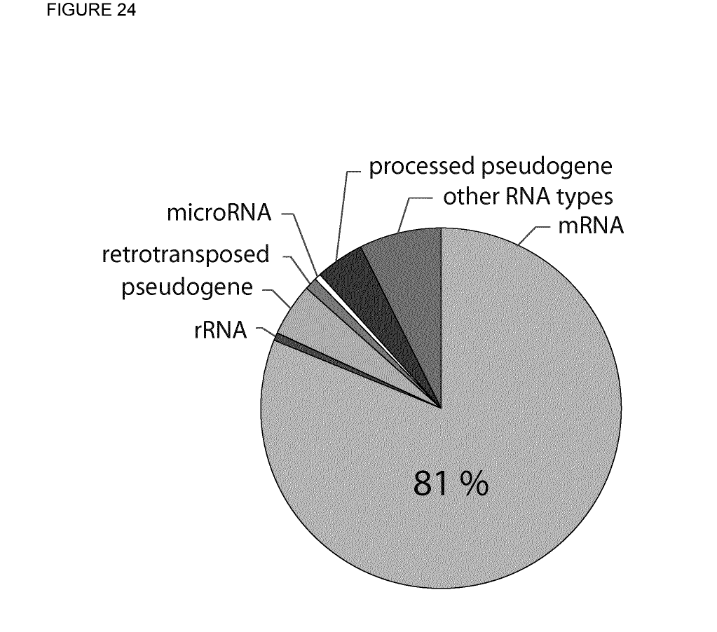

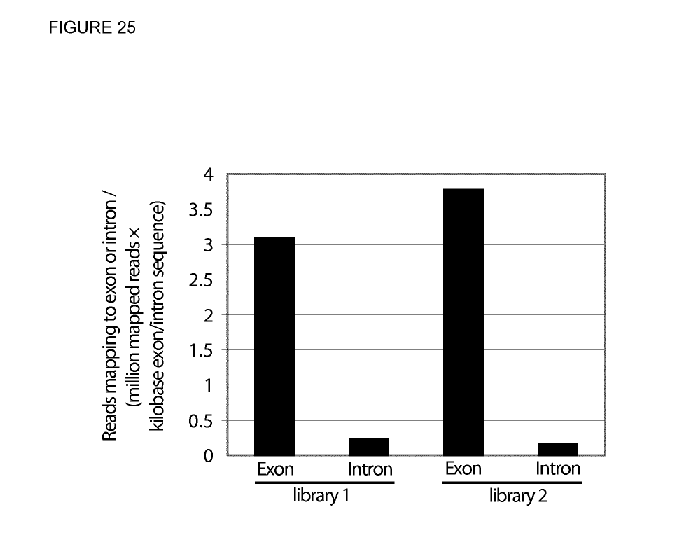

When mapping the sequence reads to the human reference genome, we observed diagnostic T-C changes (FIGS. 21 and 22) for both profiling libraries, indicative for crosslinking of 4SU-containing RNA to proteins (Hafner et al., 2010). The majority of the sequence reads mapped to mRNA sequences (86% and 81%; FIGS. 23 and 24), confirming that the bulk of oligo(dT)-precipitated transcripts were derived from protein-coding genes and therefore the purified proteins predominately bound to mRNA. A comparison of a transcriptome-wide sequence-normalized read count indicated that the proteins preferentially bound exons over introns (FIG. 25).

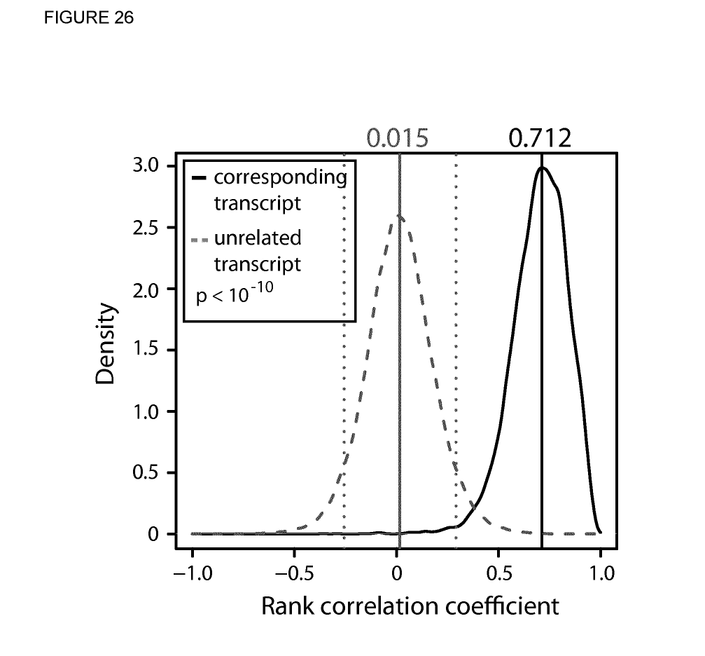

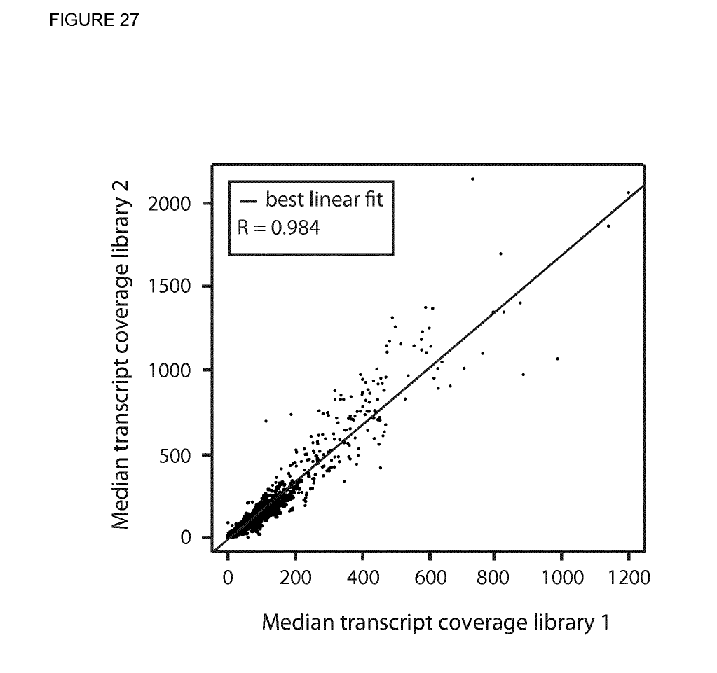

To assess the reproducibility of our approach, we computed rank correlation coefficients for all transcripts using a sliding window approach to compare sequence coverage over entire transcripts. FIG. 26 shows the density distribution of rank correlation coefficients for corresponding transcripts in both experiments (median 0.712) compared to the correlation of randomly selected unrelated transcripts (median 0.015). Next we compared the median coverage over entire transcripts (median of all windows for each transcript) between replicate experiments (FIG. 27) and obtained a rank correlation coefficient of 0.984, suggesting a high degree of similarity between replicate experiments, both in coverage signal for individual transcript regions and overall transcript sequence coverage.

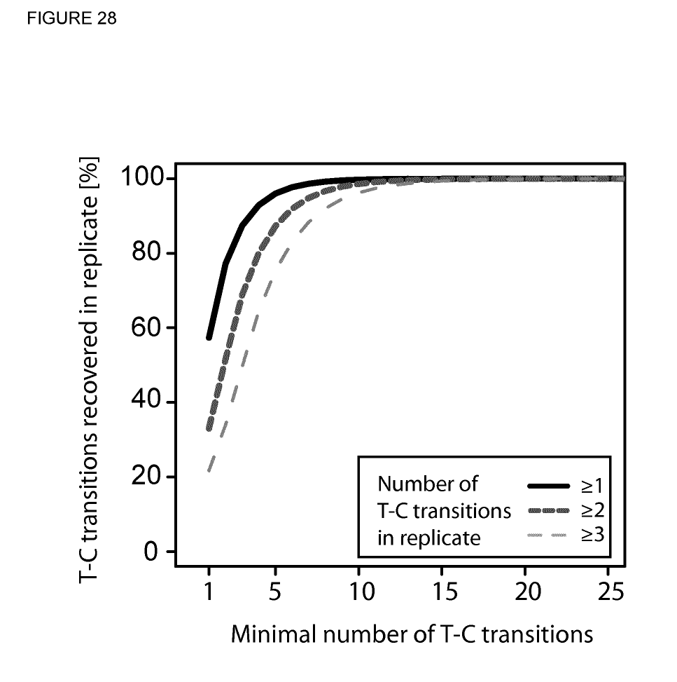

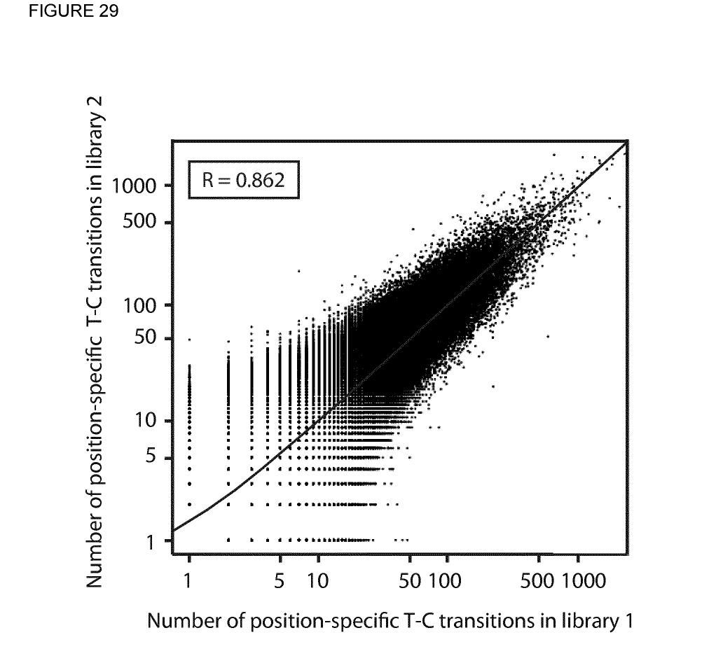

We further analyzed the reproducibility of the occurrence of T-C changes at specific positions and found high agreement between the two profiles (e.g. about 80% of the T-C positions with at least 5 nucleotide changes in one replicate showed at least two transitions in the other experiment (FIG. 28). Finally, we correlated the absolute number of T-C changes at specific positions, considering only sites with at least two transitions in one of the corresponding transcripts, resulting in a high Pearson correlation coefficient of 0.862 (FIG. 29).

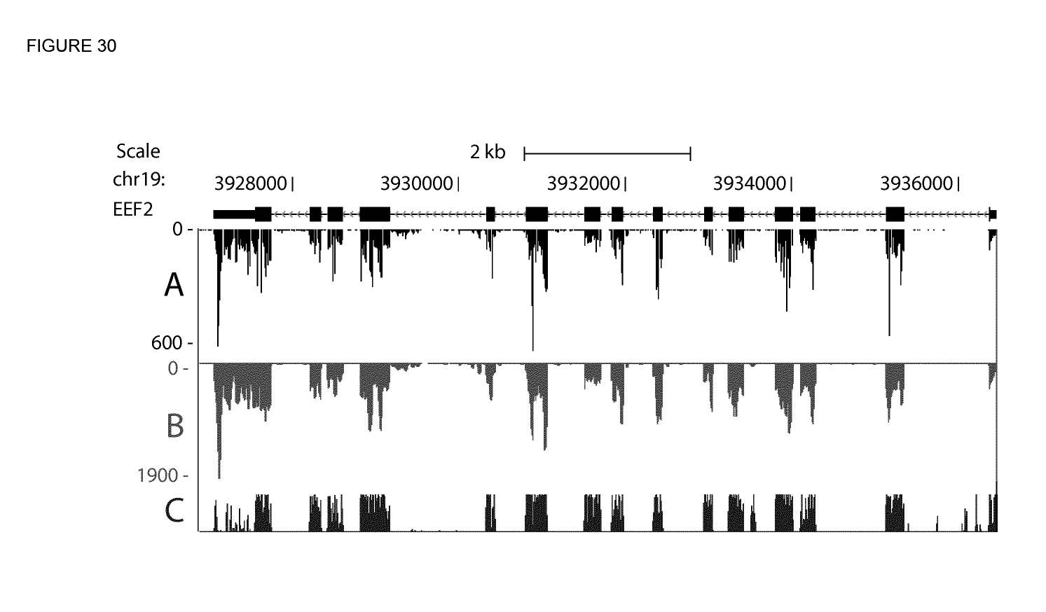

We generated a consensus occupancy profile by using the mean number of T-C changes at positions with at least two T-C changes in each of the two libraries. The transcriptome-wide occupancy profile is available at http://dorina.mdc-berlin.de/cgi-bin/hgTracks (Anders et al., 2011). FIG. 30 shows the consensus T-C transition profile and mean sequence coverage of reads mapping to the genomic region encoding EEF2. As expected T-C changes and sequence coverage were higher in exonic compared to intronic sequences.

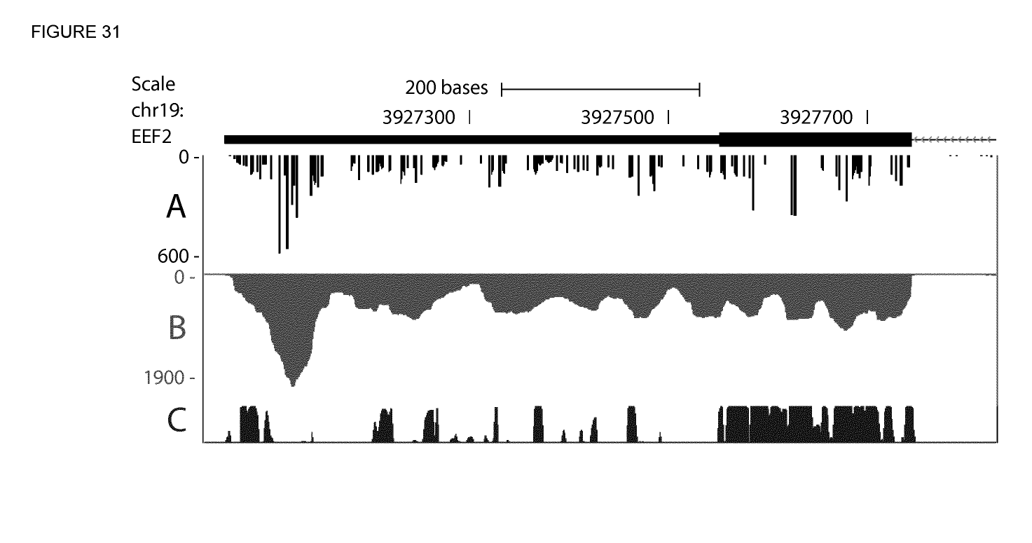

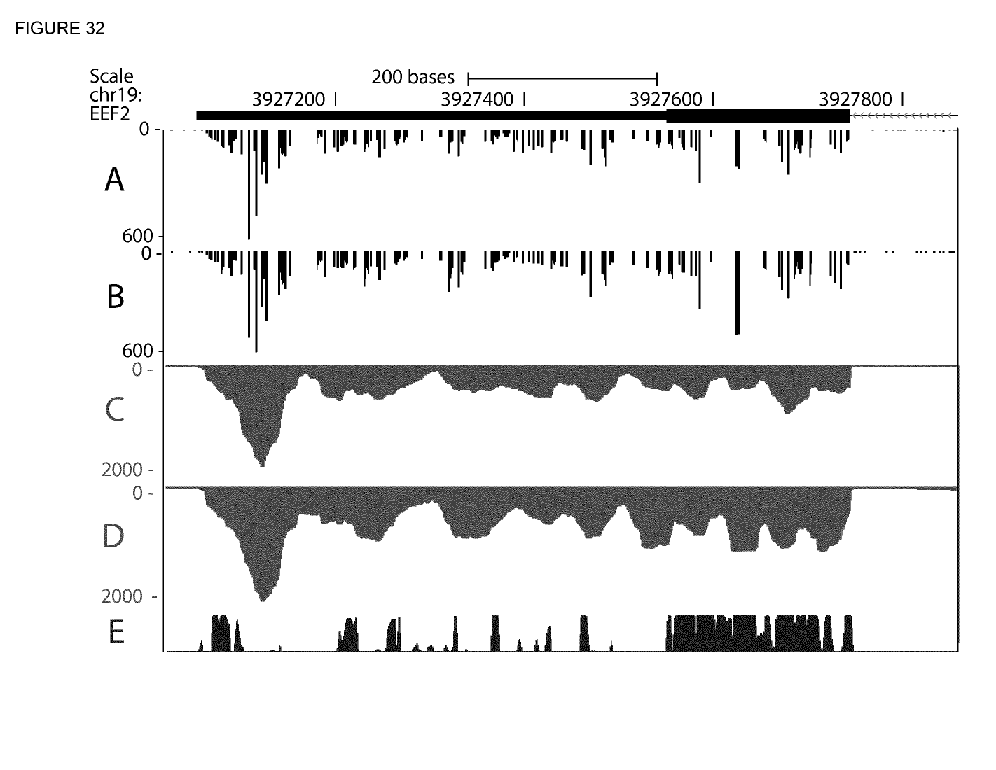

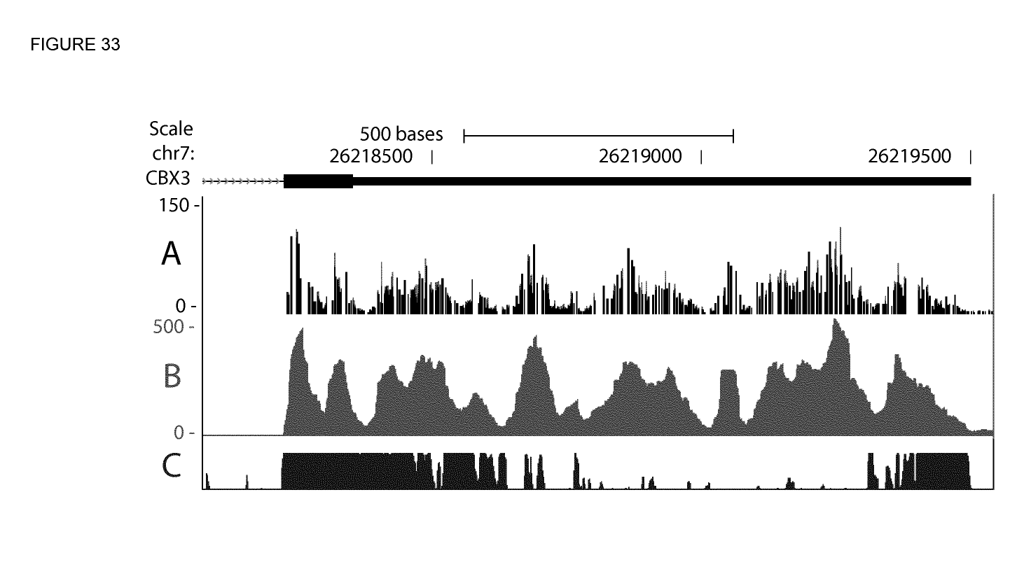

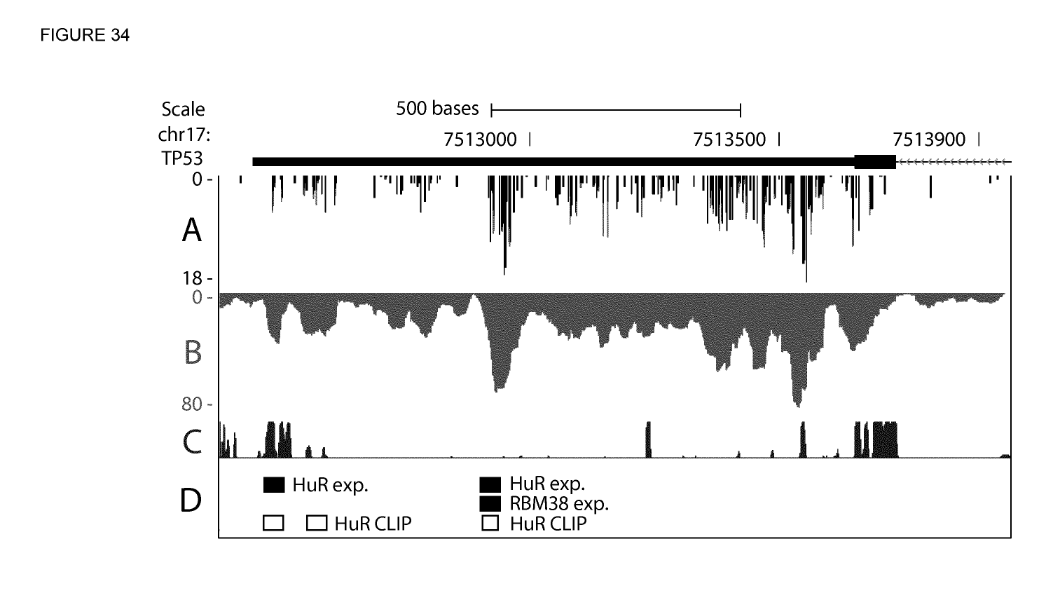

Zooming into the 3′UTR of EEF2 (FIGS. 31 and 32) as well as the 3′UTRs of CBX3 (FIG. 33) and TP53 (FIG. 34) we observed distinct T-C transition profiles indicating regions of protein binding. Intriguingly, three distinct regions with T-C changes in the TP53 3′UTR overlap with previously determined RNA-binding sites, identified either by deletion studies and/or PAR-CLIP experiments (FIG. 34).

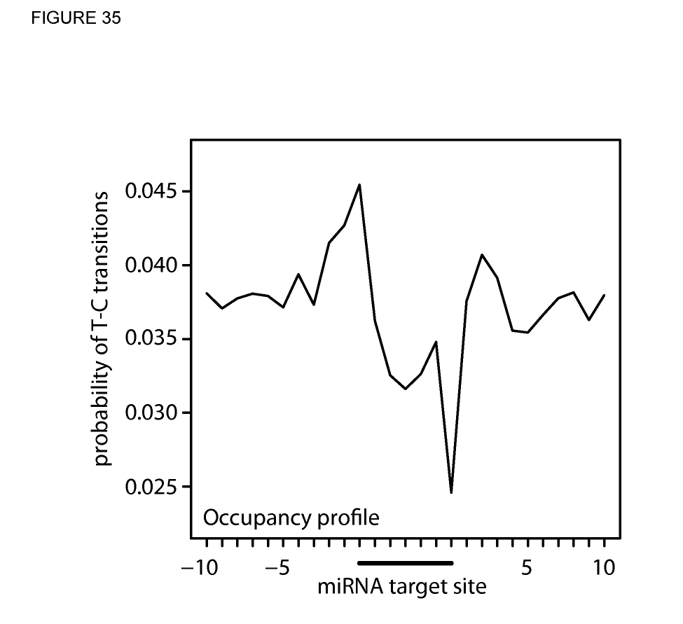

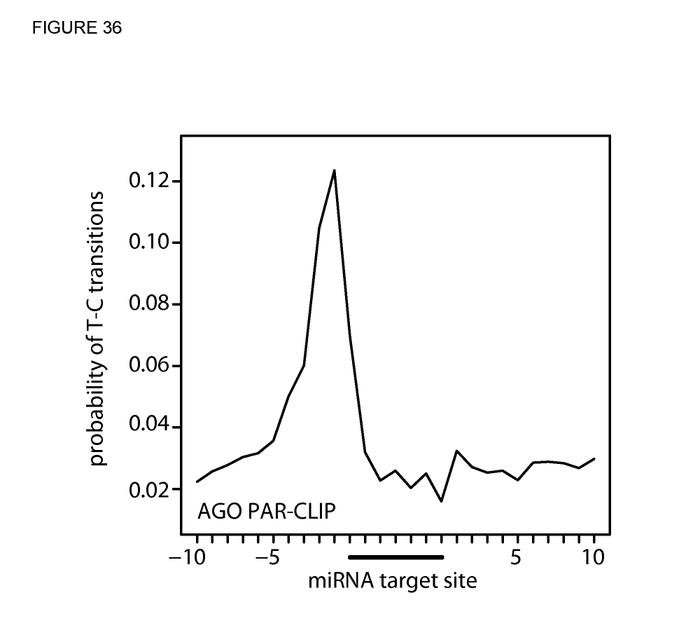

To access whether the occupancy profile indeed reflects binding patterns of RNA interactors, we compared the T-C transition probability around miRNA binding sites in AGO PAR-CLIPs and the occupancy profile. In both cases we observed an increased probability of T-C changes upstream of miRNA binding sites (FIGS. 35 and 36), suggesting that the occupancy profile recapitulates the T-C transition pattern of AGO PAR-CLIPs even in the context of other RNA binders. Furthermore we observed T-C changes in 76% of 32163 AGO binding sites, suggesting that the occupancy profiles encloses the majority of contact sites of this protein.

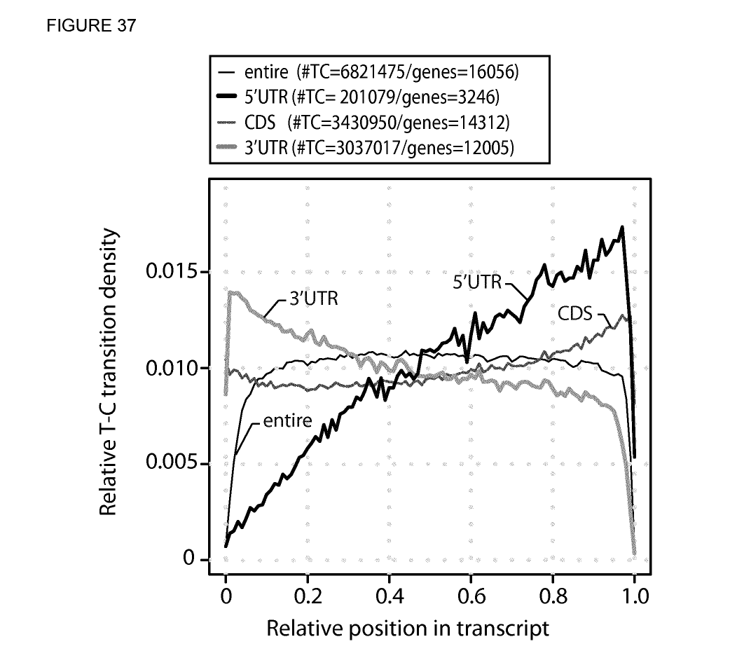

To estimate the general distribution of protein binding to different transcript regions, we averaged the relative density of position with T-C changes of reads mapping to distinct exonic sequences. While protein binding to 3′UTRs was equally distributed, binding in 5′UTRs and CDS showed a preference for 3′ regions (FIG. 37).

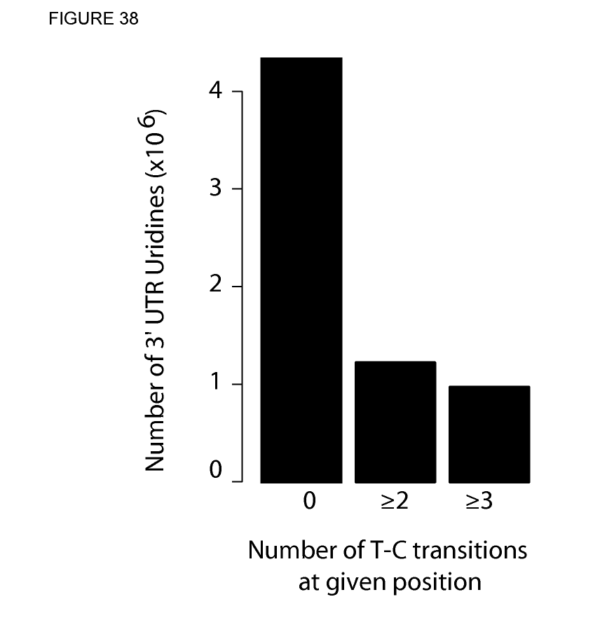

Since we were unable to differentiate whether RNA fragments mapping to mRNA coding sequences were crosslinked to RNA-binding proteins or to translating ribosomes, we further focused our analysis on 3′UTR sequences. The occupancy profiles indicated that extensive regions within 3′UTRs can be bound by proteins. A transcriptome-wide analysis of 3′UTRs showed that 28% of uridines were converted to cytidine (FIG. 38), arguing for widespread protein contacts in this transcript region during the life cycle of polyadenylated mRNAs.

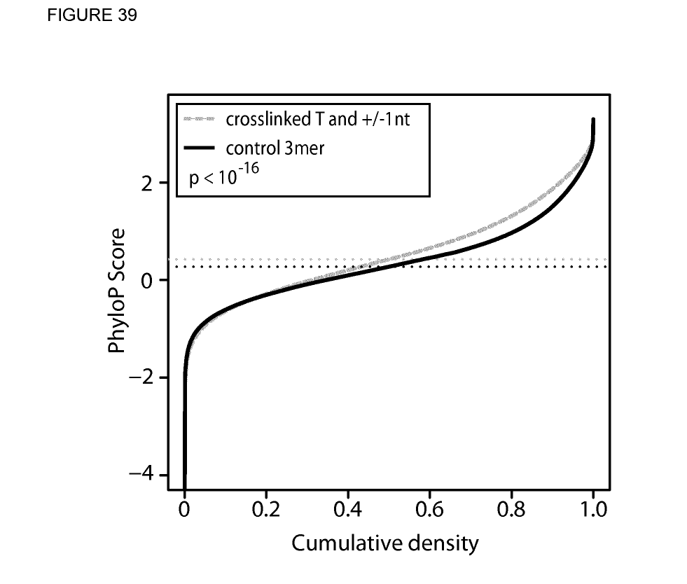

Assuming that the minimal RNA binding region of a protein is at least three nucleotides centered around a crosslinked uridine, we analyzed the evolutionary conservation of such contact sites across 44 vertebrate species and observed a significantly elevated PhyloP conservation score (Pollard et al., 2010) (FIG. 39), suggesting that the crosslinked regions are of functional importance. Next we extended our analysis by examining the density of single nucleotide polymorphisms (SNPs) in minimal RNA binding regions centered around positions with T-C changes. Crosslinked regions showed a significantly lower SNP frequency compared to non-crosslinked control regions (T-C=0.004106, non-T-C=0.005663, binominal test: p-value <2.2e-16), suggesting that sites with T-C changes are under stronger negative selection in humans further supporting their functional relevance.

Putative RNA Cis-Regulatory Elements with Trait/Disease-Associated Polymorphisms

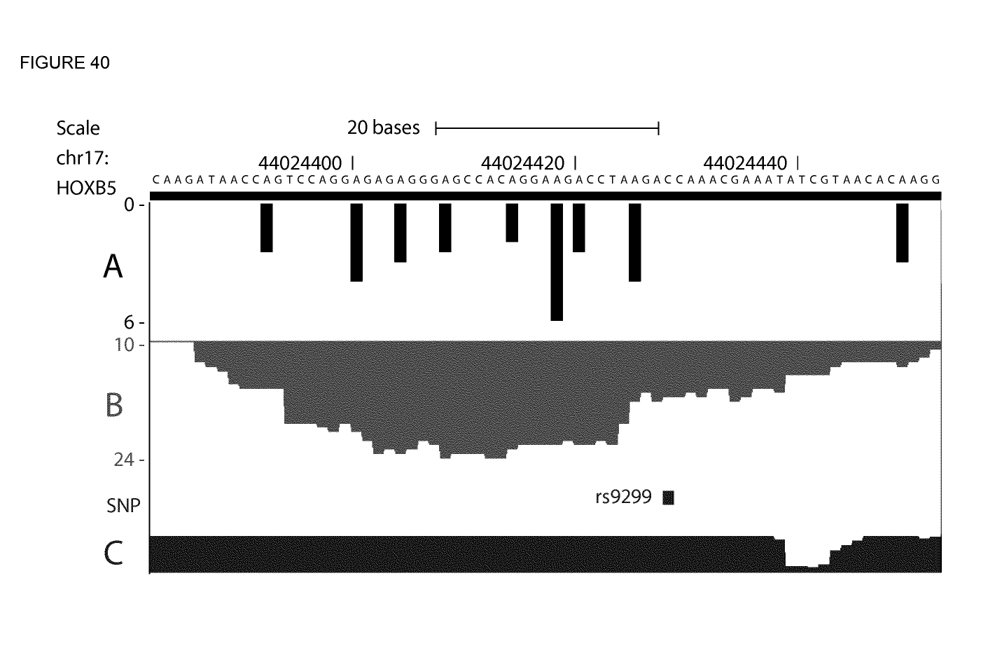

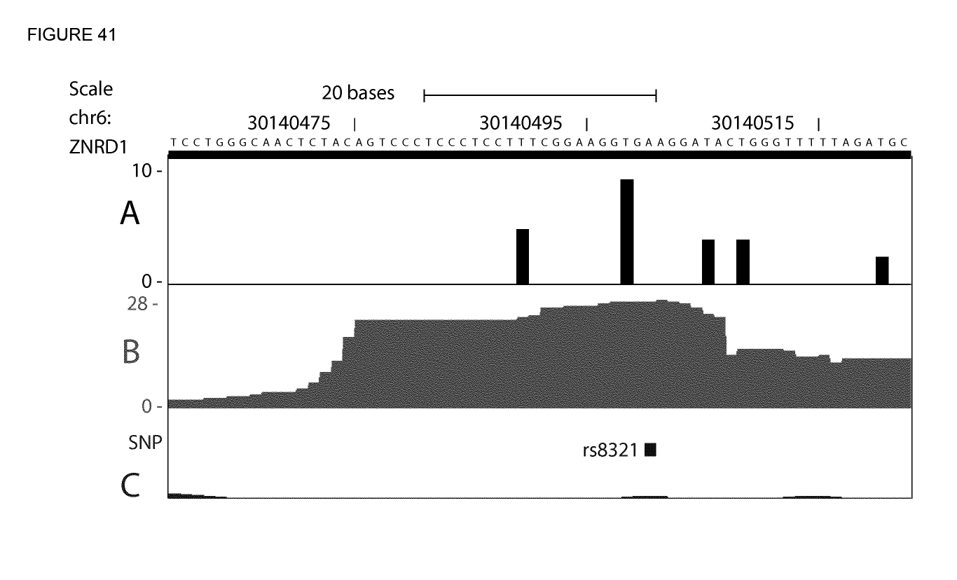

SNPs occurring in binding sites of RNA-interacting proteins could be a contributing factor to cis-modulation of gene expression by changing the affinity of a regulatory protein to untranslated RNA regions. We examined trait/disease-associated SNPs (TASs), obtained from a listing of genome-wide association studies (Hindorff et al., 2009), for their presence in potential RNA binding sites. We focused on TASs within 10 nt around crosslinking site. In total, we identified 28 TASs within potential protein binding sites in introns and 3′ UTRs of mRNAs as well as intergenic regions (Table S7). As shown in FIGS. 40 and 41, rs9299 and rs8321 are TASs that are located in the 3′UTRs of HOXB5 and ZNRD1, respectively. rs9299 has been reported to be linked to childhood obesity (Bradfield et al., 2012),while rs8321 was described to be associated with AIDS progression (Limou et al., 2009).

Short Description of Further Experiments Demonstrating Potential Functional Consequences on (m)RNA Processing.

The present method is associated with unexpected advantages and delivers novel results in light of the prior art.

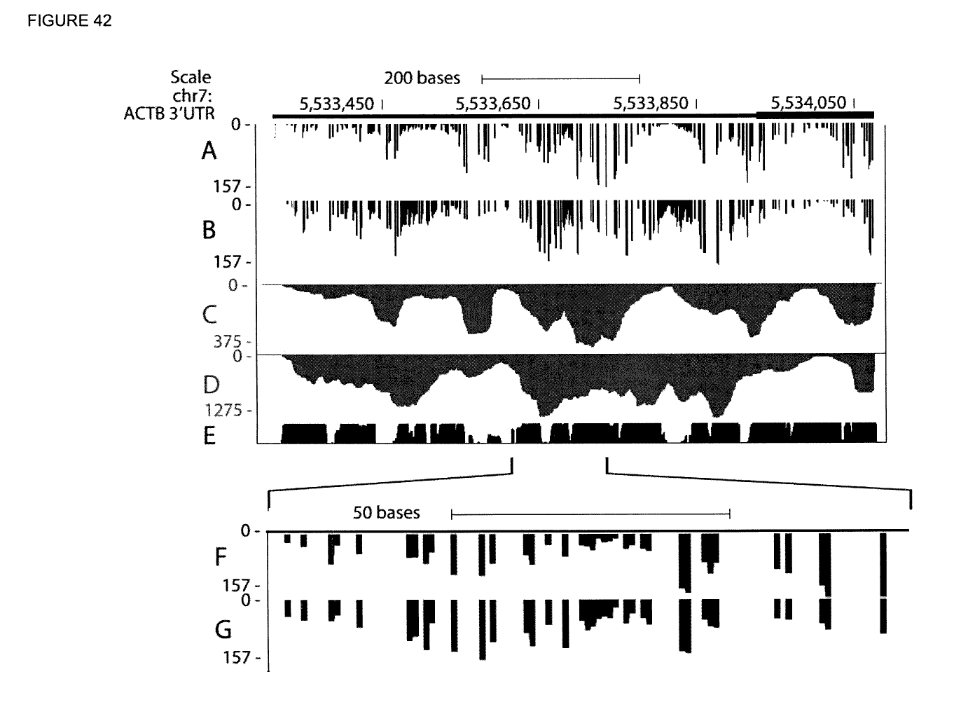

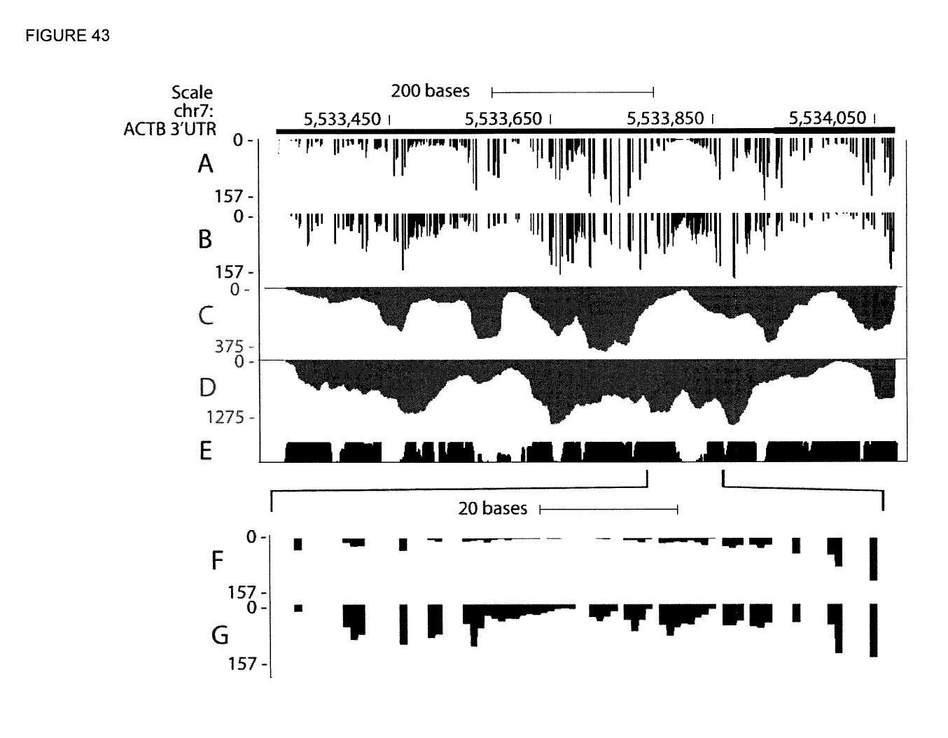

Differential protein occupancy profiling in human MCF7 breast cancer cells and HEK293 human embryonic kidney cells has been carried out using the method of the present invention. As is demonstrated in FIGS. 42 and 43 changes can be observed in particular regions, indicating potentially relevant functional consequences on (m)RNA processing.

The present invention also offers an unbiased search for differentially occupied regions, via crosslinking by RNA-binding proteins rather than ribosomes.

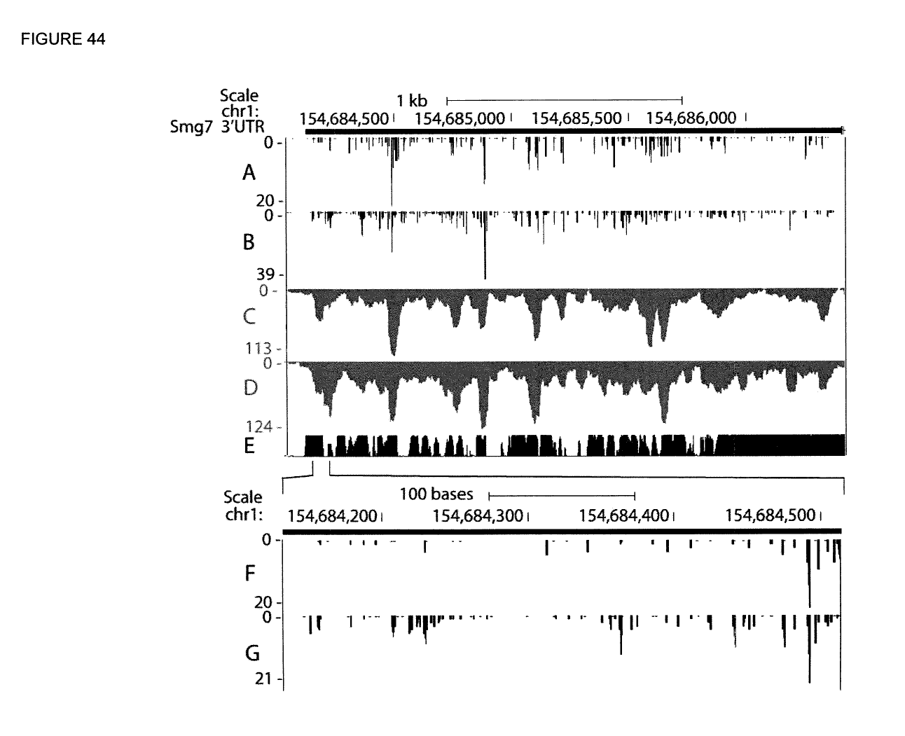

Differential protein occupancy profiling has also been carried out in undifferentiated and differentiated mouse embryonic stem cells. The method provides an analysis of the role of cis-regulatory RNA sequence elements and trans-acting RNA-binding proteins (RBP) that effect post-transcriptional regulation in the context of self-renewal and cell fate decisions. A protein occupancy profiling approach of present invention enables determination of differentially bound regions in undifferentiated and differentiated mouse embryonic stem cells (see FIG. 44). The observations obtained by this approach shed light on mechanisms by which RNA-protein interactions provide the highly selective control of basic cellular processes needed for development and differentiation. In addition, the knowledge of critical RNA-based network modules might facilitate the development of more rational pluripotent cell-based differentiation strategies for treating diseases.

### SUMMARY OF EXPERIMENTAL EXAMPLES

Maturation, localization, decay and translational regulation of mRNAs involve the formation of complexes of RNA-interacting proteins with their target transcripts (Martin and Ephrussi, 2009; Moore and Proudfoot, 2009). Here, we present an approach to characterize the protein-mRNA interactome of a human cell line, based on in vivo UV-crosslinking of proteins to mRNA followed by oligo(dT) affinity purification. The combination of mass-spectrometry-based identification of mRNA-binding proteins and the profiling of their occupancy on RNA by next-generation sequencing significantly expands the ability to define and investigate the protein-mRNA interactome. Recent studies aimed at identifying mRNA-binding proteins in yeast (Scherrer et al., 2010; Tsvetanova et al., 2010), but this is the first study to obtain a comprehensive catalog of proteins interacting with mRNA in human cells.

Using quantitative proteomics we identified around 1236 proteins, which were isolated based on their ability to crosslink to thionucleotide-labeled polyadenylated RNA. SILAC-based proteomics allowed us to quantify the enrichment of proteins in oligo(dT) precipitations from UV-irradatiated cells to a non-irradiated control population. After applying stringent enrichment cutoff criteria we ended up with a list of 801 proteins highly enriched in oligo(dT)-precipitations from UV-irradiated cells.

Sequencing of RNA in the oligo(dT) precipitate and RNA crosslinked to the co-purified proteins showed that the majority of transcripts were derived from protein-coding genes. Close to 90% of the identified proteins were observed in at least two mRNA pulldowns of crosslinked cells compared to those of non-crosslinked cells. As expected a majority, about 70%, of the mRNA binders were proteins previously described to interact with RNA based on their function as RNA-binding proteins, helicases, nucleases and RNA-modifying enzymes. In addition to known RNA-binding domains, our analyses on the enrichment of structural folds and domains revealed several unexpected structures among the identified mRNA binders. In particular, we observed an enrichment of domains found in proteins with DNA binding function, namely the zinc-finger domain, zf-NF-X1, the HMG box, the “Winged helix” DNA-binding domain and the AlbA-like domain. In addition, we observed an overrepresentation for SWAP/SURP and RAP domains, suggesting these domains may also function in RNA binding. Whether any of these domains directly mediate RNA-binding has yet to be investigated, but their significant enrichment makes them excellent candidates for further studies.

Our systematic approach to identify novel mRNA binders resulted in several unexpected findings. Based on our observations we propose a novel RNA binding function for about 260 proteins. These proteins had previously not been shown to interact with RNA nor have recognizable RNA interaction domains, indicating the need for experimental methods to discover novel RNA binders like the one presented here.

The mRNA-interacting proteins also provide interesting insights into how posttranscriptional regulation is connected to other cellular pathways and regulatory mechanisms. In particular transcription seems to be tightly coupled to the subsequent RNA metabolism. Several proteins, for which we confirmed their RNA-binding activity, were shown to function in transcriptional regulation. KIAA1967, also known as Deleted in Breast cancer 1 (DBC1), was initially identified as an inhibitor of the histone acetyltransferase SIRT1 (Kim et al., 2008). Recent work showed that KIAA1967 and SIRT1 play reciprocal roles as major regulators of estrogen receptor a activity (Ji Yu et al., 2011). Initial PAR-CLIP results showed that KIAA1967 directly interacts with mRNA sequences (unpublished). Another new RNA binder is the Myb-binding protein 1a (MYBBPIA). MYBBPIA interacts with and regulates the activity of several transcription factors, including c-Myb (Favier and Gonda, 1994), and NFκB (Owen et al., 2007). Likewise EDF1, also identified as RNA-binding, interacts with the basic leucine zipper proteins, ATF1, c-Jun, and c-Fos, and acts as transcriptional coactivator (Kabe et al., 1999). It is presently unknown by what mechanism these proteins modulate transcription and whether the RNA binding function is required for this activity.

Recent studies identifying RNA-binding proteins in yeast revealed a large number of cytoplasmic proteins with catalytic activities (52 out of 180 identified proteins), many of them acting in metabolism (Scherrer et al., 2010; Tsvetanova et al., 2010). In contrast, we only identified eleven metabolic enzymes among the 801 experimentally determined proteins (Table S2). Still, we discovered a number of non-metabolic enzymes. Among them were C22orf28 and ALKBH5, two proteins that possess catalytic activities previously not found to be associated with mRNA binders. Our findings suggest that C22orf28 is the elusive RNA ligase involved in the cytoplasmic nuclease-mediated splicing of the XBP1 mRNA. On the other hand ALKBH5, found only in vertebrates, possibly functions in oxidative RNA demethylation, since it shows similarity to the Escherichia coli DNA-methylation repair enzyme AlkB and possesses 2-oxoglutarate oxygenase activity (Thalhammer et al., 2011). Interestingly, our set of mRNA binders also included the methyltransferase, NSUN2. Despite its narrow substrate range, catalyzing a 5-methylcytosine modification on tRNAs, NSUN2 might have a broader role in mRNA modification as evidenced by a recent finding of widespread occurrence of 5-methylcytosine in human mRNA (Squires et al., 2012). The discovery of ALKBH5, NSUN2, and several other RNA-modifying enzymes (Table S2) suggests that RNA modifications might be more prevalent in mRNA than anticipated. Further experiments are needed to examine the RNA substrates of these enzymes and their impact on posttranscriptional regulation.

Complementing the identification of the mRNA-bound proteome, we were able to determine the mRNA regions that can crosslink to proteins in HEK293 cells. To our knowledge this is the first time that transcriptome-wide protein binding patterns on mRNAs are being reported. One of the most interesting outcomes was that, during the life cycle of an mRNA molecule, widespread regions of the 3′UTRs provide sites for RNA-binding proteins. About 20% of all thymidines present in 3′UTRs showed more than one diagnostic T to C transition in the protein occupancy profiling sequence reads. This number is reasonably high, considering observations that typically only one of few thymidines in RNA binding sites, when substituted by 4SU, crosslinks to proteins (Hafner et al., 2010). The evolutionary conservation of crosslinked sites suggests that the identified protein-bound RNA segments are of functional importance. In the future a central task will be to overlap occupied region with evolutionary constrained sequences and RNA candidate structures (Lind blad-Toh et al., 2011) as well as with RNA interaction data of individual proteins, to identify specific regulatory elements and their structural contexts.

Our results support the view that transcripts are generally bound and regulated by multiple RNA-interacting proteins (Keene, 2007). The combinatorial assembly of cis-regulatory factors, which takes place in a spatial and time-resolved manner, determines the fate of an mRNA molecule. Untranslated regions of protein-coding transcripts seem to provide ample sequence elements for proteins to bind and to function in the regulation of mRNA biogenesis, localization, decay and translation. Until now, comprehensive high resolution mapping of protein-RNA interactions using different CLIP approaches lead to the discovery of sites of protein-RNA interactions that control distinct posttranscriptional processes. However, these studies focused on the binding specificity and function of single RNA-binding proteins (Hafner et al., 2010; Konig et al., 2010; Ule et al., 2003). Conversely, protein occupancy profiling offers an unbiased view on the transcriptome-wide interactions of the mRNA-bound proteome.

Additionally, the presented protein occupancy profile on mRNA narrows the genomic sequence search space for cis-regulatory elements in untranslated mRNA regions. As our data indicated, the identification of occupied mRNA sites will be very valuable for the examination of rapidly emerging data on genetic variation between individuals. Some polymorphic variations within a population possibly contribute to complex traits and diseases by impacting posttranscriptional and/or translational regulation of gene expression.

In summary, the identification of the mRNA-bound proteome and its occupancy profile on protein-coding transcripts offers a systems-wide view on the protein-mRNA interactome, describing its components and the RNA sites of interactions. Using this approach in the future will greatly contribute to a better understanding of cellular functions of mRNP complexes with the goal to elucidate the posttranscriptional regulatory code that defines growth, differentiation and disease.

### Experimental Procedures

### Oligonucleotides, Plasmids and Antibodies

All oligonucleotides, plasmids and antibodies are described in the Supplemental Information. Plasmids are made available through Addgene (www.addgene.com).

### Cell Culture and Transfection

Human embryonic kidney (HEK) 293 cell lines that allow stable inducible expression of His/FLAG/HA-tagged proteins were generated using the Flp-In System (Invitrogen). For mass spectrometry, cells were grown in SILAC medium as described in (Ong et al., 2002).

### Digital Gene Expression Analysis

mRNA was isolated from TRIzol extracted total RNA using oligo(dT) Dynabeads (Invitrogen) as recommended by manufacturer or by direct precipitation from cell lysates as described for the isolation of mRNA-bound proteins. 4SU- and 6SG-containing RNA was further isolated from non-crosslinked RNA by biotinylation followed by streptavidin-pulldown as described in (Dolken et al., 2008) and As below. The cDNA libraries were generated from each RNA precipitation following the protocol provided by Illumina and the libraries were sequenced on an Illumina GAII by a 1×36 bp run.

Isolation of mRNA-Interacting Proteins

HEK 293 cells were grown for 16 hr in medium supplemented with 4-thiouridine and 6-thioguanosine to final concentrations of 200 μM each. An additional labeling pulse with 100 μM of each photoreactive nucleoside was applied 2 hr prior to UV-irradiation to ensure the labeling of short-lived transcripts. Living cells, grown on light SILAC medium, were irradiated with 365 nm UV light (0.2 J/cm2) whereas the control cells, grown on heavy SILAC medium were not UV-crosslinked (experiment L1 and L2). In label swap experiment (experiment H1), the cells grown on heavy SILAC medium were crosslinked and the cells grown on light SILAC medium were used as control. After crosslinking, cells were harvested and lysed in 10 cell pellet volumes of lysis/binding buffer (100 mM Tris HCl, pH 7.5, 500 mM LiCl, 10 mM EDTA pH 8.0, 1% (w/v) LiDS, 5 mM EDTA, 5 mM DTT, Complete Mini EDTA-free protease inhibitor (Roche). Oligo(dT) beads were added to cell extract and incubated for 1 hr at room temperature on a rotating wheel. The supernatant was saved for further precipitation rounds. Beads were washed with lysis/binding buffer followed by washing and resuspension in NP40 lysis buffer (50 mM Tris HCl, pH 7.5, 140 mM LiCl, 2 mM EDTA pH 8.0, 0.5% NP40, 0.5 mM DTT). Protein-mRNA complexes were eluted from beads in elution buffer (10 mM Tris HCl, pH 7.5) for 2 min at 80° C. For mass spectrometry the RNA was removed by incubation with RNAse I (10 U/ml) and benzonase (125 U/ml) for 3 hr at 37° C. in elution buffer containing 1 mM MgCl₂. After nuclease treatment, the protein solutions were combined and precipitated with trichloroacetic acid, washed with acetone and dissolved in SDS-PAGE loading buffer before separation on a NuPAGE Novex 4 to 12% gradient gel (Invitrogen) followed by in-gel trypsin-digest. Digested protein samples were prepared for mass spectrometry analysis as described in Supplementary Experimental Procedures.

### Validation of RNA-Binding Activity

Cells, stably expressing His/FLAG/HA-tagged proteins, were labeled with 100 μM 4SU, UV-irradiated and lysed in NP-40 lysis buffer. 4SU-labeled non-irradiated cells were used as control. Immunoprecipitation was carried out with anti-FLAG magnetic beads (Sigma). Beads were treated with Calf Intestinal Phosphatase and 5′-endlabeled using T4 polynucleotide kinase. The crosslinked protein-RNA complexes were resolved on 4%-12% NuPAGE gel (Invitrogen), and the corresponding protein-RNA complexes were analyzed by phosphorimaging and Western blotting.

### PAR-CLIP

PAR-CLIP protocol was performed as described in (Hafner et al., 2010). In brief, cells were labeled with 4-thiouridine, UV-irradiated and lysed. After immunoprecipitation, the protein-RNA complex was radiolabeled and separated on SDS-PAGE. The protein-RNA complex was visualized by phosphorimaging and electroeluted. RNA was isolated by proteinase K digestion and phenol-chloroform extraction. Small RNA fragments were cloned and sequenced on an Illumina HiSeq platform according to the small RNA protocol (Hafner et al., 2008). The 3′ ligation was performed with barcoded 3′ adapters. The PAR-CLIP cDNA sequencing data was analyzed using the PAR-CLIP analysis pipeline (Lebedeva et al., 2011).

Protein Occupancy Profiling on mRNA

Flp-ln HEK293 cells were grown in medium supplemented with 200 μM 4SU 16 h prior to crosslinking. Harvested cells were resuspended in 10 pellet volumes of lysis/binding buffer (100 mM Tris-HCl pH 7.5, 500 mM LiCl, 10 mM EDTA pH 8.0, 1% LiDS, 5 mM dithiothreitol (DTT)). Oligo(dT)25 Dynabeads purification was performed as described above. Protein-RNA complexes were TCA precipitated and RNAse I treated. Following RNAse I treatment protein-RNA complexes were precipitated by ammonium sulfate precipitation. Precipitate was separated on a SDS PAGE and transferred to a nitrocellulose membrane. RNA was extracted from membrane by proteinase K treatment and phenol/chloroform extraction. Recovered RNA was dephosphorylated using calf intestinal alkaline phosphatase. After dephosphorylation RNA was phenol/chloroform extracted, ethanol precipitated and 5′ endlabeled using T4 polynucleotide kinase in the presence [γ-³²P]ATP. Radiolabeled RNA was again phenol/chloroform extracted. Subsequent small RNA cloning and adapter ligations were performed as described previously (Hafner et al., 2010). More detailed description of the entire method is provided in Supplementary Experimental Procedures.

### Supplemental Experimental Procedures

### Antibodies

anti-HA.11 (COVANCE, 16B12), anti-FLAG (SIGMA, F1804), anti-HNRNPK (EPITOMICS, EP943Y), anti-mouse immunoglobulins (DAKO), anti-rabbit immunoglobulins (DAKO)

### Oligonucleotides

Small RNA cloning adapters
(SEQ ID NO. 1)
5′adapter
rGrUrUrCrArGrArGrUrUrCrUrArCrArGrUrCrCrGrArCrGrAr
UrC
3′ barcoded adapters (bar-coded is underlined)
NBC1:
(SEQ ID NO. 2)
AppTCTAAAATCGTATGCCGTCTTCTGCTTG-InvdT
NBC2:
(SEQ ID NO. 3)
AppTCTCCCATCGTATGCCGTCTTCTGCTTG-InvdT
NBC3:
(SEQ ID NO. 4)
AppTCTGGGATCGTATGCCGTCTTCTGCTTG-InvdT
NBC4:
(SEQ ID NO. 5)
AppTCTTTTATCGTATGCCGTCTTCTGCTTG-InvdT
NBC5:
(SEQ ID NO. 6)
AppTCTCACGTCGTATGCCGTCTTCTGCTTG-InvdT
NBC6:
(SEQ ID NO. 7)
AppTCTCCATTCGTATGCCGTCTTCTGCTTG-InvdT
NBC7:
(SEQ ID NO. 8)
AppTCTCGTATCGTATGCCGTCTTCTGCTTG-InvdT
NBC8:
(SEQ ID NO. 9)
AppTCTCTGCTCGTATGCCGTCTTCTGCTTG-InvdT
cDNA amplification (restriction sites are
underlined)
ALKBH5:
(SEQ ID NO. 10)
5′-TTCAGTCGACATGGCGGCCGCCAGCGGCTACACGGACCTGCGTGAG
AAG;
(SEQ ID NO. 11)
5′-CTATTGATGCCAACAGCCTTTCCATC,
PGK1:
(SEQ ID NO. 12)
5′-ATGTCGCTTTCTAACAAGCTGACGCTG;
(SEQ ID NO. 13)
5′-ATAAGAATGCGGCCGCCTAAATATTGCTGAGAGCATCCACCCCAG,
qRT-PCR primers
RNU61:
(SEQ ID NO. 14)
5′-GTGCTCGCTTCGGCAGC;
(SEQ ID NO. 15)
5′-TGGAACGCTTCACGAATTTGC
GAPDH:
(SEQ ID NO. 1666)
5′-AGCCACATCGCTCAGACAC;
(SEQ ID NO. 17)
5′-GCCCAATACGACCAAATCC,
RIP/RT-PCR primer
C22orf28:
(SEQ ID NO. 18)
5′-TCAAGACTATCTGAAGGGAATGG;
(SEQ ID NO. 19)
5′-CAGGGGTTGTGTTGAAGACC
CAPRIN1:
(SEQ ID NO. 20)
5′-GCTAGAGGCTTGATGAATGGA;
(SEQ ID NO. 21)
5′-GAAGGGCGGTAACCATCATA
GPI:
(SEQ ID NO. 22)
5′-CATCAACAGCTTTGACCAGTG;
(SEQ ID NO. 23)
5′-GCCATCAAGCTCAGGCTCTA
MACF1:
(SEQ ID NO. 24)
5′-CCGATTGCATCACAACCAT;
(SEQ ID NO. 25)
5′-TTAGCCCATGTCAGGACCTC
MSH6:
(SEQ ID NO. 26)
5′-GCTGTGCGCCTAGGACAT;
(SEQ ID NO. 27)
5′-CCCTTAATGAATTTATAGAGGAACGTA
PKM2:
(SEQ ID NO. 28)
5′-TCCAGGTGAAGCAGAAAGGT;
(SEQ ID NO. 29)
5′-TTCTTGCTGCCCAAGGAG
RPL22:
(SEQ ID NO. 30)
5′-AAATTGTGCCCTGCGAGTT;
(SEQ ID NO. 31)
5-ATGGGAGCCAAGGTAGGACT

### Plasmids

pDONR vectors were largely obtained from the ORFeome project. pENTR constructs were generated by PCR amplification of the respective coding sequences (CDS) from HEK293 cDNA followed by restriction digest and ligation into pENTR4 (Invitrogen). pDONR and pENTR vectors carrying CDS were recombined into pFRT/TO/His/FLAG/HA-DEST destination vector (Invitrogen) using GATEWAY LR recombinase (Invitrogen) according to manufacturers protocol to allow for doxycycline-inducible expression of stably transfected His/FLAG/HA-tagged protein in Flp-ln T-REx HEK293 cells (Invitrogen) from the inducible TO/CMV promoter.

### Cell Lines and Culture Conditions

HEK293 T-REx Flp-In cells (Invitrogen) were grown in D-MEM high glucose with 10% (v/v) fetal bovine serum, 1% (v/v) 2 mM L-glutamine, 1% (v/v) 10,000 μg/ml penicillin/10,000 μg/ml streptomycin, 100 μg/ml zeocin and 15 μg/ml blasticidin.

Cell lines stably expressing His/FLAG/HA-tagged proteins were generated by co-transfection of pFRT/TO/His/FLAG/HA constructs with pOG44 (Invitrogen). Cells were selected by exchanging zeocin with 100 μg/ml hygromycin. Expression of epitope-tagged proteins was induced by addition of 200 ng/ml doxycycline 15 to 20 h before crosslinking. The expression of His/FLAG/ was assessed by Western analysis using a mouse anti-HA.11 monoclonal antibody (Covance).

For quantitative proteomics, cell were grown in SILAC medium as described in

(Ong et al., 2002).Briefly, Dulbecco's Modified Eagle's Medium (DMEM) Glutamax lacking arginine and lysine (a custom preparation from Gibco) supplemented with 10% dialyzed fetal bovine serum (dFBS, Gibco) was used. Heavy (H) and light (L) SILAC media were prepared by adding 84 mg/l ¹³C₆ ¹⁵N₄ L-arginine plus 146 mg/l ¹³C₆ ¹⁵N₂ L-lysine or the corresponding non-labeled amino acids (Sigma), respectively. Labeled amino acids were purchased from Sigma Isotec.

### Mass Spectrometry

Preparations of Oligo(dT) Precipitated Protein-RNA Complexes for Mass Spectrometry Analysis Using in-Gel Digestion

mRNA-bound proteins were isolated as described in experimental procedures and separated on a NuPAGE Novex 4 to 12% gradient gel (Invitrogen) using reducing conditions. Proteins were fixed in fixative solution (50% methanol (v/v), 10% acetic acid (w/v)) and stained afterwards with the Colloidal Blue staining Kit (Invitrogen). Gel lanes were cut into 12 gel slices which were individually subjected to reduction, alkylation and in-gel digestion with sequence grade modified trypsin (Promega) according to standard protocols (Shevchenko et al., 2006). After in-gel digestion peptides were extracted and desalted using StageTips (Rappsilber et al., 2007) prior to analysis by mass spectrometry.

### HPLC and Mass Spectrometry

Reversed-phase liquid chromatography (rpHPLC) was performed employing a Eksigent NanoLC—1D Plus system using self-made fritless C18 microcolumns (Ishihama et al., 2002) (75 μm ID packed with ReproSil-Pur C18-AQ 3-μm resin, Dr. Maisch GmbH) connected on-line to the electrospray ion source (Proxeon) of a LTQ-Orbitrap Velos mass spectrometer (Thermo Fisher). Peptide samples were loaded onto the column with a flow rate of 250 nl/min followed by sample elution at a flow rate of 200 nl/min with a 10 to 60% acetonitrile gradient over 6 h in 0.5% acetic acid. The LTQ-Orbitrap Velos instrument was operated in the data dependent mode (DDA) with a full scan in the Orbitrap followed by up to 20 consecutive MS/MS scans in the LTQ. Precursor ion scans (m/z 300-1700) were acquired in the Orbitrap part of the instrument (resolution R=60,000; target value of 1×106), while in parallel the 20 most intense ions were isolated (target value of 3,000; monoisotopic precursor selection enabled) and fragmented in the LTQ part of the instrument by collision induced dissociation (CID; normalized collision energy 35%; wideband activation enabled). Ions with an unassigned charge state and singly charged ions were rejected. Former target ions selected for MS/MS were dynamically excluded for 60 s. Total cycle time for one full scan plus up to 20 MS/MS scans was approximately 2 s.

### Processing of Mass Spectrometry Data

Identification and quantification of proteins was carried out with the MaxQuant software package (Cox and Mann, 2008). In essence, the Quant.exe module extracts, re-calibrates and quantifies isotope clusters and SILAC doublets in the raw data files (medium labels: Arg6 and Lys4; heavy labels: Arg10 and Lys8; maximum of three labeled amino acids per peptide; polymer detection enabled; top 6 MS/MS peaks per 100 Da). Generated peak lists (msm-files) were submitted to a MASCOT search engine (version 2.2, MatrixScience) and searched against the IPI human database (v. 3.72) supplemented with common contaminants (e.g. trypsin, BSA). The database was modified in-house to obtain a concatenated target-decoy database as described previously (Elias and Gygi, 2007). Full tryptic specificity was required and a maximum of two missed cleavages and a mass tolerance of 0.5 Da for fragment ions applied. Oxidation of methionine and acetylation of the protein N-terminus were used as variable modifications, carbamidomethylation of cysteine as a fixed modification. Filtering of putative MASCOT peptide identifications, assembly of protein groups and re-quantification was performed with Identify.exe. A minimum peptide length of 6 amino acids was required. False discovery rates were estimated based on matches to reversed sequences in the concatenated target-decoy database. A maximum false discovery rate of 1% at both the peptide and the protein level was allowed. Protein ratios were calculated from the median of all normalized peptide ratios using only unique peptides or peptides assigned to respective protein groups with the highest number of peptides (“Occam's razor” peptides). Only protein groups with at least two SILAC counts (peptide ratios) were kept for further analysis.

### SILAC Proteomics Data Analysis

Fold changes were computed by MaxQuant (Cox and Mann, 2008) for proteins and protein groups in case of ambiguities. We considered only fold changes that were supported by at least three measured peptide ratios in a single experiment or three measured peptide ratios over all three experiments (L1, L2 and H1). The quantified protein groups were associated with NCBI Reference Sequence (Refseq) protein IDs by BLASTing the leading protein of the protein group against the human protein database.

Intensity-Based Absolute Quantification (iBAQ) of Proteins

The MaxQuant software computes protein intensities as the sum of all identified peptide intensities (maximum detector peak intensities of the peptide elution profile, including all peaks in the isotope cluster). Protein intensities were divided by the number of theoretically observable peptides (calculated by in silico protein digestion with a PERL script, all fully tryptic peptides between 6 and 30 amino acids were counted while missed cleavages were neglected). “iBAQ intensities” correlate well with absolute protein abundance and can therefore be used for comparison of protein levels within the experiment (Schwanhausser et al., 2011).

### RNA-Binding Protein Validation Assays and PAR-CLIP

Cells were grown in medium supplemented with 100 uM 4SU for 16 h prior to harvest.

### UV 365 nm Crosslinking

For UV crosslinking, the growth medium was removed completely while cells were still attached to the plates. Cells were irradiated on ice with 365 nm UV light (0.2 J/cm2) in a Stratalinker 2400 (Stratagene) equipped with light bulbs for the appropriate wavelength. Cells were scraped off with a rubber policeman in 2 ml PBS per plate and collected by centrifugation at 800×g for 4 min.

### Cell Lysis and First Partial RNase T1 Digestion

The pellets of cells crosslinked with UV 365 nm were resuspended in 3 cell pellet volumes of NP40 lysis buffer (50 mM Tris HCl, pH 7.5, 140 mM LiCl, 2 mM EDTA, pH 8.0, 0.5% (v/v) NP40, 0.5 mM DTT, complete EDTA-free protease inhibitor cocktail (Roche)) and incubated on ice for 10 min. The typical scale of such an experiment was 3 ml of cell pellet. The cell lysate was cleared by centrifugation at 13,000×g. RNase T1 (Fermentas) was added to the cleared cell lysates to a final concentration of 1 U/μl and the reaction mixture was incubated in a water bath at 22° C. for 10 min and subsequently cooled for 5 min on ice before addition of antibody-conjugated magnetic beads.

### Preparation of Dynabeads Protein G Magnetic Beads

10 μl of Dynabeads Protein G magnetic particles (Invitrogen) per ml cell lysate were washed twice with 1 ml of citrate-phosphate buffer (4.7 g/l citric acid, 9.2 g/l Na2HPO4, pH 5.0) and resuspended in twice the volume of citrate-phosphate buffer relative to the original volume of bead suspension. 0.25 μg of anti-FLAG M2 monoclonal antibody (Sigma) per ml suspension was added and incubated at room temperature for 40 min. Beads were then washed twice with 1 ml of citrate-phosphate buffer to remove unbound antibody and resuspended again in twice the volume of citrate-phosphate buffer relative to the original volume of bead suspension.

### Preparation of ANTI-FLAG M2 Magnetic Beads

20 μl of ANTI-FLAG M2 magnetic beads (Sigma-Aldrich) per ml cell lysate were washed twice with 1 ml of citrate-phosphate buffer and resuspended in one original volume of citrate-phosphate buffer.

### Immunoprecipitation, Second RNase T1 Digestion and Dephosphorylation

10 μl antibody-conjugated Protein G magnetic beads or 20 μl of ANTI-FLAG M2 magnetic beads were added per ml of partial RNase T1 treated cell lysate. Incubation was performed in 1.5 ml microfuge tubes on a rotating wheel for 1 hr at 4° C. Magnetic beads were collected on a magnetic particle collector (Invitrogen). Manipulations of the following steps were carried out in 1.5 ml microfuge tubes. The supernatant was removed from the bead-bound material. Beads were washed 2 times with 1 ml of IP wash buffer (50 mM HEPES-KOH, pH 7.5, 300 mM KCl, 0.05% (v/v) NP40, 0.5 mM DTT, complete EDTA-free protease inhibitor cocktail (Roche)) and resuspended in one volume of IP wash buffer. RNase T1 (Fermentas) was added to obtain a final concentration of 50 U/μl, and the bead suspension was incubated at 22° C. for 8 min, and subsequently cooled for 5 min on ice. Beads were washed 3 times with 1 ml of high-salt wash buffer (50 mM HEPES-KOH, pH 7.5, 500 mM KCl, 0.05% (v/v) NP40, 0.5 mM DTT, complete EDTA-free protease inhibitor cocktail (Roche)) and resuspended in two bead volumes of dephosphorylation buffer (50 mM Tris-HCl, pH 7.9, 100 mM NaCl, 10 mM MgCl2, 1 mM DTT). Calf intestinal alkaline phosphatase (CIP) was added to obtain a final concentration of 0.5 U/μl, and the suspension was incubated for 60 min at 37° C. Beads were washed twice with 1 ml of phosphatase wash buffer (50 mM Tris-HCl, pH 7.5, 20 mM EGTA, 0.5% (v/v) NP40) and twice with 1 ml of polynucleotide kinase (PNK) Buffer (50 mM Tris-HCl, pH 7.5, 50 mM NaCl, 10 mM MgCl2, 5 mM DTT). Beads were resuspended in one original bead volume of PNK buffer.

### Radiolabeling of RNA Segments Crosslinked to Immunoprecipitated Proteins

To the bead suspension described above, γ-32P-ATP was added to a final concentration of 0.25 μCi/μl and T4 PNK (CIP) to 1 U/μl in one original bead volume. The suspension was incubated for 30 min at 37° C. Thereafter, nonradioactive ATP was added to obtain a final concentration of 100 μM and the incubation was continued for another 5 min at 37° C. The magnetic beads were then washed 5 times with 800 μl of PNK Buffer and resuspended in 20 μl of SDS-PAGE Loading Buffer (10% glycerol (v/v), 50 mM Tris-HCl, pH 6.8, 2 mM EDTA, 2% SDS (w/v), 100 mM DTT, 0.1% bromophenol blue).

### RNAse and DNAse Digestion Assay

Protein IP was performed according to the RNA-binding protein validation assay protocol until labeling the γ-32P-ATP RNA-labeling step. After radiolabeling, the beads were washed twice with PNK buffer and resuspended in PNK buffer. The sample was divided in three aliquots and incubated at 37° C. for 30 min with either RNAse I (0.1 U/μl) or DNAse 1 (0.1 U/μl). A control sample was incubated at 37° C. without addition of Nucleases. After incubation, the beads were washed 5 times with 800 μl of PNK Buffer and resuspended in 20 μl of SDS-PAGE Loading Buffer.

### SDS-PAGE and Western Blotting

FLAG beads suspension was incubated for 5 min at 95° C. and vortexed. The magnetic beads were separated on a magnetic separator and the supernatant was loaded used for SDS-PAGE. The gel was analyzed by phosphorimaging. To ensure equal protein loading, the protein-RNA complexes were blotted on a nitrocellulose membrane (Hybond™ ECL™, GE Healthcare) and analyzed by phosphorimaging followed by incubation with anti-HA.11 antibody followed by HRP-conjugated secondary anti-mouse IgG antibody and the tagged protein was visualized using the Amersham™ ECL™ (GE-Healthcare) western blot detection reagent.

Electroelution of Crosslinked RNA-Protein Complexes from Gel Slices

The radioactive RNA-protein complex migrating at the expected molecular weight of the target protein was excised from the gel and electroeluted in a D-Tube Dialyzer Midi (Novagen) in 800 μl SDS running buffer according to the instructions of the manufacturer.

Proteinase K digestion

An equal volume of 2× Proteinase K Buffer (100 mM Tris-HCl, pH 7.5, 150 mM NaCl, 12.5 mM EDTA, 2% (w/v) SDS) with respect to the electroeluate was added, followed by the addition of Proteinase K (Roche) to a final concentration of 1.2 mg/ml, and incubation for 30 min at 55° C. The RNA was recovered by acidic phenol/chloroform extraction followed by a chloroform extraction and an ethanol precipitation. The pellet was dissolved in 10.5 μl water.

cdna Library Preparation and Deep Sequencing

The recovered RNA was carried through a cDNA library preparation protocol originally described for cloning of small regulatory RNAs (Dolken et al., 2008; Hafner et al., 2008). The first ligation step was carried out with a 3′ barcoded adapter (see under oligonucleotides) in 20 μl reaction volume using 10.5 μl of the recovered RNA. The PAR-CLIP libraries were sequenced on an Illumina Genome Analyzer GAII and HighSeq using 1×50BP single read protocol.

### PAR-CLIP Computational Analysis

Illumina PAR-CLIP cDNA sequencing reads were aligned to the human genome assembly (hg18), allowing for up to one mismatch, insertion or deletion. Only uniquely mapping reads were retained. We identified clusters of aligned PAR-CLIP reads continuously covering regions of pre-mRNA sequence based on the condition that a sequence cluster requires sequence coverage from both libraries PAR-CLIP libraries for each protein, whereas a read with T to C conversion is only needed from one of the two libraries (consensus assumption). The number of T to C or G to A mismatches served as a crosslink score. We also assigned a quality score based on the number and positions of distinct reads contributing to the cluster.

As the reads should originate from protein-bound transcripts we regarded clusters aligning antisense to the annotated direction of transcription as false positives. We were thus able to select cutoffs on both scores such as to keep the estimated false positive rate below 5%. After filtering by these cutoffs we expect each remaining cluster to harbor at least one binding site {Lebedeva, 2011 #40}.

### RIP and RT-PCR

Cells were harvested, washed in ice-cold PBS and collected by centrifugation (2000 RCF, 4° C., 10 min). Resulting cell pellets were resuspended in 3 volumes of NP40 lysis buffer (50 mM HEPES-KOH at pH 7.4, 150 mM KCl, 2 mM EDTA, 0.5% (v/v) NP40, 0.5 mM DTT, complete EDTA-free protease inhibitor cocktail) and incubated on ice for 10 min. Lysates were cleared by centrifugation (16,000 RCF, 4° C., 15 min).

1/33 of the total volume was mixed with 3 volumes of TRIzol and 0.2 volumes of chloroform to extract total cellular RNA. Phases were separated by centrifugation (16,000 RCF, 4° C., 10 min.) and RNA was ethanol-precipitated.

The remaining cleared extract was incubated FLAG-conjugated ProteinG Dynabeads (Invitrogen) or ProteinG Dynabeads only and incubated 1 h at 4° C.

Beads were washed 3 times with IP wash buffer (50 mM HEPES-KOH at pH 7.4, 150 mM KCl, 2 mM EDTA, 0.05% (v/v) NP40, 0.5 mM DTT, complete EDTA-free protease inhibitor cocktail), resuspended in one original volume of Proteinase K solution (200 mM Tris-HCl at pH 7.5, 300 mM NaCl, 25 mM EDTA, 2% (w/v) SDS, 0.6 mg/ml Proteinase K) and incubated 20 min at 65° C. RNA was phenol chloroform extracted and ethanol-precipitated. The resulting pellet was dried at room temperature and resuspended in H₂O.

Single stranded cDNAs were synthesized from total RNA with an 18 nt oligo-dT primer using Superscript III reverse transcriptase (Invitrogen) according to the manufacturer's instructions. After reverse transcription to cDNA, the precipitated target transcripts were amplified by PCR, spaming approximately 100-150 nt of an intron-spaming target sequence and analyzed by agarose gel electrophoresis.

### Quantitative Real-Time PCR

Single stranded cDNAs were synthesized from total RNA with an 18 nt oligo(dT) primer using Superscript III (Invitrogen) according to the manufacturer's instructions. Real-time PCR was performed using Power SYBR Green PCR master mix (Applied Biosystem) on the StepOne Real-Time PCR System (Applied Biosystem).

Identification of mRNA-Crosslinked Proteins by Western Blot Analysis

Cell lines stably expressing the protein of interest were induced with doxycycline and grown in the presence of 4SU and 6SG as described above. Crosslinking, cell lysis and mRNA precipitation were performed as described above for oligo(dT) precipitations. Input, supernatant after precipitation and the oligo-dT beads bound precipitate were RNAse treated before TCA-precipitation. The protein was loaded on a 4-12% NuPAGE® Bis-Tris gradient gel (Invitrogen). After Western Blotting, the nitrocellulose membrane was incubated either with anti-HA.11 antibody (for endogenous proteins) or with an antibody against the endogenous protein (here: anti-HNRNPK). HRP-conjugated secondary antibodies were used and the proteins were visualized using the Amersham™ ECL™ western blot detection reagent (GE-Healthcare).

Protein Occupancy Profiling on mRNA

Flp-In HEK293 cells were grown in medium (D-MEM high glucose with 10% (v/v) fetal bovine serum, 1% (v/v) 2 mM L-glutamine, 1% (v/v) 10,000 U/ml penicillin/10,000 μg/ml streptomycin) supplemented with 200 μM 4SU 16 h prior to harvest. For UV crosslinking, culture media was removed and cells were irradiated on ice with 365 nm UV light (0.2 J/cm²) in a Stratalinker 2400 (Stratagene), equipped with light bulbs for the appropriate wavelength. Following crosslinking cells were harvested from tissue culture plates by scraping them off with a rubber policeman, washed with ice-cold PBS and collected by centrifugation (2000 RCF, 4° C., 10 min). Resulting cell pellets were resuspended in 10 pellet volumes of lysis/binding buffer (100 mM Tris-HCl pH 7.5, 500 mM LiCl, 10 mM EDTA pH 8.0, 1% LiDS, 5 mM dithiothreitol (DTT)) and incubated on ice for 10 min. Lysates were passed through a 21 gauge needle to shear genomic DNA and reduce viscosity. Dynabeads Oligo(dT)₂₅ were briefly washed in lysis/binding buffer, resuspended in the appropriate volume of lysate and incubated 1 h at room temperature on a rotating wheel. Following incubation, supernatant was removed and stored on ice for multiple rounds of mRNA hybridization. Beads were washed 2 times in 1 lysate volume lysis/binding buffer, followed by 3 washes in 1 lysate volume NP40 washing buffer (50 mM Tris pH 7.5, 140 mM LiCl, 2 mM EDTA, 0.5% NP40, 0.5 mM DTT). Following the washes, beads were resuspended in 1 ml elution buffer (10 mM Tris-HCl, pH 7.5) and transferred to a new 1.5 ml microfuge tube. Hybridized polyadenylated mRNAs were eluted at 80 degrees for 2 min and eluate was placed on ice immediately. Beads were re-incubated with lysate for a total number of 3 depletions by repeating the described procedure.

Following RNAse treatment (RNAse I, Ambion, 1000) protein-RNA complexes were precipitated by ammonium sulfate precipitation. After centrifugation (16000 RCF, 4° C., 30 min) resulting protein pellets were resuspended in SDS-loading buffer and separated on a NuPAGE 4-12% Bis-Tris gel (Invitrogen). Separated protein-RNA complexes were transferred to a nitrocellulose membrane, desired bands migrating between 15 kDa and 250 kDa were cut out and crushed membrane pieces were Proteinase K (Roche) digested (4 mg/ml Proteinase K, 30 min, 55° C.). Following Proteinase K treatment RNA was Phenol/Chloroform extracted and Ethanol precipitated. Recovered RNA was dephosphorylated using Calf Intestinal Alkaline Phosphatase (NEB, 50U, 1 h, 37° C.). After dephosphorylation RNA was Phenol/Chloroform extracted, Ethanol precipitated and subjected to radiolabeling using Polynucleotide Kinase (NEB, 1000, 20 min, 37° C.) and 0.2 μCi/μl-^{32P} γ-ATP (NEG). Radiolabeled RNA was again Phenol/Chloroform extracted and recovered by ethanol precipitation. Subsequent small RNA cloning and adapter ligations were performed as described in previously (Hafner et al., 2010).

### Sequence Analysis of Oligo(dT) Purified RNA

Standard mRNA Purification (mRNA-Seq)

HEK293 total RNA was extracted using TRIzol reagent (Invitrogen) following the manufacturer's instructions. Briefly, HEK293 cells grown on SILAC medium were harvested as described previously and the pellet was immediately suspended in TRIzol reagent and homogenized. 1 ml chloroform was added to 5 ml TRIzol solution, vigorously mixed and incubated at room temperature. After centrifugation (13,000 g, 5 min, 4° C.) the aqueous phase was transferred to a fresh RNAse-free tube and 1 volume ROTI® phenol/chloroform/isoamyl alcohol (25/24/1, v/v) was added. The sample was mixed vigorously, incubated 5 min at room temperature and centrifuged at 13,000 g (5 min, 4° C.). The aqueous phase was transferred to 1 TRIzol volume isopropanol and precipitated on ice. After centrifugation (13,000 g, 30 min, 4° C.) the pellet was washed with 80% (v/v) ethanol. The pellet was dried at room temperature and resuspended in nuclease-free water. Poly(A)+RNA was purified from total RNA by two rounds of precipitation with oligo(dT) beads (Invitrogen) according to the manufacturer's instructions and resuspended in nuclease-free water.

RNA Oliqo(dT) Purification from 4SU and 6SG Labeled Non-Irradiated Cells (“No UV”)

We isolated mRNA from HEK293 cells grown in SILAC medium with addition of 4SU and 6SG by oligo(dT) precipitation as described for the isolation of mRNA-bound proteins but without UV-irradiation. The isolated mRNA was ethanol precipitated, washed and resuspended in nuclease-free water.

Purification of 4SU- and 6SG-Labeled RNA (“4SU+6SG RNA”) by Biotinylation

mRNA was isolated by direct oligo(dT) precipitation from lysate of HEK293 cells grown on SILAC medium with addition of 4SU and 6SG and without UV-crosslinking. mRNA was ethanol precipitated to remove traces of DTT before biotinylation. Biotinylation and pull-down of labeled RNA using streptavidin-conjugated beads was performed as described previously in (Dolken et al., 2008).

RNA Oliqo(dT) Purification from 4SU and 6SG Labeled UV-Irradiated Cells (“UV”)

mRNA was isolated as described before for the isolation of mRNA-bound proteins, starting from UV-irradiated cells.

After elution from oligo(dT) beads, protein-RNA complexes were proteinase K digested in proteinase K reaction buffer (800 mM GuHCl, 50 mM EDTA, 5% Tween 10, 0.5% Triton-X 100) for 3 h at 55° C. with a final proteinase K concentration of 2 mg/ml. The RNA was recovered by acidic phenol/chloroform extraction and ethanol precipitation and resuspended in nuclease-free water.

cDNA Library Preparation for Transcriptome Sequencing

The RNA obtained by the four precipitation methods described above was analyzed by next-generation sequencing. cDNA libraries were prepared from the recovered RNA, following the mRNA sequencing protocol provided by Illumina. Briefly, poly(A) RNA was fragmented using 5× fragmentation buffer (200 mM Tris-acetate, pH 8.1, 500 mM potassium-acetate, 150 mM magnesium-acetate) by heating at 94° C. for 3.5 min. After ethanol-precipitation, first- and second-strand cDNA synthesis was performed with random hexamer primers. cDNA fragments were end-repaired using T4 polymerase, T4 PNK and Klenow DNA polymerase and a protruding “A” base was added to the 3′ ends of the DNA fragments for the ligation with Illumina adaptors with “T” overhangs. After adapter ligation, cDNA in the size range of 200+/−25 bp was selected for PCR amplification and sequenced on an Illumina GAII or HighSeq for 1×36 bp (single-end sequencing protocol) according to the manufacturer's instructions.

### Computational Analysis

Spliced Alignment of mRNA-Seq and Protein Occupancy Short Reads

We used tophat (version 1.32) (Trapnell et al., 2009) for spliced alignment of paired-end and single-end reads to the human reference genome sequence (hg18). Prior knowledge on candidate splice junctions was obtained from EnsEMBL (release 54, www.ensembl.org) to increase the sensitivity of the mapping process.

### RNA Preparation and Enrichments Analysis

We computed transcript abundance estimates (FPKM values) using cufflinks (version 1.03; cite) with options—frag-bias-correct and—multi-read-correct. The course of RNA preparation was monitored using pairwise scatter plots of these FPKM values. The read count distribution over different RNA class (mRNA, rRNA and other) was inferred by multiplying the FPKM values with the respective length of the longest transcripts for a given gene.

### Read Coverage Plots for Human RefSeq Transcripts

Annotation files of human RefSeq transcripts were obtained from the Table Browser of the UCSC genome browser (http://genome.ucsc.edu/cgi-bin/hgTables?command=start; release hg18). Bed files for entire transcripts, 5′UTR, 3′UTR and coding regions were retrieved separately. Only records with annotated translation start and stop sites were kept. The BAM file of the merged protein occupancy profiling libraries was used to determine the per base coverage. This per base coverage was normalized by the maximal read coverage of the region of interest. We employed the coverageBed tool (Quinlan and Hall, 2010) to compute profiles for individual exons. These profiles are stitched together and relative positions are computed after normalizing for transcript length by discretizing coordinates into 100 bins for each transcript.

### Computing Conservation Scores of Sites in 3′ UTRs

We collected all T-to-C conversion sites with at least 2 conversion events from RefSeq 3′UTR regions. We centered a 3 nt window around each site and computed the average phylogenetic conservation within this window. We used the PhyloP (cite) score to measure sequence conservation. The corresponding file retrieved from the UCSC site (phyloP44wayPlacMammal wiggle track). For our background model, we collected all T positions within TUTRs, which had zero conversion events. Average conservation scores were then computed in the same way.

### Genome-Wide Base Coverage and T-to-C Conversion Profiles

Protein occupancy profiling short reads were generated with a strand-specific protocol. We separated all reads by strand and generated two strand-specific mpileup file with samtools 0.1.18 (Li et al., 2009). These file were subsequently input into custom PERL scripts to produce a separate bedgraph file for each strand (Watson/Crick). Bedgraph files were loaded into our local UCSC hg18 genome browser instance for visualization purposes. Additionally, a single bedgraph file for strand-specific T-to-C conversions was produced in a similar manner. T-to-C conversion sites are only included in the final file if at least two conversion events were observed.

### Genome-Wide Statistics of Read Mutation Patterns

We collected all single base mutations events from the BAM file of aligned reads using the calmd command from samtools (Li et al., 2009). Reads were classified by their edit distance (0,1,2) and as unique or multi-mappers. Read mutation spectra were computed from uniquely mapping reads with an edit distance of 1.

### Analysis of Overrepresented Protein Domains

### SCOP Superfamily Enrichment

Potential RNA binding proteins were queried against the Proteome Folding Project database (PFP) (Drew et al., 2011), a database of protein structure and domain boundary predictions spaming>100 complete genomes. This database provided SCOP superfamily classifications derived from sequence similarity (psi-blast), fold recognition and Rosetta de novo structure prediction for proteins for RNA-binding proteins (and their close homologs in other species in the database). SCOP classifications discovered via PDB-Blast, FFAS03, and de novo structure prediction (with a 0.8 confidence threshold) were used for fold enrichment analysis. From these sets of SCOP superfamilies, an enrichment analysis as described in Drew et al. was performed. Additionally, a fisher t-test (R package ‘fisher’) was used to find significantly enriched superfamilies over a background of the full human proteome (Uniprot, July 2011). P-values were Bonferroni corrected based on total number of unique superfamilies found in the set.

To expand our protein fold annotation coverage of the newly discovered human RNA-binding proteins, we conducted a second enrichment analysis that included fold designations derived from close homologs of the human RNA-binding proteins in six organisms: human, mouse, Caenorhabditis elegans, Escherichia coli, Saccharomyces cerevisiae, and Arabidopsis thaliana. To find the best and most representative homolog for each putative human RNA binding protein (RBP) sequence, we blasted the human RBP set against the proteomes of the six organisms, keeping the best 250 results for each sequence. Of the 250 blast results, we saved those with greater than 50% identity over 80% sequence length, a conservative threshold on proteins' sharing the same SCOP superfamilies. Of these filtered results, we then chose the homolog sequence with the best blast score and the highest-probability SCOP superfamily predictions. With the set of SCOP superfamilies obtained from considering the best homologs for each novel human RBP sequence, we conducted the same enrichment analysis as described above. In all cases fold enrichments are separately reported for each quantification group (proteins seen in 3, 2, and 1 replicate experiment).

### Pfam and InterPro Domain Enrichment

We carried out a similar enrichment analysis as the SCOP enrichment to determine Pfam functional families and InterPro signatures (IPRs) that are overrepresented in each of the quantification sets. We first compiled a data set of all human protein sequences from Uniprot stripped of 90% identical sequences to reduce computation time and redundancy. To this set we added the 773 novel human RNA proteins from this experiment. We then ran InterPro first with only Pfam enabled, and then with all sources enabled. The Pfam families and InterPro signatures found in the novel human RNA-binding sequences formed the enrichment sets for Pfam and InterPro enrichment analysis, respectively, while the family and signature hits for the non-redundant human protein sequences as a whole formed the background sets for each analysis. Again, we split the RNA-binding proteins by quantification group, compiled Pfam family and InterPro signature sets for all groups, and ran enrichment analysis (as described for SCOP folds above) against the background of Pfam and InterPro results for our set of non-redundant human protein sequences.

### Function Prediction

Predictions for the GO Molecular Function term RNA binding, along with first-generation child terms of RNA binding, were calculated using our implementation of the GeneMANIA algorithm of (Mostafavi et al., 2008) modified as described below. The GeneMANIA algorithm was chosen because of its strong performance in the MouseFunc function prediction competition (Pena-Castillo et al., 2008), and its computational tractability which allowed us to quickly run predictions on our large set of 49,518 non-redundant human sequences. Briefly, the GeneMANIA algorithm is a form of Gaussian-field label propagation that operates on a functional association network whose edges define the affinity between genes given a functional context, generated as a weighted combination of a number of association networks. For this work we combined several network types to make function predictions including: i) a network of GO-process and localization similarities, ii-iv) similarities in InterPro and Pfam domain content, v) protein-protein interactions, vi) co-expression relationships, and vii) structural similarity derived from the Proteome Folding database (Drew et al., 2011). Each node of the graph is a gene which may be previously known to have the function in question, known to not possess that function, or may be unlabeled (here we focus on RNA-binding, its child GO-functions, and DNA-binding). The network edges are generated by an optimization step that maximizes the functional similarity inherent in a set of heterogeneous data-types in the presence of the known labels, (the weights on the influence of each network type are learned from a training set of already annotated proteins separately for each function label we try to predict, as described in (Mostafavi et al., 2008)). Once labels have been propagated on this composite network, discriminant thresholds are chosen to assign predictions to unlabeled sequences.

### Data Sets Used for Function Prediction and Network Figure Generation

Our version of the GeneMANIA function prediction algorithm makes use of three categorical data types (InterPro family, Pfam family, and GO Biological Process and Cellular Component annotation), a protein-protein interaction network, a co-expression network, and a structure-similarity network for a total of 6 raw data-types. Only the top 100 similarity scores were kept for each sequence and in each data-type, in order to keep the networks sparse, but in the case of PPI data, the sparsity was much greater as the average number of interactions for a sequence that had any know interactions was only 18.

### Categorical Data

For each categorical data-type, we create a binary feature vector whose length is the total number of unique categories that appeared in any of the sequences. As in (Mostafavi et al., 2008), we transform this binary vector by turning all 1's into −log(B), and all 0's into log(1-B), where B is the proportion of sequences that have the given feature, thus allowing rarer features to contribute more in the similarity calculation. The network is then constructed from this transformed feature matrix by taking the pairwise Pearson Correlation Coefficients. InterPro and Pfam results were obtained by querying the 49,518 non-redundant sequences against the InterPro database, Release 34.0, (Hunter et al., 2011). Go annotations were obtained from querying the known GI numbers of the sequences against the AgBase Go Retriever (McCarthy et al., 2006).

### Gene Expression Data

Gene expression data was obtained from two assays: HG-U133_Plus_{—}2, and U133AAofAv2, which combined have a total of 368 cell types/conditions. The data for each assay was normalized individually using the Affy library in R, and the resulting two expression vectors for each gene were concatenated into one vector. Since expression data is collected at the gene-level, we had to map our sequences to gene names that appeared in the two assays. The network was then created as the pairwise PCC of expression vectors.

### Protein-Protein Interaction Data:

Protein-protein interaction data was collected from the BioNetBuilder project (Avila-Campillo et al., 2007). The network was left as a binary network, with a 1 if two proteins interacted and a 0 otherwise.

### Structure Similarity Data:

The set of 49,519 non-redundant human protein sequences, including novel RNA binding protein sequences, was blasted against a database of proteins previously annotated with astral structural coverage (Brenner et al., 2000). This database consisted of the proteomes of six organisms: human, mouse, Caenorhabditis elegans, Escherichia coli, Saccharomyces cerevisiae, and Arabidopsis thaliana. For each sequence in the non-redundant human set, the best 250 blast results were filtered to retain sequences with at least 50% identity over 80% sequence length. From these filtered blast results, a best homolog with astral structural coverage was chosen to represent the source human sequence, where the best homolog was considered to be the best blast match with the best structural coverage. Structural coverage was computed by considering all domains of a homolog protein. Each domain is either covered wholly or partially by an astral structure, or not annotated with structure. Structural coverage is the average over the number of domains of the proportion of each domain that is covered by astral structure (for domains without astral structure annotation, proportion covered is 0). Domains are annotated with astral structures by matching the regions of domains assigned to PDB structures via the Ginzu pipeline (Drew et al., 2011) to the regions of those PDB structures covered by astral structures. Each domain-to-astral structure annotation was scored with a percentage of sequence-space overlap.

For the best blast result with the highest structural coverage, the astral structures matching each domain of the protein were stored. If a domain was not annotated with astral structure, a null placeholder was included in this list of astrals to represent a domain without structural coverage. In this way, each source human protein is represented by a list of covering astral structures that can be considered in a protein-vs-protein comparison based on structural similarity. From conservative choosing of homolog proteins based on sequence identity and high structural coverage, we were left with roughly 23,000 proteins to compare. Prior to this analysis, we computed the structural similarity of all astral structures to each other by MCM (mammoth confidence metric) score, described in (Drew et al., 2011). With these pre-computed structure similarities, we calculated the all-vs.-all homolog protein structure matrix for these 23,000 proteins, keeping only the 100 most structurally similar proteins for each source protein.

Structural similarity between two proteins was computed as the sum of the maximum pairwise score between each structure representing each protein averaged over the total number of domains in both proteins. If the similarity score of a source and target protein was in the best 100 scores for that source protein, the score for the pair was added to the structure all-vs.-all matrix. This effort was extremely computationally demanding (23,000 by 23,000 sets of operations), and so was split into 500 parts and run on a compute cluster.

### Association Network Combination:

For the network combination algorithm of (Mostafavi et al., 2008), we chose the unregularized version as the regularization described in the paper seemed to dominate the function-specific contributions of each data-set. The unregularized version solves the optimization problem:

α=argmin_{α′}(Ωα′−t)^{t}(Ωα′−t)

Where α is the set of optimal weights, and Ω and t are the positive-positive positive-negative pair weight matrix and the target vector described in (Mostafavi et al., 2008).

Positive examples were chosen as any sequence annotated as having the function in question, or with any child of the function in question, and negative examples were sequences with GO molecular function annotations that were not the function in question or a child of the function in question. Additionally, each network, and the final composite network, was normalized as in (Mostafavi et al., 2008).

Unlike in GeneMANIA where each node in the network was a gene, in our network nodes represent sequences, and as some data-types contain information at the sequence level, and others at the gene level, the coverage of each data-type is not consistent. Additionally some data-types are simply more comprehensive, such as InterPro, which returned results for 38,396 sequences, compared to Pfam, which only returned hits for 35,082 (Table X.X shows the coverage of each data-type). Because the objective function rewards low similarity for negative example pairs, a data-type with less coverage and therefore more sparsity will get an unfair weight boost in the final network. To remedy this problem, we only construct Omega from pairs of omni-reachable sequences, where a sequence is defined as omni-reachable if it is in a row that contains at least one non-zero entry from each data-type. If a data-type is dropped by the algorithm due to a negative weight assignment, the set of omni-reachable sequences is re-calculated given the remaining subset of data types (and can only grow larger by doing so).

### Label Propagation Function Prediction and Cross Validation:

Once the combined network is calculated, discriminant values are calculated as the solution to:

$${f}^{*} = {argmin}_{f} {\sum }_{i} {( {f}_{i} - {y}_{i} )}^{2} + {\sum }_{i} {\sum }_{j} {{w}_{ij} ( {f}_{i} - {f}_{j} )}^{2}$$

Where the w's are the weights from the combined network and y is a bias vector representing your prior knowledge about positive and negative examples, and your prior belief about unlabeled sequences, as in (Mostafavi et al., 2008).

Threshold values for the discriminant were obtained through k-fold cross-validation. For each of the k calculations, the known labels are dropped on a random leave-out set of size 49,518/k, which contains the same proportion of positive, negative, and unlabeled sequences as the entire set. The discriminant threshold is then varied until the desired precision level is met on the leave-out set, and the recall value for the discriminant threshold is noted. If the desired precision level is unattainable for any discriminant threshold value, then that particular cross-validation run is not counted in the final totals.

Once cross validation is complete, the discriminant threshold value for a given precision is calculated as the average of values for all of the cross-validation tests. We chose to predict functions at precision levels of 80%, 50% and 20%, and set k=10 for the functions of RNA binding and DNA binding, but k=5 for the children of RNA binding to allow for enough positive labels in each of the leave-out sets. Table XN.1 shows the recall values at each precision for the different function labels.

### Function Prediction Benchmarking

We selected the function prediction algorithm used in this work based on the mouseFunc evaluation of function prediction methods (Pena-Castillo et al., 2008), and accordingly, we used the mouseFunc performance measures to benchmark our modified implementation of the core geneMania algorithm (where major modifications include those described above, the use of additional protein 3D structural features, and the growth of the data-sets used in the last 1.5 years). MouseFunc evaluated algorithms by using several measures: precision rates at fixed recalls of 1%, 10%, 20%, 50%, 80% and 100%, the AUC_{—}50 measure (area under the ROC curve up to the first 50 false positives), and the recall at a false positive rate of 1%. GO function categories were divided by the number of genes associated with a given function, with count ranges of [3-10], [11-30], [31-100], and [101-300] (functions with 3-10 genes in the human genome would be considered “specific” while functions assigned to more than 100 genes would be more general functional labels like “protein kinase binding”, and “RNA binding” would be more general still). Method comparisons were carried out on both a random test set of mouse genes, as well as a set of genes for which novel functional annotations were deposited after the training set of function labels and raw data used for prediction was collected (the second set of proteins thus serves as a reasonable proxy for true blind predictions). Table XN.2 shows the performance of our algorithm (marked humMania) on RNA binding child terms, averaged over different levels of functional specificity. We exclude from consideration function labels with fewer than 10 gene products in the human genome, as our focus is on a general functional term “RNA binding” here, but provide statistics obtained from RNA binding children in the other specificity levels used in mouseFunc: [11-30], [31-100], and [101-300]). We compared our modified algorithm to the performance of GeneMania and the other leading MouseFunc performer, an ensemble SVM classifier {Guan, 2008 #900}.

HumMania shows strong performance across all specificity levels, often outperforming the methods of Guan and Mostafavi. Of course, this is not a fair comparison, as predictions were done on different organisms, with different base data sets, at different points in time, and for humMania only on a subset of RNA binding-related terms. The goal of this benchmarking, however, is not to demonstrate the superiority of our algorithm over another, but rather to illustrate that our algorithm performs comparably to the current state of the art. The performance of our algorithm and other state of the art algorithms suggests that the RNA-binders that we could not predict are unlikely to be accurately discovered or predicted by any prediction algorithm, and thus represent new RNA-binders (RNA-binders that have novel interactions, structures, domains, and sequence families). To reinforce confidence in our RNA-binder predictions, table XN.3 shows the performance of humMania on the RNA-binding term itself, compared to the performance of Guan and Mostafavi in mouseFunc on that same GO function term. HumMania outperforms these methods in terms of precision at all but the lowest and highest recall values, and exhibits the top AUC score and recall at 1% false positive rate.

It is worth noting that the count of annotated RNA Binders is much higher in our data compared to the count in mouseFunc (1214 in our data, and a specificity range of [101-300] in mouseFunc), which might contribute to the enhanced performance of our algorithm. This is due to the fact that there are more known RNA Binders in human than mouse and that our data is several years newer. We also chose to include IEA annotations when assigning GO labels. This practice is usually avoided due to the lack of curation of IEA annotations and the potential for error propagation. Yet our goal here once again was to provide the most comprehensive set of predictions possible, so that given the demonstrated strength of our predictive algorithm, and our broad threshold for labeling RNA-binders, one can be confident that any RNA-binders that were not predicted even at the 20% precision level, are truly novel. Thus, while typically one wishes to avoid false positives in biology, we, for the purposes of this work, needed to avoid false negatives, and thus included IEA annotations.

### Generation of RNA-Binder Association Network

Networks used for function prediction were output in SIF format, prior to combining networks for function prediction. For each RNA-binder the top 100 (or fewer) network edges for each network type were loaded into Cytoscape (Cline et al., 2007). Previous RNA-binding function annotations and the number of times each RNA-binder was seen (in 1, 2 or 3 experiments) were loaded as node attributes. The network used to generate all network diagrams is available as raw network formats (.sif, .eda and .noa) and Cytoscape files (.cys) as supplemental files. Several Cytoscape plugins (Avila-Campillo et al., 2007; Cline et al., 2007; Konieczka et al., 2009; Shannon et al., 2006; Wozniak et al., 2010) were used for network clustering (APCluster, MINE), communication with other tools (CyGoose (Avila-Campillo et al., 2007; Konieczka et al., 2009), and retrieving protein interactions.

A protein-protein-interaction network was generated for the RNA-binders identified in this study using Cytoscape to analyze the connectivity between them. Protein-Protein interaction data was gathered from the iRefIndex database consolidation, via the iRefScape CytoScape plugin (Razick, 2011 #810). Data was obtained for the list of RNA-binders (examining only intra-list interactions), as well as for a background network to use as a control. This background network consisted of to a theoretical set of expressed proteins deduced from mRNA sequencing data which make up approximately 95% of the total cellular mRNA molecules.

The transcripts were mapped to unique gene symbols, with any of the RNA-binding list members that did not appear in the background added to it manually, creating a final HEK293 Interactome of ˜6400 genes. In order to generate control statistics for comparison with the RNA-binder connectivity, 50 random subsets of this background network were chosen, each the same size as the RNA-binder list, and their clustering coefficients, average degrees, and characteristic path lengths averaged.

### Gene Ontology Terms Enrichment Analysis and Protein Cluster Visualization

We searched for overrepresented GO terms for biological processes in the set of 801 proteins identified by our assay and their reported protein interaction partners (first neighbours in the PPI-network created in cytoscape). Proteins associated with overrepresented GO terms were clustered using the functional annotation tool DAVID (Huang da, 2009 #866), and the protein cluster members and their interactions were extracted from the cytoscape network and presented as sub-networks with the node attributes described above.

### Tables

TABLE 1A
Selected enriched Pfam and Interpro domains

| | | | | Corrected | Enrichment |
|---|---|---|---|---|---|
| Domain | Representative | Pfam | InterPro | P-value | Score |
| RNA-binding domains | | | | | |
| RRM | PABPC1 | PF00076 | IPR000504 | 3.25e−198 | 2.6515 |
| KH (type I and II) | HNRNPK | PF00013 | IPR004087 | 1.51e−63 | 2.6984 |
| dsRNA | STAU1 | PF00035 | IPR014720 | 9.12e−16 | 2.4462 |
| ZnF-CCCH | U2AF1 | PF00642 | IPR000571 | 8.84e−19 | 2.4326 |
| ZnF-CCHC | LIN28B | PF00098 | IPR001878 | 3.22e−17 | 2.5797 |
| S1 | DHX8 | PF00575 | IPR022967 | 1.33e−08 | 2.9551 |
| OB_NTP_bind | DHX9 | PF07717 | IPR012340 | 8.01e−09 | 2.8186 |
| Pumilio | Pum1 | PF00806 | IPR001313 | 2.75e−09 | 2.2702 |
| LSM | LSM14A | PF01423 | IPR006649 | 4.22e−05 | 2.6680 |
| MIF4G | EIF4G1 | PF02854 | IPR016021 | 0.0451 | 2.1861 |
| SAP | HNRNPU | PF02037 | IPR003034 | 8.05e−08 | 2.4888 |
| YTH | YTH | PF04146 | IPR007275 | 5.59e−05 | 3.5842 |
| ColdShock | LIN28B | PF00313 | IPR011129 | 6.54e−05 | 2.5353 |
| PurA | PURA | PF04845 | IPR006628 | 0.0234 | 3.5003 |
| PPR | LRPPRC | PF01535 | IPR002885 | 1.12e−06 | 2.9771 |
| PWI | SRRM1 | PF01480 | IPR002483 | 0.0012 | 3.1323 |
| La | SSB | PF05383 | IPR006630 | 0.0047 | 2.4443 |
| Putative | | | | | |
| RNA-binding domains | | | | | |
| DUF1220 | NBPF10 | PF06758 | IPR010630 | 5.22e−23 | 2.1837 |
| zf-NF-X1 | NFX1 | PF01422 | IPR000967 | 0.0049 | 2.7584 |
| SWAP/SURP | SF3A1 | PF01805 | IPR000061 | 0.0002 | 2.6429 |
| HMG box | HMGB1 | PF00505 | IPR000910 | 0.0027 | 1.5167 |
| DZF | ILF3 | PF07528 | IPR006561 | 0.0086 | 3.0949 |
| DUF1897 | KHSRP | PF09005 | IPR015096 | 0.0152 | 2.9771 |
| HAT helix | SART3 | PF02184 | IPR003107 | 0.0101 | 2.7576 |
| RAP | FASTKD1 | PF08373 | IPR013584 | 0.0565 | 2.6894 |

TABLE 1B
Selected enriched SCOP superfamily folds

| | | | Corrected | Enrichment |
|---|---|---|---|---|
| Domain | Representative | SCOP | P-value | Score |
| RNA-binding | | | | |
| domains | | | | |
| RBD | PABPC1 | d.58.7 | 1.04e−120 | 2.6238 |
| KH (type I) | HNRNPK | d.51.1 | 7.63e−20 | 2.5681 |
| dsRNA | STAU1 | d.50.1 | 2.11e−08 | 2.3766 |
| PAZ | EIF2C1 | b.34.14 | 1.1586 | 2.6150 |
| LSM | LSM14A | b.38.1 | 7.84e−06 | 2.6468 |
| PWI | SRRM1 | a.188.1 | 0.8794 | 2.7486 |
| Putative | | | | |
| RNA-binding | | | | |
| domains | | | | |
| HMG box | HMGB1 | a.21.1 | 1.62e−11 | 2.0971 |
| “Winged helix” | DDX54 | a.4.5 | 1.33e−06 | 1.2081 |
| DNA-binding | | | | |
| AlbA-like | C9orf23 | d.68.6 | 1.50e−06 | 3.5959 |

TABLE 2
GO term overrepresenation

| GO ID | Term | Count | % | p-value |
|---|---|---|---|---|
| GO: 0008380 | RNA splicing | 163 | 7.4 | 8.2E−64 |
| GO: 0006397 | mRNA processing | 170 | 7.8 | 1.2E−59 |
| GO: 0006412 | translation | 142 | 6.5 | 2.8E−36 |
| GO: 0006974 | response to DNA damage | 122 | 5.6 | 3.6E−36 |
| GO: 0006351 | transcription, DNA-dependent | 101 | 4.6 | 7.4E−18 |
| GO: 0050658 | RNA transport | 44 | 2.0 | 1.3E−12 |
| GO: 0032508 | DNA duplex unwinding | 12 | 0.5 | 7.0E−06 |

TABLE S2
mRNA-bound proteins identified by quantitative mass spectrometry
(enrichment of at least 3-fold in at least one of three analyses)

| | | | | |
|---|---|---|---|---|
| NP_003783 | NP_036339 | NP_004957 | NP_004629 | NP_006684 |
| NP_892006 | NP_073750 | NP_005511 | NP_116184 | NP_002678 |
| NP_000933 | NP_919223 | NP_066964 | NP_733829 | NP_057121 |
| NP_110379 | NP_001347 | NP_004631 | NP_056393 | NP_005372 |
| NP_006537 | NP_001108206 | NP_008971 | NP_001073027 | NP_058520 |
| NP_006538 | NP_002128 | NP_115687 | NP_002015 | NP_892021 |
| NP_006363 | NP_067000 | NP_005745 | NP_055970 | NP_004634 |
| NP_002559 | NP_001136402 | NP_597709 | NP_009210 | NP_001271 |
| NP_006588 | NP_001129125 | NP_060518 | NP_008835 | NP_006749 |
| NP_009011 | NP_001408 | NP_112740 | NP_005849 | NP_055288 |
| NP_055205 | NP_067013 | NP_005841 | NP_079087 | NP_055186 |
| NP_002130 | NP_003893 | NP_001231827 | NP_006549 | NP_004621 |
| NP_055278 | NP_001229820 | NP_002958 | NP_055554 | NP_001092104 |
| NP_006796 | NP_006297 | NP_003008 | NP_443111 | NP_005096 |
| NP_001013653 | NP_006793 | NP_005327 | NP_056474 | NP_004891 |
| NP_573566 | NP_031401 | NP_002810 | NP_003767 | NP_001316 |
| NP_001524 | NP_066014 | NP_055662 | NP_036429 | NP_003125 |
| NP_005057 | NP_001120664 | NP_001009 | NP_002361 | NP_003744 |
| NP_003925 | NP_055427 | NP_001460 | NP_056050 | NP_057710 |
| NP_001095868 | NP_006550 | NP_066368 | NP_005821 | NP_510965 |
| NP_002131 | NP_062543 | NP_004740 | NP_057191 | NP_005990 |
| NP_001410 | NP_005773 | NP_060564 | NP_085130 | NP_004388 |
| NP_001348 | NP_006734 | NP_001073888 | NP_653307 | NP_001129107 |
| NP_112738 | NP_004550 | NP_056176 | NP_003760 | NP_001136113 |
| NP_112556 | NP_005959 | NP_055555 | NP_054797 | NP_073605 |
| NP_006377 | NP_005917 | NP_071505 | NP_079539 | NP_001028260 |
| NP_116147 | NP_001000 | NP_001026854 | NP_699198 | NP_060616 |
| NP_057951 | NP_114032 | NP_006187 | NP_057216 | NP_003479 |
| NP_003243 | NP_056422 | NP_001035879 | NP_112487 | NP_065823 |
| NP_001018494 | NP_006539 | NP_005782 | NP_005078 | NP_149080 |
| NP_001181884 | NP_003743 | NP_003746 | NP_005007 | NP_056132 |
| NP_001008661 | NP_001070910 | NP_742068 | NP_006319 | NP_001107590 |
| NP_003676 | NP_003642 | NP_079215 | NP_689929 | NP_060268 |
| NP_055277 | NP_071496 | NP_053733 | NP_001171890 | NP_060517 |
| NP_004951 | NP_112420 | NP_009176 | NP_060422 | NP_060548 |
| NP_001022 | NP_057154 | NP_002083 | NP_003134 | NP_473357 |
| NP_005850 | NP_001005 | NP_066997 | NP_055987 | NP_068593 |
| NP_001018077 | NP_036565 | NP_620412 | NP_004587 | NP_001020767 |
| NP_003741 | NP_510880 | NP_008855 | NP_005795 | NP_653304 |
| NP_150093 | NP_055464 | NP_001020 | NP_066363 | NP_002433 |
| NP_060090 | NP_059965 | NP_004851 | NP_057131 | NP_775738 |
| NP_004506 | NP_004387 | NP_057034 | NP_006266 | NP_005110 |
| NP_001138880 | NP_001407 | NP_055485 | NP_008938 | NP_056299 |
| NP_631961 | NP_000996 | NP_004584 | NP_115551 | NP_036453 |
| NP_002902 | NP_055309 | NP_004719 | NP_054737 | NP_060060 |
| NP_055642 | NP_775930 | NP_065757 | NP_005443 | NP_006744 |
| NP_001181875 | NP_055829 | NP_757386 | NP_061856 | NP_037450 |
| NP_031385 | NP_055727 | NP_054722 | NP_060289 | NP_001317 |
| NP_624311 | NP_006833 | NP_005828 | NP_001036100 | NP_277028 |
| NP_001032405 | NP_005792 | NP_003751 | NP_000929 | NP_065916 |
| NP_001170853 | NP_006353 | NP_056418 | NP_060791 | NP_060681 |
| NP_036387 | NP_008856 | NP_005889 | NP_115714 | NP_110438 |
| NP_775882 | NP_065101 | NP_060014 | NP_001349 | NP_612395 |
| NP_115866 | NP_060850 | NP_004932 | NP_056311 | NP_001093392 |
| NP_031368 | NP_036552 | NP_036286 | NP_113680 | NP_057475 |
| NP_061862 | NP_009096 | NP_002705 | NP_006702 | NP_060365 |
| NP_057040 | NP_005608 | NP_057055 | NP_001073884 | NP_149103 |
| NP_694453 | NP_056494 | NP_000974 | NP_002506 | NP_055181 |
| NP_008937 | NP_037425 | NP_005769 | NP_036340 | NP_067062 |
| NP_064621 | NP_001004317 | NP_054879 | NP_036270 | NP_003160 |
| NP_031388 | NP_060857 | NP_036377 | NP_057280 | NP_060547 |
| NP_114108 | NP_060755 | NP_060853 | NP_940888 | NP_009139 |
| NP_056130 | NP_001096617 | NP_003681 | NP_057175 | NP_060228 |
| NP_056648 | NP_115664 | NP_057085 | NP_079100 | NP_079170 |
| NP_055744 | NP_001157852 | NP_037507 | NP_055994 | NP_000998 |
| NP_003007 | NP_001026865 | NP_002374 | NP_078947 | NP_065118 |
| NP_055983 | NP_073739 | NP_005617 | NP_057396 | NP_055816 |
| NP_002887 | NP_001403 | NP_612403 | NP_055792 | NP_056473 |
| NP_001020248 | NP_001191397 | NP_055118 | NP_057134 | NP_004238 |
| NP_056992 | NP_542781 | NP_976324 | NP_057474 | NP_060498 |
| NP_872578 | NP_055506 | NP_002943 | NP_066953 | NP_057368 |
| NP_660341 | NP_149073 | NP_055699 | NP_068598 | NP_055871 |
| NP_001427 | NP_001087194 | NP_056235 | NP_003119 | NP_066018 |
| NP_004423 | NP_001958 | NP_619520 | NP_054872 | NP_004710 |
| NP_056290 | NP_001152849 | NP_006819 | NP_003161 | NP_036331 |
| NP_004930 | NP_115700 | NP_001139373 | NP_115727 | NP_006378 |
| NP_909122 | NP_002889 | NP_001153408 | NP_055640 | NP_777573 |
| NP_588611 | NP_001102 | NP_001010867 | NP_612453 | NP_005137 |
| NP_113608 | NP_055121 | NP_006766 | NP_079031 | NP_001885 |
| NP_060318 | NP_062535 | NP_945314 | NP_036524 | NP_037489 |
| NP_002507 | NP_005751 | NP_001244 | NP_003578 | NP_079491 |
| NP_076950 | NP_005861 | NP_065801 | NP_054733 | NP_060362 |
| NP_057174 | NP_112223 | NP_064504 | NP_037418 | NP_004732 |
| NP_078898 | NP_001091977 | NP_006161 | NP_056139 | NP_002286 |
| NP_001099008 | NP_002964 | NP_060858 | NP_112179 | NP_064615 |
| NP_060217 | NP_055692 | NP_001409 | NP_112483 | NP_115766 |
| NP_057342 | NP_113640 | NP_006697 | NP_115725 | NP_003128 |
| NP_005768 | NP_004085 | NP_031398 | NP_055312 | NP_006640 |
| NP_689971 | NP_005868 | NP_115622 | NP_001139542 | NP_060225 |
| NP_976049 | NP_659002 | NP_005866 | NP_057267 | NP_002261 |
| NP_005144 | NP_006038 | NP_054894 | NP_054899 | NP_008941 |
| NP_006322 | NP_444271 | NP_001315 | NP_057508 | NP_113673 |
| NP_056360 | NP_001035526 | NP_006436 | NP_061874 | NP_055323 |
| NP_036473 | NP_076971 | NP_001011 | NP_115487 | NP_001135757 |
| NP_006764 | NP_680544 | NP_114403 | NP_001139699 | NP_002939 |
| NP_003674 | NP_002262 | NP_055907 | NP_002366 | NP_003694 |
| NP_001096123 | NP_001177779 | NP_001012 | NP_000964 | NP_001632 |
| NP_620305 | NP_003709 | NP_008841 | NP_001124439 | NP_000928 |
| NP_078893 | NP_000250 | NP_001148 | NP_055706 | NP_078804 |
| NP_057439 | NP_067054 | NP_005013 | NP_073568 | NP_003162 |
| NP_004452 | NP_004841 | NP_056444 | NP_057588 | NP_872634 |
| NP_004389 | NP_005717 | NP_001155091 | NP_055701 | NP_000967 |
| NP_004999 | NP_689813 | NP_002511 | NP_002945 | NP_001010 |
| NP_057103 | NP_079128 | NP_003133 | NP_001609 | NP_006214 |
| NP_542199 | NP_057737 | NP_689592 | NP_078938 | NP_001021 |
| NP_001559 | NP_115285 | NP_037374 | NP_000962 | NP_000980 |
| NP_057589 | NP_061744 | NP_060485 | NP_060542 | NP_001032726 |
| NP_055984 | NP_849152 | NP_075066 | NP_000958 | NP_057417 |
| NP_001020262 | NP_005989 | NP_005040 | NP_000997 | NP_004809 |
| NP_008869 | NP_001527 | NP_005026 | NP_000981 | NP_000993 |
| NP_057185 | NP_071349 | NP_919307 | NP_006089 | NP_002087 |
| NP_001128715 | NP_976043 | NP_057733 | NP_002701 | NP_075388 |
| NP_976225 | NP_061825 | NP_055516 | NP_003926 | NP_056269 |
| NP_149105 | NP_001136113 | NP_065147 | NP_110425 | NP_000979 |
| NP_689759 | NP_008911 | NP_005753 | NP_006383 | NP_001019 |
| NP_061870 | NP_001002909 | NP_000966 | NP_001013 | NP_060441 |
| NP_004484 | NP_004441 | NP_071353 | NP_001009881 | NP_000959 |
| NP_001058 | NP_115735 | NP_001104792 | NP_056306 | NP_009035 |
| NP_057572 | NP_001007 | NP_001008 | NP_001002 | NP_072045 |
| NP_758455 | NP_004528 | NP_004068 | NP_036411 | NP_001393 |
| NP_055568 | NP_065761 | NP_061185 | NP_061164 | NP_002119 |
| NP_055496 | NP_006603 | NP_055412 | NP_000976 | NP_003339 |
| NP_003080 | NP_112598 | NP_006316 | NP_000977 | NP_690002 |
| NP_000987 | NP_064528 | NP_077295 | NP_005336 | NP_005310 |
| NP_008868 | NP_001157973 | NP_077289 | NP_073616 | NP_003463 |
| NP_060117 | NP_001001998 | NP_848927 | NP_001032412 | NP_006704 |
| NP_005830 | NP_005909 | NP_071761 | NP_009123 | NP_002565 |
| NP_071896 | NP_057284 | NP_008924 | NP_061915 | NP_002120 |
| NP_003137 | NP_001137232 | NP_734467 | NP_003192 | NP_778236 |
| NP_055644 | NP_060418 | NP_056525 | NP_001003 | NP_031381 |
| NP_004759 | NP_001262 | NP_004623 | NP_057736 | NP_000989 |
| NP_005312 | NP_060316 | NP_003325 | NP_000975 | NP_000973 |
| NP_004578 | NP_009057 | NP_004025 | NP_003370 | NP_852615 |
| NP_115883 | NP_004865 | NP_055318 | NP_003575 | NP_008957 |
| NP_653205 | NP_115544 | NP_000978 | NP_060579 | NP_002495 |
| NP_003081 | NP_000971 | NP_006827 | NP_057018 | NP_001127705 |
| NP_071401 | NP_009109 | NP_001016 | NP_002287 | NP_060597 |
| NP_037367 | NP_079030 | NP_001129123 | NP_003277 | NP_001124151 |
| NP_036255 | NP_002219 | NP_001952 | NP_055693 | NP_057306 |
| NP_073754 | NP_076991 | NP_003084 | NP_003964 | NP_954981 |
| NP_835461 | NP_683759 | NP_005079 | NP_001518 | NP_001123500 |
| NP_056277 | NP_002256 | NP_004690 | NP_149098 | NP_000963 |
| NP_000909 | NP_659419 | NP_001035374 | NP_008974 | NP_001070667 |
| NP_062552 | NP_004850 | NP_683685 | NP_612409 | NP_001988 |
| NP_115721 | NP_079341 | NP_079140 | NP_003899 | NP_006004 |
| NP_055885 | NP_001485 | NP_059998 | NP_005311 | NP_005337 |
| NP_008878 | NP_066930 | NP_055521 | NP_001017963 | NP_115570 |
| NP_004095 | NP_003320 | NP_037417 | NP_066289 | NP_001737 |
| NP_478126 | NP_006296 | NP_003553 | NP_006017 | NP_003904 |
| NP_002464 | NP_078959 | NP_004689 | NP_150091 | NP_002071 |
| NP_001034792 | NP_061903 | NP_000025 | NP_110390 | NP_937859 |
| NP_149072 | NP_004399 | NP_000983 | NP_001367 | NP_001029249 |
| NP_001019398 | NP_001182061 | NP_149100 | NP_005309 | NP_004166 |
| NP_004837 | NP_976033 | NP_004598 | NP_005508 | NP_077003 |
| NP_116253 | NP_064716 | NP_001960 | NP_066357 | NP_004783 |
| NP_001157789 | NP_060941 | NP_066553 | NP_060707 | NP_000960 |
| NP_056350 | NP_057004 | NP_055048 | NP_001104026 | NP_006816 |
| NP_002257 | NP_004125 | NP_056988 | NP_036457 | NP_060502 |
| NP_071383 | NP_002085 | NP_036222 | NP_055791 | NP_005333 |
| NP_115710 | NP_005693 | NP_002477 | | |
| NP_001094058 | NP_060286 | NP_004695 | | |

TABLE S2
sub-group (267 proteins, which have not been previously annotated as RNA-binding)

| | | | | |
|---|---|---|---|---|
| NP_003783 | NP_001073888 | NP_001129107 | NP_036552 | NP_001010867 |
| NP_892006 | NP_056176 | NP_001028260 | NP_001096617 | NP_006697 |
| NP_000933 | NP_005782 | NP_149080 | NP_115700 | NP_005443 |
| NP_110379 | NP_066997 | NP_001107590 | NP_055121 | NP_001036100 |
| NP_006588 | NP_055485 | NP_005110 | NP_062535 | NP_000929 |
| NP_116147 | NP_004629 | NP_056299 | NP_005751 | NP_060791 |
| NP_003243 | NP_008835 | NP_060060 | NP_005861 | NP_115714 |
| NP_001136402 | NP_005849 | NP_055642 | NP_112223 | NP_056311 |
| NP_006297 | NP_055554 | NP_775882 | NP_001091977 | NP_036340 |
| NP_006793 | NP_699198 | NP_055744 | NP_113640 | NP_036270 |
| NP_005917 | NP_001171890 | NP_056290 | NP_001035526 | NP_055994 |
| NP_056422 | NP_060422 | NP_060318 | NP_056418 | NP_078947 |
| NP_036565 | NP_055987 | NP_078898 | NP_036377 | NP_057396 |
| NP_510880 | NP_066363 | NP_001099008 | NP_060853 | NP_057474 |
| NP_005327 | NP_115551 | NP_005144 | NP_057085 | NP_066953 |
| NP_055662 | NP_002678 | NP_056360 | NP_055118 | NP_068598 |
| NP_004740 | NP_057121 | NP_006833 | NP_056235 | NP_003119 |
| NP_060564 | NP_892021 | NP_065101 | NP_001153408 | NP_055640 |
| NP_612453 | NP_008868 | NP_001148 | NP_009123 | NP_078959 |
| NP_036524 | NP_071896 | NP_005013 | NP_003192 | NP_004399 |
| NP_056139 | NP_003137 | NP_001155091 | NP_003370 | NP_001182061 |
| NP_112483 | NP_004578 | NP_689592 | NP_002287 | NP_976033 |
| NP_115725 | NP_073754 | NP_037374 | NP_003277 | NP_060941 |
| NP_001139542 | NP_835461 | NP_060485 | NP_000928 | NP_002085 |
| NP_037450 | NP_002262 | NP_005040 | NP_078804 | NP_060286 |
| NP_277028 | NP_003709 | NP_005026 | NP_872634 | NP_004690 |
| NP_001093392 | NP_000250 | NP_919307 | NP_006214 | NP_059998 |
| NP_057475 | NP_067054 | NP_057733 | NP_001032726 | NP_037417 |
| NP_149103 | NP_004841 | NP_005753 | NP_075388 | NP_003553 |
| NP_055181 | NP_689813 | NP_071353 | NP_003339 | NP_004598 |
| NP_079170 | NP_057737 | NP_061185 | NP_690002 | NP_055048 |
| NP_056473 | NP_061744 | NP_055412 | NP_003463 | NP_036222 |
| NP_060498 | NP_849152 | NP_006316 | NP_006704 | NP_001518 |
| NP_057368 | NP_005989 | NP_077295 | NP_002120 | NP_149098 |
| NP_055871 | NP_001527 | NP_848927 | NP_008957 | NP_066289 |
| NP_066018 | NP_071349 | NP_008924 | NP_002495 | NP_150091 |
| NP_004710 | NP_976043 | NP_734467 | NP_001127705 | NP_110390 |
| NP_005137 | NP_061825 | NP_056525 | NP_954981 | NP_001367 |
| NP_001885 | NP_001136113 | NP_003325 | NP_001123500 | NP_005508 |
| NP_060362 | NP_008911 | NP_004025 | NP_056277 | NP_060707 |
| NP_064615 | NP_001002909 | NP_055318 | NP_062552 | NP_001104026 |
| NP_003128 | NP_004441 | NP_002366 | NP_002464 | NP_036457 |
| NP_006640 | NP_004528 | NP_060919 | NP_149072 | NP_055791 |
| NP_002261 | NP_006603 | NP_055706 | NP_001019398 | NP_001988 |
| NP_113673 | NP_112598 | NP_073568 | NP_116253 | NP_115570 |
| NP_036473 | NP_004865 | NP_055701 | NP_001157789 | NP_001737 |
| NP_003674 | NP_115544 | NP_001609 | NP_056350 | NP_003904 |
| NP_001096123 | NP_009109 | NP_060542 | NP_002257 | NP_077003 |
| NP_055984 | NP_079030 | NP_006089 | NP_115710 | NP_004783 |
| NP_001128715 | NP_002219 | NP_002701 | NP_002256 | NP_006816 |
| NP_689759 | NP_076991 | NP_003926 | NP_659419 | NP_005333 |
| NP_001058 | NP_683759 | NP_110425 | NP_004850 | |
| NP_057572 | NP_055907 | NP_056306 | NP_001485 | |
| NP_055568 | NP_008841 | NP_061164 | NP_006296 | |

TABLE S4

| | Plasmid used for stable | | | CLIP |
|---|---|---|---|---|
| Protein | transfection | Expression | Immunoprecipitation | assay |
| Controls | | | | |
| CAPRIN1 | pFRT/TO/HIS/FLAG/HA-CAPRIN 1 | positive | positive | positive |
| HNRNPD | pFRT/TO/HIS/FLAG/HA-HNRNPD | positive | positive | positive |
| HNRNPR | pFRT/TO/HIS/FLAG/HA-HNRNPR | positive | positive | positive |
| HNRNPU | pFRT/TO/HIS/FLAG/HA-HNRNPU | positive | positive | positive |
| MYEF2 | pFRT/TO/HIS/FLAG/HA-MYEF2 | positive | positive | positive |
| LDHA | pFRT/TO/FLAG/HA-LDHA | positive | positive | negative |
| PGK1 | pFRT/TO/HIS/FLAG/HA-PGK1 | positive | positive | negative |
| novel mRNA binders | | | | |
| AKAP8L | pFRT/TO/HIS/FLAG/HA-AKAP8L | positive | positive | positive |
| ALKBH5 | pFRT/TO/HIS/FLAG/HA-ALKBH5 | positive | positive | positive |
| API5 | pFRT/TO/HIS/FLAG/HA-API5 | positive | positive | positive |
| BTF3 | pFRT/TO/HIS/FLAG/HA-BTF3 | positive | positive | positive |
| C17orf85 | pFRT/TO/HIS/FLAG/HA-C17orf85 | positive | positive | positive |
| C22orf28 | pFRT/TO/HIS/FLAG/HA-C22orf28 | positive | positive | positive |
| CSNK1E | pFRT/TO/HIS/FLAG/HA-CSNK1E | positive | positive | positive |
| EDF1 | pFRT/TO/HIS/FLAG/HA-EDF1 | positive | positive | positive |
| FAM98A | pFRT/TO/HIS/FLAG/HA-FAM98A | positive | positive | positive |
| IFIT5 | pFRT/TO/HIS/FLAG/HA-IFIT5 | positive | positive | positive |
| KIAA1967 | pFRT/TO/HIS/FLAG/HA-KIAA1967 | positive | positive | positive |
| MKRN2 | pFRT/TO/HIS/FLAG/HA-MKRN2 | positive | positive | positive |
| MYBBP1A | pFRT/TO/HIS/FLAG/HA-MYBBP1A | positive | positive | positive |
| PES1 | pFRT/TO/HIS/FLAG/HA-PES1 | positive | positive | positive |
| PRDX1 | pFRT/TO/HIS/FLAG/HA-PRDX1 | positive | positive | positive |
| SART1 | pFRT/TO/HIS/FLAG/HA-SART1 | positive | positive | positive |
| USP10 | pFRT/TO/FLAG/HA-USP10 | positive | positive | positive |
| YTHDF2 | pFRT/TO/HIS/FLAG/HA-YTHDF2 | positive | positive | positive |
| ZC3H7B | pFRT/TO/HIS/FLAG/HA-ZC3H7B | positive | positive | positive |
| BZW1 | pFRT/TO/HIS/FLAG/HA-BZW1 | positive | positive | negative |
| C16orf80 | pFRT/TO/HIS/FLAG/HA-C16orf80 | positive | positive | negative |
| AKAP1 | pFRT/TO/HIS/FLAG/HA-AKAP1 | positive | negative | |
| CDK13 | pFRT/TO/HIS/FLAG/HA-CDK13 | positive | negative | |
| DUSP11 | pFRT/TO/HIS/FLAG/HA-DUSP11 | positive | negative | |
| MDH2 | pFRT/TO/HIS/FLAG/HA-MDH2 | positive | negative | |
| NKRF | pFRT/TO/FLAG/HA-NKRF | positive | negative | |
| THRAP3 | pFRT/TO/HIS/FLAG/HA-THRAP3 | positive | negative | |
| YARS2 | pFRT/TO/HIS/FLAG/HA-YARS2 | positive | negative | |
| ZC3H18 | pFRT/TO/HIS/FLAG/HA-ZC3H18 | positive | negative | |

TABLE S5
Supplementary Table S5: Summary of PAR-CLIP sequencing data and mRNA targets

| | | | | After | | |
|---|---|---|---|---|---|---|
| | | | Raw | adapter | Unique | Kept unique |
| PAR-CLIP | Seq Run ID | 3′Adapter | reads | removal | sequences | alignments |
| ALKBH5_4SU_1 | ML_MM_48 | NBC8 | 13M | 28% | 23% | 0.15M |
| ALKBH5_4SU_2 | ML_MM_57 | NBC4 | 18M | 27% | 20% | 0.36M |
| C17orf85_4SU_1 | ML_AV_03 | NBC4 | 10M | 39% | 5% | 0.19M |
| C17orf85_4SU_2 | ML_MM_57 | NBC2 | 23M | 56% | 17% | 1.41M |
| C22orf28_4SU_1 | ML_MM_45 | NBC8 | 6M | 34% | 49% | 0.31M |
| C22orf28_4SU_2 | ML_MM_57 | | 17M | 49% | 25% | 1.04M |
| CAPRIN1_4SU_1 | ML_MM_48 | NBC2 | 30M | 87% | 5% | 1.75M |
| CAPRIN1_4SU_2 | ML_YM_05 | NBC2 | 25M | 88% | 32% | 2.33M |
| ZC3H7B_4SU_1 | ML_YM_03 | NBC5 | 8M | 96% | 40% | 1.72M |
| ZC3H7B_4SU_2 | ML_YM_05 | NBC8 | 20M | 88% | 60% | 4.75M |

TABLE S6
Supplementary Table S6: Protein occupancy profling on mRNA sequencing data

| Profiling | | | | After adapter | Unique reads after |
|---|---|---|---|---|---|
| Library | Seq Run ID | 3′Adapter | Raw reads | removal | adapter removal |
| 1 | ML_MM_58 | NBC5 | 61.113.528 | 60.217.076 | 57.887.241 |
| | ML_MM_64 | NBC5 | 35.873.799 | 35.378.270 | 34.209.792 |
| | ML_MM_65 | NBC5 | 37.624.350 | 37.096.683 | 35.861.894 |
| 2 | ML_MM_61 | NBC8 | 40.478.529 | 40.010.280 | 36.372.524 |
| | ML_MM_64 | NBC8 | 38.060.196 | 37.596.817 | 33.867.730 |
| | ML_MM_65 | NBC8 | 39.983.748 | 39.486.152 | 35.541.085 |

TABLE S7

| | Position | dbSNP | dbSNP | dbSNP | db SNP | | | |
|---|---|---|---|---|---|---|---|---|
| | (hg18) | 5′UTR | intron | 3′UTR | intergenic | Gene | Reference | |
| chr.1 | 37731400 | | | rs9253 | | C1orf149 | Genome-wide | BMC Med Genet. |
| | | | | | | | association and | 2007 Sep 19; 8 |
| | | | | | | | linkage analyses of | Suppl 1: S12. |
| | | | | | | | hemostatic factors | |
| | | | | | | | and hematological | |
| | | | | | | | phenotypes in the | |
| | | | | | | | Framingham Heart | |
| | | | | | | | Study. | |
| | 38019740 | | | | rs12117544 | — | Common genetic | Eur J Hum Genet. |
| | | | | | | | variation and | 2010 |
| | | | | | | | performance on | Jul; 18(7): 815-20. |
| | | | | | | | standardized | Epub 2010 Feb 3. |
| | | | | | | | cognitive tests. | |
| | 93076191 | | rs6604026 | | | RPL5 | Genome-wide | Nat Genet. 2009 |
| | | | | | | | association study | Jul; 41(7): 824-8. |
| | | | | | | | identifies new | Epub 2009 Jun 14. |
| | | | | | | | multiple sclerosis | |
| | | | | | | | susceptibility loci on | |
| | | | | | | | chromosomes 12 | |
| | | | | | | | and 20. | |
| | 154135249 | | | rs2282301 | | RIT1 | Does parental | Am J Med Genet B |
| | | | | | | | expressed emotion | Neuropsychiatr |
| | | | | | | | moderate genetic | Genet. 2008 Dec |
| | | | | | | | effects in ADHD? An | 5; 147B(8): 1359-68. |
| | | | | | | | exploration using a | |
| | | | | | | | genome wide | |
| | | | | | | | association scan. | |
| chr.2 | none | | | | | | | |
| chr.3 | none | | | | | | | |
| chr.4 | 20229781 | | | rs1379659 | | SLIT2 | Genome-wide | BMC Med Genet. |
| | | | | | | | association of | 2007 Sep 19; 8 |
| | | | | | | | echocardiographic | Suppl 1: S2. |
| | | | | | | | dimensions, brachial | |
| | | | | | | | artery endothelial | |
| | | | | | | | function and | |
| | | | | | | | treadmill exercise | |
| | | | | | | | responses in the | |
| | | | | | | | Framingham Heart | |
| | | | | | | | Study. | |
| chr.5 | none | | | | | | | |
| chr.6 | 30140501 | | | rs8321 | | ZNRD1 | Genomewide | J Infect Dis. 2009 |
| | | | | | | | association study of | Feb 1; 199(3): 419-26. |
| | | | | | | | an AIDS- | |
| | | | | | | | nonprogression | |
| | | | | | | | cohort emphasizes | |
| | | | | | | | the role played by | |
| | | | | | | | HLA genes (ANRS | |
| | | | | | | | Genomewide | |
| | | | | | | | Association Study | |
| | | | | | | | 02). | |
| chr.7 | 72658273 | | | rs3812316 | | MLXIPL | Genome-wide scan | Nat Genet. 2008 |
| | | | | | | | identifies variation in | Feb; 40(2): 149-51. |
| | | | | | | | MLXIPL associated | Epub 2008 Jan 13. |
| | | | | | | | with plasma | |
| | | | | | | | triglycerides. | |
| | 107368075 | | rs2158836 | | | LAMB1 | Ulcerative colitis-risk | Nat Genet. 2009 |
| | | | | | | | loci on | Feb; 41(2): 216-20. |
| | | | | | | | chromosomes 1p36 | Epub 2009 Jan 4. |
| | | | | | | | and 12g15 found by | |
| | | | | | | | genome-wide | |
| | | | | | | | association study. | |
| chr.8 | none | | | | | | | |
| chr.9 | 138389159 | | | | rs10781500 | — | Genome-wide | Nat Genet. 2009 |
| | | | | | | | association study of | Dec; 41(12): 1330-4. |
| | | | | | | | ulcerative colitis | Epub 2009 Nov |
| | | | | | | | identifies three new | 15. |
| | | | | | | | susceptibility loci, | |
| | | | | | | | including the HNF4A | |
| | | | | | | | region. | |
| chr.10 | none | | | | | | | |
| chr.11 | 116124283 | | | rs28927680 | | BUD13 | Six new loci | Nat Genet. 2008 |
| | | | | | | | associated with | Feb; 40(2): 189-97. |
| | | | | | | | blood low-density | Epub 2008 Jan 13. |
| | | | | | | | lipoprotein | |
| | | | | | | | cholesterol, high- | |
| | | | | | | | density lipoprotein | |
| | | | | | | | cholesterol or | |
| | | | | | | | triglycerides in | |
| | | | | | | | humans. | |
| | 116154127 | | | | rs964184 | — | Common variants at | Nat Genet. 2009 |
| | | | | | | | 30 loci contribute to | Jan; 41(1): 56-65. |
| | | | | | | | polygenic | Epub 2008 Dec 7. |
| | | | | | | | dyslipidemia. | |
| chr.12 | 55351980 | | rs2958154 | | | PTGES3 | Genetic variants | Proc Natl Acad Sci |
| | | | | | | | near TIMP3 and | USA. 2010 Apr |
| | | | | | | | high-density | 20; 107(16): 7401-6. |
| | | | | | | | lipoprotein- | Epub 2010 Apr 12. |
| | | | | | | | associated loci | |
| | | | | | | | influence | |
| | | | | | | | susceptibility to age- | |
| | | | | | | | related macular | |
| | | | | | | | degeneration. | |
| | 64644614 | | | rs1042725 | | HMGA2 | Genome-wide | Nat Genet. 2008 |
| | | | | | | | association analysis | May; 40(5): 575-83. |
| | | | | | | | identifies 20 loci that | Epub 2008 Apr 6. |
| | | | | | | | influence adult | |
| | | | | | | | height. | |
| | 119919970 | | rs2259816 | | | HNF1A | New susceptibility | Nat Genet. 2009 |
| | | | | | | | locus for coronary | Mar: 41(3): 280-2. |
| | | | | | | | artery disease on | Epub 2009 Feb 8. |
| | | | | | | | chromosome | |
| | | | | | | | 3q22.3. | |
| | 119923816 | | | rs1169310 | | HNF1A | Polymorphisms of | Am J Hum Genet. |
| | | | | | | | the HNF1A gene | 2008 |
| | | | | | | | encoding hepatocyte | May: 82(5): 1193-201. |
| | | | | | | | nuclear factor-1 | Epub 2008 |
| | | | | | | | alpha are associated | Apr 24. |
| | | | | | | | with C-reactive | |
| | | | | | | | protein. | |
| chr.13 | 22801791 | | | rs4770433 | | SACS | A genome-wide | PLoS Genet. 2008 |
| | | | | | | | association study | May |
| | | | | | | | identifies protein | 9; 4(5): e1000072. |
| | | | | | | | quantitative trait loci | |
| | | | | | | | (pQTLs). | |
| chr.14 | 102447074 | | | | rs10133111 | — | A genome-wide | Schizophr Bull. |
| | | | | | | | association study of | 2009 Jan: 35(1): 96-108. |
| | | | | | | | schizophrenia using | Epub 2008 |
| | | | | | | | brain activation as a | Nov 20. |
| | | | | | | | quantitative | |
| | | | | | | | phenotype. | |
| chr.15 | none | | | | | | | |
| chr.16 | none | | | | | | | |
| chr.17 | 41074926 | | | | rs393152 | C17orf69 | Genome-wide | Nat Genet. 2009 |
| | | | | | | | association study | Dec: 41(12): 1308-12. |
| | | | | | | | reveals genetic risk | Epub 2009 Nov |
| | | | | | | | underlying | 15. |
| | | | | | | | Parkinson′s disease. | |
| | 44024429 | | | rs9299 | | HOXB5 | A genome-wide | Nat Genet. |
| | | | | | | | association meta- | 2012; 44(5): 526-31. |
| | | | | | | | analysis identifies | |
| | | | | | | | new childhood | |
| | | | | | | | obesity loci. | |
| chr.18 | none | | | | | | | |
| chr.19 | 46002411 | | rs3733829 | | | EGLN2 | Genome-wide meta- | Nat Genet. 2010 |
| | | | | | | | analyses identify | May; 42(5): 441-7. |
| | | | | | | | multiple loci | Epub 2010 Apr 25. |
| | | | | | | | associated with | |
| | | | | | | | smoking behavior. | |
| | 50073874 | | rs6859 | | | PVRL2 | A genome-wide | BMC Med |
| | | | | | | | association study for | Genomics. 2008 |
| | | | | | | | late-onset | Sep 29; 1: 44. |
| | | | | | | | Alzheimer′s disease | |
| | | | | | | | using DNA pooling. | |
| | 62451830 | rs2014572 | | | | ZNF805 | Genome-wide | Am J Med Genet B |
| | | | | | | | association scan of | Neuropsychiatr |
| | | | | | | | quantitative traits for | Genet. 2008 Dec |
| | | | | | | | attention deficit | 5; 147B(8): 1345-54. |
| | | | | | | | hyperactivity | |
| | | | | | | | disorder identifies | |
| | | | | | | | novel associations | |
| | | | | | | | and confirms | |
| | | | | | | | candidate gene | |
| | | | | | | | associations. | |
| | 54915078 | | | | rs3810265 | — | Genome-wide | J Hum Genet. |
| | | | | | | | association study of | 2009 |
| | | | | | | | panic disorder in the | Feb; 54(2): 122-6. |
| | | | | | | | Japanese | Epub 2009 Jan 23. |
| | | | | | | | population. | |
| | 63462695 | | rs260461 | | | ZNF544 | Genome-wide | Am J Med Genet B |
| | | | | | | | association scan of | Neuropsychiatr |
| | | | | | | | quantitative traits for | Genet. 2008 Dec |
| | | | | | | | attention deficit | 5; 147B(8): 1345-54. |
| | | | | | | | hyperactivity | |
| | | | | | | | disorder identifies | |
| | | | | | | | novel associations | |
| | | | | | | | and confirms | |
| | | | | | | | candidate gene | |
| | | | | | | | associations. | |
| chr.20 | 30740755 | | | | rs210135 | — | A genome-wide | Nat Genet. 2009 |
| | | | | | | | meta-analysis | Nov; 41(11): 1182-90. |
| | | | | | | | identifies 22 loci | Epub 2009 Oct |
| | | | | | | | associated with | 11. |
| | | | | | | | eight hematological | |
| | | | | | | | parameters in the | |
| | | | | | | | HaemGen | |
| | | | | | | | consortium. | |
| chr.21 | 43351566 | | rs6586282 | | | CBS | Novel associations | Circ Cardiovasc |
| | | | | | | | of CPS1, MUT, | Genet. 2009 |
| | | | | | | | NOX4, and DPEP1 | Apr; 2(2): 142-50. |
| | | | | | | | with plasma | |
| | | | | | | | homocysteine in a | |
| | | | | | | | healthy population: a | |
| | | | | | | | genome-wide | |
| | | | | | | | evaluation of 13 974 | |
| | | | | | | | participants in the | |
| | | | | | | | Women′s Genome | |
| | | | | | | | Health Study. | |
| chr.22 | 49364219 | | rs5770917 | | | CPT1B | Variant between | Nat Genet. 2008 |
| | | | | | | | CPT1B and CHKB | Nov; 40(11): 1324-8. |
| | | | | | | | associated with | Epub 2008 Sep |
| | | | | | | | susceptibility to | 28. |
| | | | | | | | narcolepsy. | |
| | 49318618 | | | | rs131794 | — | Multiple loci | Nat Genet. 2009 |
| | | | | | | | influence erythrocyte | Nov: 41(11): 1191-8. |
| | | | | | | | phenotypes in the | Epub 2009 Oct |
| | | | | | | | CHARGE | 11. |
| | | | | | | | Consortium. | |
| chr.X | none | | | | | | | |

### REFERENCES

Adam, S. A., Nakagawa, T., Swanson, M. S., Woodruff, T. K., and Dreyfuss, G. (1986). mRNA polyadenylate-binding protein: gene isolation and sequencing and identification of a ribonucleoprotein consensus sequence. Mol Cell Biol 6, 2932-2943.
Andersen, J. S., Lam, Y. W., Leung, A. K., Ong, S. E., Lyon, C. E., Lamond, A. I., and Mann, M. (2005). Nucleolar proteome dynamics. Nature 433, 77-83.
Aravind, L., Iyer, L. M., and Anantharaman, V. (2003). The two faces of Alba: the evolutionary connection between proteins participating in chromatin structure and RNA metabolism. Genome Biol 4, R64.
Ascano, M., Hafner, M., Cekan, P., Gerstberger, S., and Tuschl, T. (2011). Identification of RNA-protein interaction networks using PAR-CLIP. Wiley Interdiscip Rev RNA.
Bessonov, S., Anokhina, M., Will, C. L., Urlaub, H., and Luhrmann, R. (2008). Isolation of an active step I spliceosome and composition of its RNP core. Nature 452, 846-850.
Choi, Y. D., and Dreyfuss, G. (1984). Isolation of the heterogeneous nuclear RNA-ribonucleoprotein complex (hnRNP): a unique supramolecular assembly. Proc Natl Acad Sci USA 81, 7471-7475.
Denhez, F., and Lafyatis, R. (1994). Conservation of regulated alternative splicing and identification of functional domains in vertebrate homologs to the Drosophila splicing regulator, suppressor-of-white-apricot. J Biol Chem 269, 16170-16179.
Dolken, L., Ruzsics, Z., Radle, B., Friedel, C. C., Zimmer, R., Mages, J., Hoffmann, R., Dickinson, P., Forster, T., Ghazal, P., et al. (2008). High-resolution gene expression profiling for simultaneous kinetic parameter analysis of RNA synthesis and decay. RNA 14, 1959-1972.
Drew, K., Winters, P., Butterfoss, G. L., Berstis, V., Uplinger, K., Armstrong, J., Riffle, M., Schweighofer, E., Bovermann, B., Goodlett, D. R., et al. (2011). The Proteome Folding Project: proteome-scale prediction of structure and function. Genome Res 21, 1981-1994.
Favier, D., and Gonda, T. J. (1994). Detection of proteins that bind to the leucine zipper motif of c-Myb. Oncogene 9, 305-311.
Greenberg, J. R. (1979). Ultraviolet light-induced crosslinking of mRNA to proteins. Nucleic Acids Res 6, 715-732.
Haas, S., Steplewski, A., Siracusa, L.D., Amini, S., and Khalili, K. (1995). Identification of a sequence-specific single-stranded DNA binding protein that suppresses transcription of the mouse myelin basic protein gene. J Biol Chem 270, 12503-12510.
Hafner, M., Landgraf, P., Ludwig, J., Rice, A., Ojo, T., Lin, C., Holoch, D., Lim, C., and Tuschl, T. (2008). Identification of microRNAs and other small regulatory RNAs using cDNA library sequencing. Methods 44, 3-12.
Hafner, M., Landthaler, M., Burger, L., Khorshid, M., Hausser, J., Berninger, P., Rothballer, A., Ascano, M., Jr., Jungkamp, A. C., Munschauer, M., et al. (2010). Transcriptome-wide identification of RNA-binding protein and microRNA target sites by PAR-CLIP. Cell 141, 129-141.
Hassfeld, W., Chan, E. K., Mathison, D. A., Portman, D., Dreyfuss, G., Steiner, G., and Tan, E. M. (1998). Molecular definition of heterogeneous nuclear ribonucleoprotein R (hnRNP R) using autoimmune antibody: immunological relationship with hnRNP P. Nucleic Acids Res 26, 439-445.
Hindorff, L. A., Sethupathy, P., Junkins, H. A., Ramos, E. M., Mehta, J. P., Collins, F. S., and Manolio, T. A. (2009). Potential etiologic and functional implications of genome-wide association loci for human diseases and traits. Proc Natl Acad Sci USA 106, 9362-9367.
Jackson, R. J., Hellen, C. U., and Pestova, T. V. (2010). The mechanism of eukaryotic translation initiation and principles of its regulation. Nat Rev Mol Cell Biol 11, 113-127.
Ji Yu, E., Kim, S. H., Heo, K., Ou, C. Y., Stallcup, M. R., and Kim, J. H. (2011). Reciprocal roles of DBC1 and SIRT1 in regulating estrogen receptor {alpha} activity and co-activator synergy. Nucleic Acids Res 39, 6932-6943.
Kabe, Y., Goto, M., Shima, D., Imai, T., Wada, T., Morohashi, K., Shirakawa, M., Hirose, S., and Handa, H. (1999). The role of human MBF1 as a transcriptional coactivator. J Biol Chem 274, 34196-34202.
Kathiresan, S., Melander, O., Guiducci, C., Surti, A., Burtt, N. P., Rieder, M. J., Cooper, G. M., Roos, C., Voight, B. F., Havulinna, A. S., et al. (2008). Six new loci associated with blood low-density lipoprotein cholesterol, high-density lipoprotein cholesterol or triglycerides in humans. Nat Genet. 40, 189-197.
Kathiresan, S., Willer, C. J., Peloso, G. M., Demissie, S., Musunuru, K., Schadt, E. E., Kaplan, L., Bennett, D., Li, Y., Tanaka, T., et al. (2009). Common variants at 30 loci contribute to polygenic dyslipidemia. Nat Genet. 41, 56-65.
Keene, J. D. (2007). RNA regulons: coordination of post-transcriptional events. Nat Rev Genet. 8, 533-543.
Kennedy, M. C., Mende-Mueller, L., Blondin, G. A., and Beinert, H. (1992). Purification and characterization of cytosolic aconitase from beef liver and its relationship to the iron-responsive element binding protein. Proc Natl Acad Sci USA 89, 11730-11734.
Kiledjian, M., and Dreyfuss, G. (1992). Primary structure and binding activity of the hnRNP U protein: binding RNA through RGG box. EMBO J. 11, 2655-2664.
Kim, J. E., Chen, J., and Lou, Z. (2008). DBC1 is a negative regulator of SIRT1. Nature 451, 583-586.
Kishore, S., Jaskiewicz, L., Burger, L., Hausser, J., Khorshid, M., and Zavolan, M. (2011). A quantitative analysis of CLIP methods for identifying binding sites of RNA-binding proteins. Nat Methods 8, 559-564.
Knapinska, A. M., Gratacos, F. M., Krause, C.D., Hernandez, K., Jensen, A. G., Bradley, J. J., Wu, X., Pestka, S., and Brewer, G. (2011). Chaperone Hsp27 modulates AUF1 proteolysis and AU-rich element-mediated mRNA degradation. Mol Cell Biol 31, 1419-1431.
Konig, J., Zarnack, K., Rot, G., Curk, T., Kayikci, M., Zupan, B., Turner, D. J., Luscombe, N. M., and Ule, J. (2010). iCLIP reveals the function of hnRNP particles in splicing at individual nucleotide resolution. Nat Struct Mol Biol 17, 909-915.
Le Hir, H., and Andersen, G. R. (2008). Structural insights into the exon junction complex. Curr Opin Struct Biol 18, 112-119.
Lebedeva, S., Jens, M., Theil, K., Schwanhausser, B., Selbach, M., Landthaler, M., and Rajewsky, N. (2011). Transcriptome-wide Analysis of Regulatory Interactions of the RNA-Binding Protein HuR. Mol Cell 43, 340-352.
Lee, I., and Hong, W. (2004). RAP—a putative RNA-binding domain. Trends Biochem Sci 29, 567-570.
Lindberg, U., and Sundquist, B. (1974). Isolation of messenger ribonucleoproteins from mammalian cells. J Mol Biol 86, 451-468.
Lindblad-Toh, K., Garber, M., Zuk, O., Lin, M. F., Parker, B. J., Washietl, S., Kheradpour, P., Ernst, J., Jordan, G., Mauceli, E., et al. (2011). A high-resolution map of human evolutionary constraint using 29 mammals. Nature 478, 476-482.
Martin, K. C., and Ephrussi, A. (2009). mRNA localization: gene expression in the spatial dimension. Cell 136, 719-730.
Mazan-Mamczarz, K., Galban, S., Lopez de Silanes, I., Martindale, J. L., Atasoy, U., Keene, J. D., and Gorospe, M. (2003). RNA-binding protein HuR enhances p53 translation in response to ultraviolet light irradiation. Proc Natl Acad Sci USA 100, 8354-8359.
Milek, M., Wyler, E., and Landthaler, M. (2011). Transcriptome-wide analysis of protein-RNA interactions using high-throughput sequencing. Semin Cell Dev Biol.
Moore, M. J., and Proudfoot, N. J. (2009). Pre-mRNA processing reaches back to transcription and ahead to translation. Cell 136, 688-700.
Mostafavi, S., Ray, D., Warde-Farley, D., Grouios, C., and Morris, Q. (2008). GeneMANIA: a real-time multiple association network integration algorithm for predicting gene function. Genome Biol 9 Suppl 1, S4.
Nagaraj, N., Wisniewski, J. R., Geiger, T., Cox, J., Kircher, M., Kelso, J., Paabo, S., and Mann, M. (2011). Deep proteome and transcriptome mapping of a human cancer cell line. Mol Syst Biol 7, 548.
Nilsen, T. W., and Graveley, B. R. (2010). Expansion of the eukaryotic proteome by alternative splicing. Nature 463, 457-463.
Ong, S. E., Blagoev, B., Kratchmarova, I., Kristensen, D. B., Steen, H., Pandey, A., and Mann, M. (2002). Stable isotope labeling by amino acids in cell culture, SILAC, as a simple and accurate approach to expression proteomics. Mol Cell Proteomics 1, 376-386.
Owen, H. R., Elser, M., Cheung, E., Gersbach, M., Kraus, W. L., and Hottiger, M. O. (2007). MYBBP1a is a novel repressor of NF-kappaB. J Mol Biol 366, 725-736.
Pena-Castillo, L., Tasan, M., Myers, C. L., Lee, H., Joshi, T., Zhang, C., Guan, Y., Leone, M., Pagnani, A., Kim, W. K., et al. (2008). A critical assessment of Mus musculus gene function prediction using integrated genomic evidence. Genome Biol 9 Suppl 1, S2.
Pollard, K. S., Hubisz, M. J., Rosenbloom, K. R., and Siepel, A. (2010). Detection of normeutral substitution rates on mammalian phylogenies. Genome Res 20, 110-121.
Popow, J., Englert, M., Weitzer, S., Schleiffer, A., Mierzwa, B., Mechtler, K., Trowitzsch, S., Will, C. L., Luhrmann, R., Soil, D., et al. (2011). HSPC117 is the essential subunit of a human tRNA splicing ligase complex. Science 331, 760-764.
Quenault, T., Lithgow, T., and Traven, A. (2011). PUF proteins: repression, activation and mRNA localization. Trends Cell Biol 21, 104-112.
Scherrer, T., Mittal, N., Janga, S.C., and Gerber, A. P. (2010). A screen for RNA-binding proteins in yeast indicates dual functions for many enzymes. PLoS One 5, e15499.
Schmidt, F., Marnef, A., Cheung, M-K., Wilson, I., Hancock, J., Staiger, D. and Ladomery, M. (2010). A protemoic analysis of oligo(dT)-bound mRNP containing oxidative stress-induced Arabidopsis thaliana RNA-binding proteins ATGRP7 and ATGRP8. Mol. Biol. Rep, 37:839-845.
Schwanhausser, B., Busse, D., L1, N., Dittmar, G., Schuchhardt, J., Wolf, J., Chen, W., and Selbach, M. (2011). Global quantification of mammalian gene expression control. Nature 473, 337-342.
Setyono, B., and Greenberg, J. R. (1981). Proteins associated with poly(A) and other regions of mRNA and hnRNA molecules as investigated by crosslinking. Cell 24, 775-783.
Shiina, N., Shinkura, K., and Tokunaga, M. (2005). A novel RNA-binding protein in neuronal RNA granules: regulatory machinery for local translation. J Neurosci 25, 4420-4434.
Silvera, D., Koloteva-Levine, N., Burma, S., and Elroy-Stein, O. (2006). Effect of Ku proteins on IRES-mediated translation. Biol Cell 98, 353-361.
Squires, J. E., Patel, H.R., Nousch, M., Sibbritt, T., Humphreys, D. T., Parker, B. J., Suter, C. M., and Preiss, T. (2012). Widespread occurrence of 5-methylcytosine in human coding and non-coding RNA. Nucleic Acids Res.
Thalhammer, A., Bencokova, Z., Poole, R., Loenarz, C., Adam, J., O'Flaherty, L., Schodel, J., Mole, D., Giaslakiotis, K., Schofield, C. J., et al. (2011). Human AlkB homologue 5 is a nuclear 2-oxoglutarate dependent oxygenase and a direct target of hypoxia-inducible factor 1alpha (HIF-1alpha). PLoS One 6, e16210.
Ting, N. S., Yu, Y., Pohorelic, B., Lees-Miller, S. P., and Beattie, T. L. (2005). Human Ku70/80 interacts directly with hTR, the RNA component of human telomerase. Nucleic Acids Res 33, 2090-2098.
Tsvetanova, N. G., Klass, D. M., Salzman, J., and Brown, P.O. (2010). Proteome-wide search reveals unexpected RNA-binding proteins in Saccharomyces cerevisiae. PLoS One 5.
Ule, J., Jensen, K. B., Ruggiu, M., Mele, A., Ule, A., and Darnell, R. B. (2003). CLIP identifies Nova-regulated RNA networks in the brain. Science 302, 1212-1215.
Vitour, D., Lindenbaum, P., Vende, P., Becker, M. M., and Poncet, D. (2004). RoXaN, a novel cellular protein containing TPR, LD, and zinc finger motifs, forms a ternary complex with eukaryotic initiation factor 4G and rotavirus NSP3. J Virol 78, 3851-3862.
Vogel, C., Abreu Rde, S., Ko, D., Le, S. Y., Shapiro, B. A., Burns, S.C., Sandhu, D., Boutz, D. R., Marcotte, E. M., and Penalva, L. O. (2010). Sequence signatures and mRNA concentration can explain two-thirds of protein abundance variation in a human cell line. Mol Syst Biol 6, 400.
Wagenmakers, A. J., Reinders, R. J., and van Venrooij, W. J. (1980). Cross-linking of mRNA to proteins by irradiation of intact cells with ultraviolet light. Eur J Biochem 112, 323-330.
Wang, E. T., Sandberg, R., Luo, S., Khrebtukova, I., Zhang, L., Mayr, C., Kingsmore, S.F., Schroth, G. P., and Burge, C.B. (2008). Alternative isoform regulation in human tissue transcriptomes. Nature 456, 470-476.
Yoshida, H., Matsui, T., Yamamoto, A., Okada, T., and Mori, K. (2001). XBP1 mRNA is induced by ATF6 and spliced by IRE1 in response to ER stress to produce a highly active transcription factor. Cell 107, 881-891.
Zhang, J., Cho, S. J., Shu, L., Yan, W., Guerrero, T., Kent, M., Skorupski, K., Chen, H., and Chen, X. (2011). Translational repression of p53 by RNPC1, a p53 target overexpressed in lymphomas. Genes Dev 25, 1528-1543.
Zou, T., Mazan-Mamczarz, K., Rao, J. N., Liu, L., Marasa, B. S., Zhang, A.H., Xiao, L., Pullmann, R., Gorospe, M., and Wang, J. Y. (2006). Polyamine depletion increases cytoplasmic levels of RNA-binding protein HuR leading to stabilization of nucleophosmin and p53 mRNAs. J Biol Chem 281, 19387-19394.
Avila-Campillo, I., Drew, K., Lin, J., Reiss, D. J., and Bonneau, R. (2007). BioNetBuilder: automatic integration of biological networks. Bioinformatics 23, 392-393.
Brenner, S. E., Koehl, P., and Levitt, M. (2000). The ASTRAL compendium for protein structure and sequence analysis. Nucleic Acids Res 28, 254-256.
Cline, M. S., Smoot, M., Cerami, E., Kuchinsky, A., Landys, N., Workman, C., Christmas, R., Avila-Campilo, I., Creech, M., Gross, B., et al. (2007). Integration of biological networks and gene expression data using Cytoscape. Nat. Protoc 2, 2366-2382.
Cox, J., and Mann, M. (2008). MaxQuant enables high peptide identification rates, individualized p.p.b.-range mass accuracies and proteome-wide protein quantification. Nat Biotechnol 26, 1367-1372.
Dolken, L., Ruzsics, Z., Radle, B., Friedel, C.C., Zimmer, R., Mages, J., Hoffmann, R., Dickinson, P., Forster, T., Ghazal, P., et al. (2008). High-resolution gene expression profiling for simultaneous kinetic parameter analysis of RNA synthesis and decay. RNA 14, 1959-1972.
Drew, K., Winters, P., Butterfoss, G.L., Berstis, V., Uplinger, K., Armstrong, J., Riffle, M., Schweighofer, E., Bovermann, B., Goodlett, D. R., et al. (2011). The Proteome Folding Project: proteome-scale prediction of structure and function. Genome Res 21, 1981-1994.
Elias, J. E., and Gygi, S. P. (2007). Target-decoy search strategy for increased confidence in large-scale protein identifications by mass spectrometry. Nat Methods 4, 207-214.
Hafner, M., Landgraf, P., Ludwig, J., Rice, A., Ojo, T., Lin, C., Holoch, D., Lim, C., and Tuschl, T. (2008). Identification of microRNAs and other small regulatory RNAs using cDNA library sequencing. Methods 44, 3-12.
Hafner, M., Landthaler, M., Burger, L., Khorshid, M., Hausser, J., Berninger, P., Rothballer, A., Ascano, M., Jr., Jungkamp, A. C., Munschauer, M., et al. (2010). Transcriptome-wide identification of RNA-binding protein and microRNA target sites by PAR-CLIP. Cell 141, 129-141.
Hunter, S., Jones, P., Mitchell, A., Apweiler, R., Attwood, T. K., Bateman, A., Bernard, T., Binns, D., Bork, P., Burge, S., et al. (2011). InterPro in 2011: new developments in the family and domain prediction database. Nucleic Acids Res 40, D306-D312.
Ishihama, Y., Rappsilber, J., Andersen, J. S., and Mann, M. (2002). Microcolumns with self-assembled particle frits for proteomics. Journal of chromatography 979, 233-239.
Konieczka, J. H., Drew, K., Pine, A., Belasco, K., Davey, S., Yatskievych, T. A., Bonneau, R., and Antin, P. B. (2009). BioNetBuilder2.0: bringing systems biology to chicken and other model organisms. BMC Genomics 10 Suppl 2, S6.
Li, H., Handsaker, B., Wysoker, A., Fennell, T., Ruan, J., Homer, N., Marth, G., Abecasis, G., and Durbin, R. (2009). The Sequence Alignment/Map format and SAMtools. Bioinformatics 25, 2078-2079.
McCarthy, F. M., Wang, N., Magee, G. B., Nanduri, B., Lawrence, M. L., Cannon, E. B., Barrell, D. G., Hill, D. P., Dolan, M. E., Williams, W. F., et al. (2006). AgBase: a functional genomics resource for agriculture. BMC Genomics 7, 229.
Mostafavi, S., Ray, D., Warde-Farley, D., Grouios, C., and Morris, Q. (2008). GeneMANIA: a real-time multiple association network integration algorithm for predicting gene function. Genome Biol 9 Suppl 1, S4.
Ong, S. E., Blagoev, B., Kratchmarova, I., Kristensen, D. B., Steen, H., Pandey, A., and Mann, M. (2002). Stable isotope labeling by amino acids in cell culture, SILAC, as a simple and accurate approach to expression proteomics. Mol Cell Proteomics 1, 376-386.
Pena-Castillo, L., Tasan, M., Myers, C. L., Lee, H., Joshi, T., Zhang, C., Guan, Y., Leone, M., Pagnani, A., Kim, W. K., et al. (2008). A critical assessment of Mus musculus gene function prediction using integrated genomic evidence. Genome Biol 9 Suppl 1, S2.
Quinlan, A. R., and Hall, I. M. (2010). BEDTools: a flexible suite of utilities for comparing genomic features. Bioinformatics 26, 841-842.
Rappsilber, J., Mann, M., and Ishihama, Y. (2007). Protocol for micro-purification, enrichment, pre-fractionation and storage of peptides for proteomics using StageTips. Nat Protoc 2, 1896-1906.
Schwanhausser, B., Busse, D., L1, N., Dittmar, G., Schuchhardt, J., Wolf, J., Chen, W., and Selbach, M. (2011). Global quantification of mammalian gene expression control. Nature 473, 337-342.
Shannon, P. T., Reiss, D. J., Bonneau, R., and Baliga, N. S. (2006). The Gaggle: an open-source software system for integrating bioinformatics software and data sources. BMC Bioinformatics 7, 176.
Shevchenko, A., Tomas, H., Havlis, J., Olsen, J. V., and Mann, M. (2006). In-gel digestion for mass spectrometric characterization of proteins and proteomes. Nat Protoc 1, 2856-2860.
Trapnell, C., Pachter, L., and Salzberg, S. L. (2009). TopHat: discovering splice junctions with RNA-Seq. Bioinformatics 25, 1105-1111.
Wozniak, M., Tiuryn, J., and Dutkowski, J. (2010). MODEVO: exploring modularity and evolution of protein interaction networks. Bioinformatics 26, 1790-1791.

1. In vitro method for identifying the sequence of one or more poly(A)+RNA molecules that physically interacts with protein, comprising:
a) formation of poly(A)+RNA-protein complexes via cross-linking,
b) isolation of poly(A)+RNA-protein complexes by
binding of poly(A)+RNA-protein complexes with poly(A)+RNA-binding oligonucleotides, preferably oligo(dT) oligonucleotides, and
removal of unbound poly(A)+RNA, followed by
c) removal of total protein, and
d) identification of poly(A)+RNA sequences.

2. Method according to claim 1, whereby
the cross-linking is carried out by UV irradiation of cells treated with photoreactive nucleosides.

3. Method according to the preceding claim, whereby the photoreactive nucleosides are 4-thiouridine and/or 6-thioguanosine.

4. Method according to the preceding claim, whereby
the cross-linking is carried out by
a) introducing a photoreactive nucleoside into living cells wherein the living cells incorporate the photoreactive nucleoside into RNA transcripts during transcription thereby producing modified RNA transcripts and
b) irradiating said cells at a wavelength significantly absorbed by the photoreactive nucleoside to covalently cross-link a binding site on the modified RNA transcripts to one or more binding proteins, whereby
c) the wavelength is preferably greater than 300 nm.

5. Method according to the preceding claim, whereby the wavelength in step c) is 300-380 nm, preferably between 350-380, more preferably 365 nm.

6. Method according to any one of the preceding claims, whereby
the isolation of poly(A)+RNA-protein complexes is carried out using oligo(dT) oligonucleotides attached to a solid support material.

7. Method according to the preceding claim, whereby the isolation is carried out by
a) forming a soluble extract of the cells,
b) addition of poly(A)+RNA-binding oligonucleotides, preferably oligo(dT) oligonucleotides, attached to a solid support material to said extract,
c) washing the RNA-protein complexes that are bound to said poly(A)+RNA-binding oligonucleotides, preferably oligo(dT) oligonucleotides, attached to a solid support material under denaturing conditions, and
d) treating the extract with a nuclease thereby removing unbound poly(A)+RNA.

8. Method according to any one of the preceding claims, whereby unbound poly(A)+RNA is removed via
a) treatment with one or more RNA-hydrolyzing enzymes, such as RNAse, and/or benzonase,
b) precipitation of protein-poly(A)+RNA complexes, preferably by ammonium sulphate precipitation and/or other protein precipitation methods such as Et-OH, and/or
c) separation according to size, such as by gel electrophoresis, preferably by SDS-PAGE and subsequent transfer of protein-RNA complexes to nitrocellulose.

9. Method according to the preceding claim, whereby unbound poly(A)+RNA is removed via ammonium sulphate precipitation of protein-poly(A)+RNA complexes and separation of said complexes is carried out according to size by gel electrophoresis, preferably by SDS-PAGE, and subsequent transfer of protein-RNA complexes to nitrocellulose, followed preferably by total protein removal by protease K and/or subsequent nucleic acid isolation.

10. Method according to any one of the preceding claims, whereby
total protein is removed via protease treatment.

11. Method according to the preceding claim, whereby
total protein is removed via protease K treatment.

12. Method according to any one of the preceding claims, whereby
poly(A)+RNA sequences are identified via cloning poly(A)+RNA molecules into cDNA libraries followed by sequencing of said libraries.

13. Method according to the preceding claim, whereby
the identification of a sequence of a poly(A)+RNA molecule that physically interacts with protein is determined by
a) identification of a mutation in the sequence of said poly(A)+RNA molecule by sequencing of the purified protein-bound poly(A)+RNA molecules and comparison of said sequence to a reference sequence,
b) whereby the mutation is preferably defined as replacement of a deoxythymidine of the reference sequence by a deoxycytidine, or replacement of a deoxyguanine of the reference sequence by a deoxyadenine in the cDNA of the protein-crosslinked purified poly(A)+RNA molecule of 4-thiouridine and 6-thioguanine labelled cells, respectively, and
c) the sequence of the binding site extends either side of the mutation for at least 1 nucleotide, preferably from 1 to 20 nucleotides.

14. Method according to any one of the preceding claims, whereby
the protein-interaction site is a protein-coding transcript or non-coding transcript.

15. A kit for identifying a protein-interaction site on poly(A)+RNA transcripts, the kit comprising:
a) a thiouridine and/or thioguanosine analog and/or thiouridine and/or thioguanosine analog-supplemented tissue culture medium,
b) reagents for RNA removal, preferably for RNA degradation or for protein-RNA-complex precipitation,
c) reagents for oligo(dT) affinity purification, and
d) adapters and primers for small RNA cloning.

16. Method according to any one of the preceding claims, whereby the sequence of the poly(A)+RNA molecule identified is used to produce an anti-sense oligonucleotide targeted against said sequence of said poly(A)+RNA molecule and said anti-sense oligonucleotide is provided in a pharmaceutically acceptable form comprising preferably a pharmaceutically acceptable carrier.

17. Anti-sense oligonucleotide targeted against the sequence of a poly(A)+RNA molecule identified using the method of any of the preceding claims for use as a medicament, preferably for the treatment of a medical disorder associated with physical interaction between a protein and said poly(A)+RNA sequence.

18. Anti-sense oligonucleotide according to the preceding claim, whereby the oligonucleotide is targeted against a sequence of a poly(A)+RNA molecule comprising a single nucleotide polymorphism (SNP) provided in Table S7 as a medicament for the treatment of a medical disorder associated with said SNP, such as those disorders disclosed in Table S7.

19. Anti-sense oligonucleotide according to the preceding claim, whereby the oligonucleotide binding to the poly(A)+RNA molecule results in changes in expression of the protein for which the poly(A)+RNA molecule codes, either by ribosome disruption, regulation of translation and/or RNA degradation induced by blockage of the binding site of RNA-interacting proteins using anti-sense oligonucleotides.

20. A method for identification of a drug target comprising the method according to any one of the preceding claims, whereby a protein-bound sequence of poly(A)+RNA molecule identified via the method of the preceding claims represents a drug target for treatment with anti-sense oligonucleotides that bind the protein interaction site on the poly(A)+RNA molecule.

21. Method for optimizing a therapeutic antisense oligonucleotide by using the method according to any one of the preceding claims, whereby the sequence of said oligonucleotide is modified according to the protein-binding characteristics of the poly(A)+RNA target molecule.

22. A method for the identification of one or more biomarkers, preferably for identification of a panel or collection of biomarkers, for any given medical condition comprising the method according to any one of the preceding claims, whereby
a) the method is carried out on samples obtained from healthy subjects and affected subjects suffering from said condition, whereby
b) protein-bound sequences of poly(A)+RNA molecules are identified as biomarkers for the medical condition when the presence, extent and/or quantity of protein-binding at the protein-bound sequence of said poly(A)+RNA molecule is significantly different between the two samples.


SEQUENCE LISTING (31 sequences)

SEQ ID NO: 1 - RNA, 52 nt, Artificial
  cloning adapter
rgrururcrargrargrururcrurarcrargrurcrcrgrarcrgrarurc

SEQ ID NO: 2 - DNA, 28 bp, Artificial
  3′ barcoded adapter
tctaaaatcgtatgccgtcttctgcttg

SEQ ID NO: 3 - DNA, 28 bp, Artificial
  3′ barcoded adapter
tctcccatcgtatgccgtcttctgcttg

SEQ ID NO: 4 - DNA, 28 bp, Artificial
  adapter
tctgggatcgtatgccgtcttctgcttg

SEQ ID NO: 5 - DNA, 28 bp, Artificial
  adapter
tcttttatcgtatgccgtcttctgcttg

SEQ ID NO: 6 - DNA, 28 bp, Artificial
  adapter
tctcacgtcgtatgccgtcttctgcttg

SEQ ID NO: 7 - DNA, 28 bp, Artificial
  adapter
tctccattcgtatgccgtcttctgcttg

SEQ ID NO: 8 - DNA, 28 bp, Artificial
  adapter
tctcgtatcgtatgccgtcttctgcttg

SEQ ID NO: 9 - DNA, 28 bp, Artificial
  adapter
tctctgctcgtatgccgtcttctgcttg

SEQ ID NO: 10 - DNA, 49 bp, Artificial
  perimer
ttcagtcgacatggcggccgccagcggctacacggacctgcgtgagaag

SEQ ID NO: 11 - DNA, 26 bp, Artificial
  primer
ctattgatgccaacagcctttccatc

SEQ ID NO: 12 - DNA, 27 bp, Artificial
  primer
atgtcgctttctaacaagctgacgctg

SEQ ID NO: 13 - DNA, 45 bp, Artificial
  primer
ataagaatgcggccgcctaaatattgctgagagcatccaccccag

SEQ ID NO: 14 - DNA, 17 bp, Artificial
  primer
gtgctcgcttcggcagc

SEQ ID NO: 15 - DNA, 21 bp, Artificial
  primer
tggaacgcttcacgaatttgc

SEQ ID NO: 16 - DNA, 19 bp, Artificial
  primer
agccacatcgctcagacac

SEQ ID NO: 17 - DNA, 19 bp, Artificial
  primer
gcccaatacgaccaaatcc

SEQ ID NO: 18 - DNA, 23 bp, Artificial
  primer
tcaagactatctgaagggaatgg

SEQ ID NO: 19 - DNA, 20 bp, Artificial
  primer
caggggttgtgttgaagacc

SEQ ID NO: 20 - DNA, 21 bp, Artificial
  primer
gctagaggcttgatgaatgga

SEQ ID NO: 21 - DNA, 20 bp, Artificial
  primer
gaagggcggtaaccatcata

SEQ ID NO: 22 - DNA, 21 bp, Artificial
  primer
catcaacagctttgaccagtg

SEQ ID NO: 23 - DNA, 20 bp, Artificial
  primer
gccatcaagctcaggctcta

SEQ ID NO: 24 - DNA, 19 bp, Artificial
  primer
ccgattgcatcacaaccat

SEQ ID NO: 25 - DNA, 20 bp, Artificial
  primer
ttagcccatgtcaggacctc

SEQ ID NO: 26 - DNA, 18 bp, Artificial
  primer
gctgtgcgcctaggacat

SEQ ID NO: 27 - DNA, 27 bp, Artificial
  primer
cccttaatgaatttatagaggaacgta

SEQ ID NO: 28 - DNA, 20 bp, Artificial
  primer
tccaggtgaagcagaaaggt

SEQ ID NO: 29 - DNA, 18 bp, Artificial
  primer
ttcttgctgcccaaggag

SEQ ID NO: 30 - DNA, 19 bp, Artificial
  primer
aaattgtgccctgcgagtt

SEQ ID NO: 31 - DNA, 20 bp, Artificial
  primer
atgggagccaaggtaggact